SEQUENCE LISTING (42 sequences)

SEQ ID NO: 1 - protein, 441 aa, homo sapiens [sequence omitted]

SEQ ID NO: 2 - protein, 535 aa, homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 3 - protein, 462 aa, homo sapiens [sequence omitted]

SEQ ID NO: 4 - protein, 705 aa, homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSMYTASENDRLTPSPTDSPRSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 5 - protein, 686 aa, homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 6 - protein, 410 aa, homo sapiens
MFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSMYTASENDRLTPSPTDSPRSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 7 - protein, 134 aa, homo sapiens
MKAFSPVRSVRKNSLSDHSLGISRSKTPVDDPMSLLYNMNDCYSKLKELVPSIPQNKKVSKMEILQHVIDYILDLQIALDSHPTIVSLHHQRPGQNQASRTPLTTLNTDISILSLQASEFPSELMSNDSKALCG

SEQ ID NO: 8 - protein, 443 aa, homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 9 - protein, 527 aa, homo sapiens
MTAELREAMALAPWGPVKVKKEEEEEENFPGQASSQQVHSENIKVWAPVQGLQTGLDGSEEEEKGQNISWDMAVVLKATQEAPAASTLGSYSLPGTLAKSEILETHGTMNFLGAETKNLQLLVPKTEICEEAEKPLIISERIQKADPQGPELGEACEKGNMLKRQRIKREKKDFRQVIVNDCHLPESFKEEENQKCKKSGGKYSLNSGAVKNPKTQLGQKPFTCSVCGKGFSQSANLVVHQRIHTGEKPFECHECGKAFIQSANLVVHQRIHTGQKPYVCSKCGKAFTQSSNLTVHQKIHSLEKTFKCNECEKAFSYSSQLARHQKVHITEKCYECNECGKTFTRSSNLIVHQRIHTGEKPFACNDCGKAFTQSANLIVHQRSHTGEKPYECKECGKAFSCFSHLIVHQRIHTAEKPYDCSECGKAFSQLSCLIVHQRIHSGDLPYVCNECGKAFTCSSYLLIHQRIHNGEKPYTCNECGKAFRQRSSLTVHQRTHTGEKPYECEKCGAAFISNSHLMRHHRTHLVE

SEQ ID NO: 10 - protein, 509 aa, homo sapiens
MEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 11 - protein, 526 aa, homo sapiens
MDVRFYPPPAQPAAAPDAPCLGPSPCLDPYYCNKFDGENMYMSMTEPSQDYVPASQSYPGPSLESEDFNIPPITPPSLPDHSLVHLNEVESGYHSLCHPMNHNGLLPFHPQNMDLPEITVSNMLGQDGTLLSNSISVMPDIRNPEGTQYSSHPQMAAMRPRGQPADIRQQPGMMPHGQLTTINQSQLSAQLGLNMGGSNVPHNSPSPPGSKSATPSPSSSVHEDEGDDTSKINGGEKRPASDMGKKPKTPKKKKKKDPNEPQKPVSAYALFFRDTQAAIKGQNPNATFGEVSKIVASMWDGLGEEQKQVYKKKTEAAKKEYLKQLAAYRASLVSKSYSEPVDVKTSQPPQLINSKPSVFHGPSQAHSALYLSSHYHQQPGMNPHLTAMHPSLPRNIAPKPNNQMPVTVSIANMAVSPPPPLQISPPLHQHLNMQQHQPLTMQQPLGNQLPMQVQSALHSPTMQQGFTLQPDYQTIINPTSTAAQVVTQAMEYVRSGCRNPPPQPVDWNNDYCSSGGMQRDKALYLT

SEQ ID NO: 12 - protein, 429 aa, homo sapiens
MASNSIFDSFPTYSPTFIRDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 13 - protein, 402 aa, homo sapiens
MNIHMKRKTIKNINTFENRMLMLDGMPAVRVKTELLESEQGSPNVHNYPDMEAVPLLLNNVKGEPPEDSLSVDHFQTQTEPVDLSINKARTSPTAVSSSPVSMTASASSPSSTSTSSSSSSRLASSPTVITSVSSASSSSTVLTPGPLVASASGVGGQQFLHIIHPVPPSSPMNLQSNKLSHVHRIPVVVQSVPVVYTAVRSPGNVNNTIVVPLLEDGRGHGKAQMDPRGLSPRQSKSDSDDDDLPNVTLDSVNETGSTALSIARAVQEVHPSPVSRVRGNRMNNQKFPCSISPFSIESTRRQRRSESPDSRKRRIHRCDFEGCNKVYTKSSHLKAHRRTHTGEKPYKCTWEGCTWKFARSDELTRHYRKHTGVKPFKCADCDRSFSRSDHLALHRRRHMLV

SEQ ID NO: 14 - protein, 1214 aa, homo sapiens
MKQPIMADGPRCKRRKQANPRRKNVVNYDNVVDTGSETDEEDKLHIAEDDGIANPLDQETSPASVPNHESSPHVSQALLPREEEEDEIREGGVEHPWHNNEILQASVDGPEEMKEDYDTMGPEATIQTAINNGTVKNANCTSDFEEYFAKRKLEERDGHAVSIEEYLQRSDTAIIYPEAPEELSRLGTPEANGQEENDLPPGTPDAFAQLLTCPYCDRGYKRLTSLKEHIKYRHEKNEENFSCPLCSYTFAYRTQLERHMVTHKPGTDQHQMLTQGAGNRKFKCTECGKAFKYKHHLKEHLRIHSGEKPYECPNCKKRFSHSGSYSSHISSKKCIGLISVNGRMRNNIKTGSSPNSVSSSPTNSAITQLRNKLENGKPLSMSEQTGLLKIKTEPLDFNDYKVLMATHGFSGTSPFMNGGLGATSPLGVHPSAQSPMQHLGVGMEAPLLGFPTMNSNLSEVQKVLQIVDNTVSRQKMDCKAEEISKLKGYHMKDPCSQPEEQGVTSPNIPPVGLPVVSHNGATKSIIDYTLEKVNEAKACLQSLTTDSRRQISNIKKEKLRTLIDLVTDDKMIENHNISTPFSCQFCKESFPGPIPLHQHERYLCKMNEEIKAVLQPHENIVPNKAGVFVDNKALLLSSVLSEKGMTSPINPYKDHMSVLKAYYAMNMEPNSDELLKISIAVGLPQEFVKEWFEQRKVYQYSNSRSPSLERSSKPLAPNSNPPTKDSLLPRSPVKPMDSITSPSIAELHNSVTNCDPPLRLTKPSHFTNIKPVEKLDHSRSNTPSPLNLSSTSSKNSHSSSYTPNSFSSEELQAEPLDLSLPKQMKEPKSIIATKNKTKASSISLDHNSVSSSSENSDEPLNLTFIKKEFSNSNNLDNKSTNPVFSMNPFSAKPLYTALPPQSAFPPATFMPPVQTSIPGLRPYPGLDQMSFLPHMAYTYPTGAATFADMQQRRKYQRKQGFQGELLDGAQDYMSGLDDMTDSDSCLSRKKIKKTESGMYACDLCDKTFQKSSSLLRHKYEHTGKRPHQCQICKKAFKHKHHLIEHSRLHSGEKPYQCDKCGKRFSHSGSYSQHMNHRYSYCKREAEEREAAEREAREKGHLEPTELLMNRAYLQSITPQGYSDSEERESMPRDGESEKEHEKEGEDGYGKLGRQDGDEEFEEEEEESENKSMDTDPETIRDEEETGDHSMDDSSEDGKMETKSDHEEDNMEDGM

SEQ ID NO: 15 - protein, 349 aa, homo sapiens
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLPLSERPSKKGKKPKTEKEDKVKHIKQEPVESSLGLSNGVSDLSPEYAVLTSTIKNEVDSTVNIIVVGQSHLDSNIENQEIVTNPPDICQVVEVTTESDEQPVSMSELYPLQISPVSSYAESETTDSVPSDEESAEGRPHWRKRNIEGKQYLSNMGTRGSYLLPGMASFVTSNKPDLQVTIKEESNPVPYNSSWPPFQDLPLSSSMTPASSSSRPDRETRASVIKKTSDITQARVKSC

SEQ ID NO: 16 - protein, 793 aa, homo sapiens
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSL

SEQ ID NO: 17 - protein, 519 aa, homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 18 - protein, 663 aa, homo sapiens
MAITLQPSDLIFEFASNGMDDDIHQLEDPSVFPAVIVEQVPYPDLLHLYSGLELDDVHNGIITDGTLCMTQDQILEGSFLLTDDNEATSHTMSTAEVLLNMESPSDILDEKQIFSTSEMLPDSDPAPAVTLPNYLFPASEPDALNRAGDTSDQEGHSLEEKASREESAKKTGKSKKRIRKTKGNRSTSPVTDPSIPIRKKSKDGKGSTIYLWEFLLALLQDRNTCPKYIKWTQREKGIFKLVDSKAVSKLWGKQKNKPDMNYETMGRALRYYYQRGILAKVEGQRLVYQFKEMPKDLVVIEDEDESSEATAAPPQASTASVASASTTRRTSSRVSSRSAPQGKGSSSWEKPKIQHVGLQPSASLELGPSLDEEIPTTSTMLVSPAEGQVKLTKAVSASSVPSNIHLGVAPVGSGSALTLQTIPLTTVLTNGPPASTTAPTQLVLQSVPAASTFKDTFTLQASFPLNASFQDSQVAAPGAPLILSGLPQLLAGANRPTNPAPPTVTGAGPAGPSSQPPGTVIAAFIRTSGTTAAPRVKEGPLRSSSYVQGMVTGAPMEGLLVPEETLRELLRDQAHLQPLPTQVVSRGSHNPSLLGNQTLSPPSRPTVGLTPVAELELSSGSGSLLMAEPSVTTSGSLLTRSPTPAPFSPFNPTSLIKMEPHDI

SEQ ID NO: 19 - protein, 673 aa, homo sapiens
MDLTKMGMIQLQNPSHPTGLLCKANQMRLAGTLCDVVIMVDSQEFHAHRTVLACTSKMFEILFHRNSQHYTLDFLSPKTFQQILEYAYTATLQAKAEDLDDLLYAAEILEIEYLEEQCLKMLETIQASDDNDTEATMADGGAEEEEDRKARYLKNIFISKHSSEESGYASVAGQSLPGPMVDQSPSVSTSFGLSAMSPTKAAVDSLMTIGQSLLQGTLQPPAGPEEPTLAGGGRHPGVAEVKTEMMQVDEVPSQDSPGAAESSISGGMGDKVEERGKEGPGTPTRSSVITSARELHYGREESAEQVPPPAEAGQAPTGRPEHPAPPPEKHLGIYSVLPNHKADAVLSMPSSVTSGLHVQPALAVSMDFSTYGGLLPQGFIQRELFSKLGELAVGMKSESRTIGEQCSVCGVELPDNEAVEQHRKLHSGMKTYGCELCGKRFLDSLRLRMHLLAHSAGAKAFVCDQCGAQFSKEDALETHRQTHTGTDMAVFCLLCGKRFQAQSALQQHMEVHAGVRSYICSECNRTFPSHTALKRHLRSHTGDHPYECEFCGSCFRDESTLKSHKRIHTGEKPYECNGCGKKFSLKHQLETHYRVHTGEKPFECKLCHQRSRDYSAMIKHLRTHNGASPYQCTICTEYCPSLSSMQKHMKGHKPEEIPPDWRIEKTYLYLCYV

SEQ ID NO: 20 - protein, 480 aa, homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 21 - protein, 619 aa, homo sapiens
MAAVVQQNDLVFEFASNVMEDERQLGDPAIFPAVIVEHVPGADILNSYAGLACVEEPNDMITESSLDVAEEEIIDDDDDDITLTVEASCHDGDETIETIEAAEALLNMDSPGPMLDEKRINNNIFSSPEDDMVVAPVTHVSVTLDGIPEVMETQQVQEKYADSPGASSPEQPKRKKGRKTKPPRPDSPATTPNISVKKKNKDGKGNTIYLWEFLLALLQDKATCPKYIKWTQREKGIFKLVDSKAVSRLWGKHKNKPDMNYETMGRALRYYYQRGILAKVEGQRLVYQFKEMPKDLIYINDEDPSSSIESSDPSLSSSATSNRNQTSRSRVSSSPGVKGGATTVLKPGNSKAAKPKDPVEVAQPSEVLRTVQPTQSPYPTQLFRTVHVVQPVQAVPEGEAARTSTMQDETLNSSVQSIRTIQAPTQVPVVVSPRNQQLHTVTLQTVPLTTVIASTDPSAGTGSQKFILQAIPSSQPMTVLKENVMLQSQKAGSPPSIVLGPAQVQQVLTSNVQTICNGTVSVASSPSFSATAPVVTFSPRSSQLVAHPPGTVITSVIKTQETKTLTQEVEKKESEDHLKENTEKTEQQPQPYVMVVSSSNGFTSQVAMKQNELLEPNSF

SEQ ID NO: 22 - DNA, 1325 bp, homo sapiens
atgaaggcggccgtcgatctcaagccgactctcaccatcatcaagacggaaaaagtcgatctggagcttttcccctccccggatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactaccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctacggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagtga

SEQ ID NO: 23 - DNA, 2583 bp, homo sapiens
agtgacagcggcccgctggagaggaagcccgagagctgccgcgcgcctgccggacgagggcgtagaagccaggcgtcagagcccgggctccggtggggtcccccacccggccctcgggtcccccgccccctgctccctgcccatcccagcccacgcgaccctctcgcgcgcggaggggcgggtcctcgacggctacgggaaggtgccagcccgccccggatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactgagcagatgacatgatgaaaggaacagaaacagtgttattaggttggaggacaccgactaatttgggaaacggatgaaggactgagaaggcccccgctccctctggcccttctctgtttagtagttggttggggaagtggggctcaagaaggattttggggttcaccagatgcttcctggcccacgatgaaacctgagaggggtgtccccttgccccatcctctgccctaactacagtcgtttacctggtgctgcgtcttgcttttggtttccagctggagaaaagaagacaagaaagtcttgggcatgaaggagctttttgcatctagtgggtgggaggggtcaggtgtgggacatgggagcaggagactccactttcttcctttgtacagtaactttcaaccttttcgttggcatgtgtgttaatccctgatccaaaaagaacaaatacacgtatgttataaccatcagcccgccagggtcagggaaaggactcacctgactttggacagctggcctgggctccccctgctcaaacacagtggggatcagagaaaaggggctggaaaggggggaatggcccacatctcaagaagcaagatattgtttgtggtggttgtgtgtgggtgtgtgttttttctttttctttctttttattttttttgaatgggggaggctatttattgtactgagagtggtgtctggatatattccttttgtcttcatcactttctgaaaataaacataaaactgttgaa

SEQ ID NO: 24 - DNA, 1427 bp, homo sapiens
atgcagctgagaaaaatgcagaccgtcaaaaaggagcaggcgtctcttgatgccagtagcaatgtggacaagatgatggtccttaattctgctttaacggaagtgtcagaagactccacaacaggtgaggacgtgcttctcagtgaaggaagtgtggggaagaacaaatcttctgcatgtcggaggaaacgggaattcattcctgatgaaaagaaagatgctatgtattgggaaaaaaggcggaaaaataatgaagctgccaaaagatctcgtgagaagcgtcgactgaatgacctggttttagagaacaaactaattgcactgggagaagaaaacgccactttaaaagctgagctgctttcactaaaattaaagtttggtttaattagctccacagcatatgctcaagagattcagaaactcagtaattctacagctgtgtactttcaagattaccagacttccaaatccaatgtgagttcatttgtggacgagcacgaaccctcgatggtgtcaagtagttgtatttctgtcattaaacactctccacaaagctcgctgtccgatgtttcagaagtgtcctcagtagaacacacgcaggagagctctgtgcagggaagctgcagaagtcctgaaaacaagttccagattatcaagcaagagccgatggaattagagagctacacaagggagccaagagatgaccgaggctcttacacagcgtccatctatcaaaactatatggggaattctttctctgggtactcacactctcccccactactgcaagtcaaccgatcctccagcaactccccgagaacgtcggaaactgatgatggtgtggtaggaaagtcatctgatggagaagacgagcaacaggtccccaagggccccatccattctccagttgaactcaagcatgtgcatgcaactgtggttaaagttccagaagtgaattcctctgccttgccacacaagctccggatcaaagccaaagccatgcagatcaaagtagaagcctttgataatgaatttgaggccacgcaaaaactttcctcacctattgacatgacatctaaaagacatttcgaactcgaaaagcatagtgccccaagtatggtacattcttctcttactcctttctcagtgcaagtgactaacattcaagattggtctctcaaatcggagcactggcatcaaaaagaactgagtggcaaaactcagaatagttccaaaactggagttgttgaaatgaaagacagtggctacaaagtttctgacccagagaacttgtatttgaagcaggggatagcaaacttatctgcagaggttgtctcactcaagagacttatagccacacaaccaatctctgcttcagactctgggtaaattactactgagtaagagctgggcatttagaaagatgt

SEQ ID NO: 25 - DNA, 3264 bp, homo sapiens
agccagctgccgtctgcgctttgcaaagcatgcagttaggggagcagctcttggtgagctcagtgaacctgcctggcgcgcacttctacccgctggagagtgcgcgaggcggcagcggcgggagcgctggccacctccccagcgcggccccctctcctcagaagttggacttagacaaagcgtccaagaagttttccggcagtctctcctgcgaggcggtgagcggggagcccgcagccgccagcgcaggggcccccgcggccatgcttagtgacaccgacgccggggacgcatttgccagcgctgcggcagtggccaagccggggcccccggacggccgcaagggctccccctgcggggaggaggagctgccctccgccgctgcagccgccgccgccgccgccgccgcggctgcggccactgcgcgctactccatggacagcctgagctccgagcggtactacctccagtcccccggtcctcaggggtcggagctggctgcgccctgctcactcttcccgtaccaggcggcggctggggcgccccacggacctgtgtacccggctcctaacggggcgcgctacccctacggctccatgctgccccccggcggcttccccgcggctgtgtgcccacccgggagggcgcagttcggcccaggagccggtgcgggcagtggcgcgggcggtagcagcggcgggggcggcggcccgggcacctatcagtacagccagggggctccgctctacgggccgtaccctggagccgcagcggcgggatcttgcggaggactggggggcctgggggttccaggttctggcttccgtgcccacgtctacctgtgcaaccggcctctgtggctcaaattccaccgccaccaaactgagatgatcattacgaaacagggcaggcgcatgtttcctttcttgagcttcaacataaacggactcaatcccactgcccactacaatgtgttcgtagaggtggtgctggcggaccccaaccactggcgcttccaggggggcaaatgggtgacctgtggcaaagccgacaataacatgcagggcaacaaaatgtatgttcacccagagtctcctaatactggttcccactggatgagacaggagatttcattcgggaaattaaaactcaccaataacaaaggcgcaaataacaacaacacccagatgatagtcttacaatccttacacaaataccaaccccgactgcatattgttgaagttacagaggatggcgtggaggacttgaatgagccctcaaagacccagacttttaccttctcagaaacgcaattcattgcagtgactgcctaccaaaacaccgatattactcaactaaagattgatcataacccctttgcaaaaggcttcagagacaactatgattccatgtacaccgcttcagaaaatgacaggttaactccatctcccacggattctcctagatcccatcagattgtccctggaggtcggtacggcgttcaatccttcttcccggagccctttgtcaacactttacctcaagcccgctattataatggcgagagaaccgtgccacagaccaacggcctcctttcaccccaacagagcgaagaggtggccaaccctccccagcggtggcttgtcacgcctgtccagcaacctgggaccaacaaactagacatcagttcctatgaatctgaatatacttctagcacattgctcccatatggcattaaatccttgccccttcagacatcccatgccctggggtattacccagacccaacctttcctgcaatggcagggtggggaggtcgaggttcttaccagaggaagatggcagctggactaccatggacctccagaacaagccccactgtgttctctgaagatcagctctccaaggagaaagtgaaagaggaaattggctcttcttggatagagacacccccttccatcaaatctctagattccaatgattcaggagtatacaccagtgcttgtaagcgaaggcggctgtctcctagcaactccagtaatgaaaattcaccctccataaagtgtgaggacattaatgctgaagagtatagtaaagacacctcaaaaggcatgggagggtattatgctttttacacaactccctaaagagttattttaacctcaaaaattagctaactttttgcagatggacttggtggtgttttttgttgtcttctttgcctaggttgccaaaaagatgtttgccttccaccttgatgcatcctgttttgtgcaattctctaaaagaaggtgccaaagctttttgattgctgcaggtaactgaaacaaacctagcatttttaaaaaataagattaatggaagactttaaggtattttaaaattcgaagggtatccaaggttctgtatttatttattggggagacactaacccttcaaagaagcaggctgtgaacattgggtgcccagtgctatcagatgagttaaaacctttgattctcatttctatttgtaaattcttaagcaaatagaagccgagtgttaaggtgttttgcttctgaaagagggctgtgccttccgtttcagaaggagacattttgctgttacattctgccaggggcaaaagatactaggcccaggagtcaagaaaagcttttgtgaaagtgatagtttcacctgactttgattccttaacccccggcttttggaacaagccatgtttgccctagtccaggattgcctcacttgagacttgctaggcctctgctgtgtgctggggtggccagtgggactcaggagagagcaagctaaggagtcaccaaaaaaaaaaaaaaaaaaaagggagaatttaaaagtgtacagttgtgtgtttagatacactatagaataatgtggtatatattgtacaaatagtctacataggtgtctgggataatgtaaaactggtgctttggctttgtaaagaatttgcaaatcacttaacagctgcaggggcaaggggagagtttcatcatccccatgatatttgggaatattctgtttacttcttagatagttaagaatgtattcagctactatgtactaacttgaaccgtgtttaaggaaaactcctatttcatcctcttcttgcgccatcccctctccctaacttggtaatgtgaagaaactaaaacctgataccacagctcctataggcattttagagatcttggatttttatgtacagtcttagtcatttttaataaatgtggttcagtaagggaa

SEQ ID NO: 26 - DNA, 3207 bp, homo sapiens
agccagctgccgtctgcgctttgcaaagcatgcagttaggggagcagctcttggtgagctcagtgaacctgcctggcgcgcacttctacccgctggagagtgcgcgaggcggcagcggcgggagcgctggccacctccccagcgcggccccctctcctcagaagttggacttagacaaagcgtccaagaagttttccggcagtctctcctgcgaggcggtgagcggggagcccgcagccgccagcgcaggggcccccgcggccatgcttagtgacaccgacgccggggacgcatttgccagcgctgcggcagtggccaagccggggcccccggacggccgcaagggctccccctgcggggaggaggagctgccctccgccgctgcagccgccgccgccgccgccgccgcggctgcggccactgcgcgctactccatggacagcctgagctccgagcggtactacctccagtcccccggtcctcaggggtcggagctggctgcgccctgctcactcttcccgtaccaggcggcggctggggcgccccacggacctgtgtacccggctcctaacggggcgcgctacccctacggctccatgctgccccccggcggcttccccgcggctgtgtgcccacccgggagggcgcagttcggcccaggagccggtgcgggcagtggcgcgggcggtagcagcggcgggggcggcggcccgggcacctatcagtacagccagggggctccgctctacgggccgtaccctggagccgcagcggcgggatcttgcggaggactggggggcctgggggttccaggttctggcttccgtgcccacgtctacctgtgcaaccggcctctgtggctcaaattccaccgccaccaaactgagatgatcattacgaaacagggcaggcgcatgtttcctttcttgagcttcaacataaacggactcaatcccactgcccactacaatgtgttcgtagaggtggtgctggcggaccccaaccactggcgcttccaggggggcaaatgggtgacctgtggcaaagccgacaataacatgcagggcaacaaaatgtatgttcacccagagtctcctaatactggttcccactggatgagacaggagatttcattcgggaaattaaaactcaccaataacaaaggcgcaaataacaacaacacccagatgatagtcttacaatccttacacaaataccaaccccgactgcatattgttgaagttacagaggatggcgtggaggacttgaatgagccctcaaagacccagacttttaccttctcagaaacgcaattcattgcagtgactgcctaccaaaacaccgatattactcaactaaagattgatcataacccctttgcaaaaggcttcagagacaactatgattcatcccatcagattgtccctggaggtcggtacggcgttcaatccttcttcccggagccctttgtcaacactttacctcaagcccgctattataatggcgagagaaccgtgccacagaccaacggcctcctttcaccccaacagagcgaagaggtggccaaccctccccagcggtggcttgtcacgcctgtccagcaacctgggaccaacaaactagacatcagttcctatgaatctgaatatacttctagcacattgctcccatatggcattaaatccttgccccttcagacatcccatgccctggggtattacccagacccaacctttcctgcaatggcagggtggggaggtcgaggttcttaccagaggaagatggcagctggactaccatggacctccagaacaagccccactgtgttctctgaagatcagctctccaaggagaaagtgaaagaggaaattggctcttcttggatagagacacccccttccatcaaatctctagattccaatgattcaggagtatacaccagtgcttgtaagcgaaggcggctgtctcctagcaactccagtaatgaaaattcaccctccataaagtgtgaggacattaatgctgaagagtatagtaaagacacctcaaaaggcatgggagggtattatgctttttacacaactccctaaagagttattttaacctcaaaaattagctaactttttgcagatggacttggtggtgttttttgttgtcttctttgcctaggttgccaaaaagatgtttgccttccaccttgatgcatcctgttttgtgcaattctctaaaagaaggtgccaaagctttttgattgctgcaggtaactgaaacaaacctagcatttttaaaaaataagattaatggaagactttaaggtattttaaaattcgaagggtatccaaggttctgtatttatttattggggagacactaacccttcaaagaagcaggctgtgaacattgggtgcccagtgctatcagatgagttaaaacctttgattctcatttctatttgtaaattcttaagcaaatagaagccgagtgttaaggtgttttgcttctgaaagagggctgtgccttccgtttcagaaggagacattttgctgttacattctgccaggggcaaaagatactaggcccaggagtcaagaaaagcttttgtgaaagtgatagtttcacctgactttgattccttaacccccggcttttggaacaagccatgtttgccctagtccaggattgcctcacttgagacttgctaggcctctgctgtgtgctggggtggccagtgggactcaggagagagcaagctaaggagtcaccaaaaaaaaaaaaaaaaaaaagggagaatttaaaagtgtacagttgtgtgtttagatacactatagaataatgtggtatatattgtacaaatagtctacataggtgtctgggataatgtaaaactggtgctttggctttgtaaagaatttgcaaatcacttaacagctgcaggggcaaggggagagtttcatcatccccatgatatttgggaatattctgtttacttcttagatagttaagaatgtattcagctactatgtactaacttgaaccgtgtttaaggaaaactcctatttcatcctcttcttgcgccatcccctctccctaacttggtaatgtgaagaaactaaaacctgataccacagctcctataggcattttagagatcttggatttttatgtacagtcttagtcatttttaataaatgtggttcagtaagggaa

SEQ ID NO: 27 - DNA, 2638 bp, homo sapiens
gtttccaagtggtcaacttgaccgatgctttggcaattgaaaaagggcagaaaggcgcgggctagtgggtggatggggacaaagatctaagtcaccttcttccagcgtgtgagcctgggaggagggtgggggtcctgaggagcaagaggtacgaggaaggaaaaggagagggcttctgggttagtttccacctcctgctttccaactcacggcgctttccttccggaaaggacgctggattcagggcgcgccagtacgcgcagtagcggcccgcgagtcggcaggcgcatgtttcctttcttgagcttcaacataaacggactcaatcccactgcccactacaatgtgttcgtagaggtggtgctggcggaccccaaccactggcgcttccaggggggcaaatgggtgacctgtggcaaagccgacaataacatgcagggcaacaaaatgtatgttcacccagagtctcctaatactggttcccactggatgagacaggagatttcattcgggaaattaaaactcaccaataacaaaggcgcaaataacaacaacacccagatgatagtcttacaatccttacacaaataccaaccccgactgcatattgttgaagttacagaggatggcgtggaggacttgaatgagccctcaaagacccagacttttaccttctcagaaacgcaattcattgcagtgactgcctaccaaaacaccgatattactcaactaaagattgatcataacccctttgcaaaaggcttcagagacaactatgattccatgtacaccgcttcagaaaatgacaggttaactccatctcccacggattctcctagatcccatcagattgtccctggaggtcggtacggcgttcaatccttcttcccggagccctttgtcaacactttacctcaagcccgctattataatggcgagagaaccgtgccacagaccaacggcctcctttcaccccaacagagcgaagaggtggccaaccctccccagcggtggcttgtcacgcctgtccagcaacctgggaccaacaaactagacatcagttcctatgaatctgaatatacttctagcacattgctcccatatggcattaaatccttgccccttcagacatcccatgccctggggtattacccagacccaacctttcctgcaatggcagggtggggaggtcgaggttcttaccagaggaagatggcagctggactaccatggacctccagaacaagccccactgtgttctctgaagatcagctctccaaggagaaagtgaaagaggaaattggctcttcttggatagagacacccccttccatcaaatctctagattccaatgattcaggagtatacaccagtgcttgtaagcgaaggcggctgtctcctagcaactccagtaatgaaaattcaccctccataaagtgtgaggacattaatgctgaagagtatagtaaagacacctcaaaaggcatgggagggtattatgctttttacacaactccctaaagagttattttaacctcaaaaattagctaactttttgcagatggacttggtggtgttttttgttgtcttctttgcctaggttgccaaaaagatgtttgccttccaccttgatgcatcctgttttgtgcaattctctaaaagaaggtgccaaagctttttgattgctgcaggtaactgaaacaaacctagcatttttaaaaaataagattaatggaagactttaaggtattttaaaattcgaagggtatccaaggttctgtatttatttattggggagacactaacccttcaaagaagcaggctgtgaacattgggtgcccagtgctatcagatgagttaaaacctttgattctcatttctatttgtaaattcttaagcaaatagaagccgagtgttaaggtgttttgcttctgaaagagggctgtgccttccgtttcagaaggagacattttgctgttacattctgccaggggcaaaagatactaggcccaggagtcaagaaaagcttttgtgaaagtgatagtttcacctgactttgattccttaacccccggcttttggaacaagccatgtttgccctagtccaggattgcctcacttgagacttgctaggcctctgctgtgtgctggggtggccagtgggactcaggagagagcaagctaaggagtcaccaaaaaaaaaaaaaaaaaaaagggagaatttaaaagtgtacagttgtgtgtttagatacactatagaataatgtggtatatattgtacaaatagtctacataggtgtctgggataatgtaaaactggtgctttggctttgtaaagaatttgcaaatcacttaacagctgcaggggcaaggggagagtttcatcatccccatgatatttgggaatattctgtttacttcttagatagttaagaatgtattcagctactatgtactaacttgaaccgtgtttaaggaaaactcctatttcatcctcttcttgcgccatcccctctccctaacttggtaatgtgaagaaactaaaacctgataccacagctcctataggcattttagagatcttggatttttatgtacagtcttagtcatttttaataaatgtggttcagtaagggaa

SEQ ID NO: 28 - DNA, 1299 bp, homo sapiens
attctgagccgagcccggtgccaagcgcagctagctcagcaggcggcagcggcggcctgagcttcagggcagccagctccctcccggtctcgccttccctcgcggtcagcatgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctgaataagcggtgttcatgatttcttttattctttgcacaacaacaacaacaacaaattcacggaatcttttaagtgctgaacttatttttcaaccatttcacaaggaggacaagttgaatggacctttttaaaaagaaaaaaaaaatggaaggaaaactaagaatgatcatcttcccagggtgttctcttacttggactgtgatattcgttatttatgaaaaagacttttaaatgccctttctgcagttggaaggttttctttatatactattcccaccatggggagcgaaaacgttaaaatcacaaggaattgcccaatctaagcagactttgccttttttcaaaggtggagcgtgaataccagaaggatccagtattcagtcacttaaatgaagtcttttggtcagaaattacctttttgacacaagcctactgaatgctgtgtatatatttatatataaatatatctatttgagtgaaaccttgtgaactctttaattagagttttcttgtatagtggcagagatgtctatttctgcattcaaaagtgtaatgatgtacttattcatgctaaactttttataaaagtttagttgtaaacttaacccttttatacaaaataaatcaagtgtgtttattgaatggtgattgcctgctttatttcagaggaccagtgctttgatttttattatgctatgttataactgaacccaaataaatacaagttcaaatttatgtagactgtataagattataataaaacatgtctgaagtcaatacctgaa

SEQ ID NO: 29 - DNA, 3083 bp, homo sapiens
gaacactgagctgcctggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaacta

SEQ ID NO: 30 - DNA, 2658 bp, homo sapiens
ctgtccggtcattgttctcgtgccggtagaagctgaagtaccggtcagccagcctcctgctgcagagccagtgaactcaggtcgggcttctcagctgcgcacataggcagtactcatcttggccctgggaagaaactcaagaagaagcttttgaaacataaagcttggatggggtttgacctctgcagggcagcgcccagctataggagttcccctgctgagcagagaagatgactgcagaattgagagaagccatggccctagccccatggggcccagtgaaggtgaaaaaggaggaggaagaagaagaaaacttcccaggtcaggcatccagccaacaagtgcactccgagaacatcaaagtctgggccccagtgcagggtcttcagacaggccttgatggatcagaagaggaagaaaagggtcagaacatatcctgggatatggcggtagtcctgaaagcaactcaggaggcacctgctgcttcaacccttggcagctactcattaccagggactctggccaagagtgagatactggagactcatgggaccatgaactttctaggtgctgaaaccaagaacctacagttactggttccaaaaactgagatatgtgaggaagctgaaaaacccctcatcatatcagaaagaatccagaaagctgatcctcaaggacctgagttaggagaagcttgtgaaaagggaaacatgttaaagaggcagagaataaagagagaaaagaaagatttcagacaagtgatagtgaatgactgtcacttacctgaaagcttcaaagaagaggaaaaccagaaatgtaagaaatctggaggaaaatatagccttaattctggcgctgttaaaaatccaaaaacccagcttggacaaaagccttttacgtgtagcgtgtgtgggaaaggatttagtcagagtgcaaacctcgttgtgcatcagcgaatccacactggagagaaaccctttgaatgtcatgagtgtgggaaggccttcattcagagtgcaaacctcgttgtgcatcagagaatccacactggacagaaaccttatgtttgctcaaaatgtgggaaagccttcactcagagttcaaatctgactgtacatcaaaaaatccactccttagaaaaaacttttaagtgcaatgaatgtgagaaagcctttagttacagctcacaacttgctcggcaccagaaagtccacattacggaaaaatgctatgaatgtaatgaatgtgggaaaacatttactaggagctcaaacctcattgtccaccagaggatccacactggggagaagccctttgcctgtaacgactgtggcaaagcctttacccagagtgcaaatcttattgtacatcagcgaagccatactggtgagaagccatatgagtgtaaagagtgtgggaaagcctttagttgtttttcacaccttattgtgcaccagagaattcacactgcagagaaaccttacgactgcagcgaatgtgggaaagccttcagtcagctctcttgccttattgtccaccagagaattcacagtggagatcttccttacgtgtgtaatgaatgtgggaaggccttcacatgtagctcatacctacttattcatcagagaattcataatggagaaaaaccttacacatgtaatgagtgtgggaaggccttcagacagaggtcgagcctcaccgtgcaccagagaacccacactggggagaagccctatgaatgtgagaagtgtggtgcagctttcatttccaactcacacctcatgcgacaccatagaacccatcttgttgaataacaagtaaggaagaggaagacctccagcattggtcataaccttctgcctccctaatgagacacctctttgctgttttcttcctcctctataaaagtgagggctgtgtccttaaaagttatagttttcaggaatgcagcagaagacacaagaaaagcatttcagaggctaatttaaaacaaaaagtaagcacctaaaggcaaggactttgttttaactgtaccttaagcggtcatgttctctgaagtggaatgtggctttcctaggaatgggtcgtacaaagctaagtggtaatgatgctatttggggaaaggtcttttttgcttaattttgttttttaaaactctgatgattgcttgagcaacaggcaggttatctgcctggttgaattctggttgaaccgtgtattctaatatttctggttaagtggtgactgggtaaggaaaccacttggggtagcagttcaacaattcacttacgaatgtttataagctttccatttcctaggtaattttttaaaagccagtcaaaacaaaaactttactgaaaatggacagaaataggaaatggactttttccttactgtctatacctcctgaaccttggtattgtaaagatctggggacctctgggtctgttctgaccattccctagtctccatggccaagcactcaaggattgatggacaccacacaccagctatattcatttgccaagatcaacagctccttctccaaacaactcaagcccccaattcccatcgcattccatttgggtgagatgcaactaacagccccttcctggatgaaggagtcaaagaataaagcttgcctagaggcatgcccca

SEQ ID NO: 31 - DNA, 2443 bp, homo sapiens
ccacgcgtccgggtagccacagcggcagctccagcccggcccggcagcgacatggaagatatacaaacaaatgcggaactgaaaagcactcaggagcagtctgtgcccgcagaaagtgcagcggttttgaatgactacagtttaaccaaatctcatgaaatggaaaatgtggacagtggagaaggcccagccaatgaagatgaagacataggagatgattcaatgaaagtgaaagatgaatacagtgaaagagatgagaatgttttaaagtcagaacccatgggaaatgcagaagagcctgaaatcccttacagctattcaagagaatataatgaatatgaaaacattaagttggagagacatgttgtctcattcgatagtagcaggccaaccagtggaaagatgaactgcgatgtgtgtggattatcctgcatcagcttcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctcacggggcatcttaggacacattctgtggagaaaccctacaaatgtgagttttgtggaaggagttacaagcagagaagttcccttgaggagcacaaggagcgctgccgtacatttcttcagagcactgacccaggggacactgcaagtgcggaggcaagacacatcaaagcagagatgggaagtgaaagagctctcgtactggacagattagcaagcaatgtggcaaaacgaaaaagctcaatgcctcagaaattcattggtgagaagcgccactgctttgatgtcaactataattcaagttacatgtatgagaaagagagtgagctcatacagacccgcatgatggaccaagccatcaataacgccatcagctatcttggcgccgaagccctgcgccccttggtccagacaccgcctgctcccacctcggagatggttccagttatcagcagcatgtatcccatagccctcacccgggctgagatgtcaaacggtgcccctcaagagctggaaaagaaaagcatccaccttccagagaagagcgtgccttctgagagaggcctctctcccaacaatagtggccacgactccacggacactgacagcaaccatgaagaacgccagaatcacatctatcagcaaaatcacatggtcctgtctcgggcccgcaatgggatgccacttctgaaggaggttccccgctcttacgaactcctcaagcccccgcccatctgcccaagagactccgtcaaagtgatcaacaaggaaggggaggtgatggatgtgtatcggtgtgaccactgccgcgtcctcttcctggactatgtgatgttcacgattcacatgggctgccacggcttccgtgaccctttcgagtgtaacatgtgtggatatcgaagccatgatcggtatgagttctcgtctcacatagccagaggagaacacagagccctgctgaagtgaatatctggtctcagggattgctcctatgtattcagcatcgtttctaaaaaccaatgacctcgcctaacagattgctctcaaaacatactcagttccaaacttcttttcataccatttttagctgtgttcacaggggtagccagagaaacactgtcttccttcagaaattattcgcaggtctagcatattattacttttgtgaaacctttgttttcccatcagggacttgaattttatggaatttaaaagccaaaaaggtatttggtcattatcttctacagcagtggaatgagtggtcccggagatgtgctatatgaaacattctttctgagatatatcaaccacacgtggaaaagcctttcagtcatacatgcaaatccacaaagaggaagagctgaccagctgaccttgctgggaagcctcacccttctgcccttcacaggctgaagggttaagatctaatctccctaatctaaatgacagtctaagagtaagtaaaagaacagccataaaataagtatctgttacgagtaactgaagaccccattctccaagcatcagatccatttcctatcacaacatttttaaaaaatgtcatctgatggcacttctgcttctgtcctttaccttcccatctccagtgaaaagctgagctgctttgggctaaaccagttgtctatagaagaaaatctatgccagaagaactcatggttttaaatatagaccatcatcgaaactccagaaatttatccactgtggatgatgacatcgctttcctttggtcaaggttggcagagcaagggtataaagggggaaattgtttggcagcaccaacagaaaacaaaacaaaaaaaaaaaaaaa

SEQ ID NO: 32 - DNA, 4076 bp, homo sapiens
ctcttcttcttaaacaaaccacaaacggatgtgagggaaggaaggtgtttcttttactcctgagcccagacacctcactctgttccgtctaagcttgttttgctgaacacttttttttaaaaaaggaaaaagaaaaggagttgcttgatgtgagagtgaaatggacgtaagattttatccacctccagcccagcccgccgctgcgcccgacgctccctgtctgggaccttctccctgcctggacccctactattgcaacaagtttgacggtgagaacatgtatatgagcatgacagagccgagccaggactatgtgccagccagccagtcctaccctggtccaagcctggaaagtgaagacttcaacattccaccaattactcctccttccctcccagaccactcgctggtgcacctgaatgaagttgagtctggttaccattctctgtgtcaccccatgaaccataatggcctgctaccatttcatccacaaaacatggacctccctgaaatcacagtctccaatatgctgggccaggatggaacactgctttctaattccatttctgtgatgccagatatacgaaacccagaaggaactcagtacagttcccatcctcagatggcagccatgagaccaaggggccagcctgcagacatcaggcagcagccaggaatgatgccacatggccagctgactaccattaaccagtcacagctaagtgctcaacttggtttgaatatgggaggaagcaatgttccccacaactcaccatctccacctggaagcaagtctgcaactccttcaccatccagttcagtgcatgaagatgaaggcgatgatacctctaagatcaatggtggagagaagcggcctgcctctgatatggggaaaaaaccaaaaactcccaaaaagaagaagaagaaggatcccaatgagccccagaagcctgtgtctgcctatgcgttattctttcgtgatactcaggccgccatcaagggccaaaatccaaacgctacctttggcgaagtctctaaaattgtggcttcaatgtgggacggtttaggagaagagcaaaaacaggtctataaaaagaaaaccgaggctgcgaagaaggagtacctgaagcaactcgcagcatacagagccagccttgtatccaagagctacagtgaacctgttgacgtgaagacatctcaacctcctcagctgatcaattcgaagccgtcggtgttccatgggcccagccaggcccactcggccctgtacctaagttcccactatcaccaacaaccgggaatgaatcctcacctaactgccatgcatcctagtctccccaggaacatagcccccaagccgaataaccaaatgccagtgactgtctctatagcaaacatggctgtgtcccctcctcctcccctccagatcagcccgcctcttcaccagcatctcaacatgcagcagcaccagccgctcaccatgcagcagccccttgggaaccagctccccatgcaggtccagtctgccttacactcacccaccatgcagcaaggatttactcttcaacccgactatcagactattatcaatcctacatctacagctgcacaagttgtcacccaggcaatggagtatgtgcgttcggggtgcagaaatcctcccccacaaccggtggactggaataacgactactgcagtagtgggggcatgcagagggacaaagcactgtaccttacttgagaatctgaacacctcttctttccactgaggaattcagggaagtgttttcaccatggattgctttgtacagtcaaggcagttctccattttattagaaaatacaagttgctaagcacttaggaccatttgagcttgtgggtcacccactctggaagaaatagtcatgcttctttattatttttttaatcctttatggacattgtttttcttcttccctgaaggaaatttggaccattcagattttatgttggttttttgctgtgaagtgctgcgctctagtaactgccttagcaactgtagatgtctcggataaaagtcctggattttccattggttttcataatgggtgtttatatgaaactactaaagactttttaaatggcttgatgtagcagtcatagcaagtttgtaaatagcatctatgttacactctcctagagtataaaatgtgaatgtttttgtagctaaattgtaattgaaactggctcattccagtttattgatttcacaataggggttaaattggcaaacattcatatttttacttcatttttaaaacaactgactgatagttctatattttcaaaatatttgaaaataaaaagtattcccaagtgattttaatttaaaaacaaattggctttgtctcattgatcagacaaaaagaaactagtattaagggaagcgcaaacacatttattttgtactgcagaaaaattgcttttttgtatcactttttgtgtaatggttagtaaatgtcatttaagtccttttatgtataaaactgccaaatgcttacctggtattttattagatgcagaaacagattggaaacagctaaattacaacttttacatatggctctgtcttattgtttcttcatactgtgtctgtatttaatctttttttatggaacctgttgcgcctatttatgaaataataaatataggtgtttgtaagtaaatttgttagtatttgaaagaggtttctttgatgttttaacttttgctggcaaaaaaaaattcacgcttggtgtgaatactttattatttagtttttacagtaacatgaataaagccaaacctgcttttcatttagcagcaaattaaagtaaccagtccttatttctgcatttctttggttgatgcaaacaaaaaactattatatttaagaactttatttcttcatacgacataacagaattgccctccaagtcacacaagctccaagactaaacaaacagacaggtcctctgtcttaaaaaggttacttcttggttctcagctggttctagtcaattctgaaccaccaccccccgccccccgcaaaaaagtaaaagtcaaaccaaacttcctcaagctgcatgcttttcacaaaatccagaaagcatttaagaattgaactaggggctggaagaagtgaaagggaagcatctaaaaatgaaaggtgagtaaccagatagcaaaagaaaagggaaagccatccaaatttgaaagctgttgatagaaattgagattcttgctgtcttttgtgcctctacaagctactactcattccagaattcctgggtcttccaagaggattcttaaggtaccagagatttgctagggaaccaaaagtgcttgagaatctgcctgagggcttgcatagctttcacattaaaaaaagaaaaagctagcagatttactcctttttaggggatcatatcaagaaagttagtctggttggaaaccaagagaatggctgatgtctctttcttggaatatgtgaaataaatttagcagtttaactaaatacaaatatatgcattgtgtaatccactcagaattaaacagacaaaaggtatgcttgctttggaatgattttaggcattgtacaaccttgaatcacttgagcatgtaataactaataaataatgcagatccatgtgattattaaaatgactgtagctgagagctctaattttcctgtcttgaaactgtataagaactcatgtgattaagttcacagtttattgtttgtctgtttagtattttagaaatataccagcactactaattaactaatgtcttttatttattatattatgataaagtaaaaatttcacttgcattaagtctaaactgagaaggtaattactgggaggagaatgagcagctttgactttgacaggcggtttgtgcaggaaagcacagtgccgtgttgtttacagcttttctagagcagctgtgcgaccagggtagagagtgttgaaattcaataccaaatacagtaaaaacaaatgtaaataaaagaaaacacatcatcaataaaactgttattatgcgtgaccgta

SEQ ID NO: 33 - DNA, 4340 bp, homo sapiens
attcattcattccccgtggcactggaggcggcccactctgctctgtcagcttcggagctcctccaccctggctgccgaaagccccttcccgccatctaatgatacactctgcatacgcttctgttgagaatttgtggctagacattcctgtgggaccgggaatccaaattcttgggtacaaacagaaacttactttccttggggatttttttctctctctcactcacacacactctcgcgttctttccttttttctttttcgtagcagcaggggggaaaaaagagacaaaaacaaaacaaaaaacaacaaaaagcaacaccccccccttttattttcaaaagtagctagaggaaaaaaaaataaaacaacagccaaccaagtgaatcccaacccaaccccctgaagggctgaaaattctcgccttcttcagagcggggcatggcatcgaacagcatcttcgactccttcccgacctactcgccgaccttcatccgcgacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactgaccgccctggtggactcctcccgctggaggcggggaccctaacaaccttcaagaccagtgatgggccggctccgaggctccgggcgggaatgggacctgcgctccagggtggtctcggtcccagggtggtcccagctggtgggagcctctggctgcatctgtgcagccacatccttgtacagaggcataggttaccacccccaccccggcccgggatactgcccccggcccagatcctggccgtctcatcccatacttctgtggggaatcagcctcctgccacccccccggaaggacctcactgtctccagctatgcccagtgctgcatgggacccatgtctcctgggacagaggccatctctcttccagagagaggcagcattggcccacaggataagcctcaggccctgggaaacctcccgacccctgcaccttcgttggagcccctgcatcccctgggtccagccccctctgcatttacacagatttgagtcagaactggaaagtgtcccccacccccaccaccctcgagcggggttcccctcattgtacagatggggcaggacccagcacgctgctggcagagatggtttgagaacacatccaagccagtccccccagcccagcttcccctccgttcctaactgttggctttcccccagccgcacgggtcccaggccccagagaagatgagtctatggcatcaggttcttaaacccaggaaagcacctacagaccggctcctccatgcactttaccagctcaacgcatccactctctgttctcttggcagggcgggggaggggggataggaggtcccctttcccctaggtggtctcataattccatttgtggagagaacaggagggccagatagataggtcctagcagaaggcattgaggtgagggatcattttgggtcagacatcaatgtccctgtcccccctgggtccagccaagctgtgccccatcccccaagcctcctgggaggatccagccaaatcttgcgactcctggcacacacctgtctgtaacctgttttgtgctctgaaagcaaatagtcctgagcaaaaaaaaaaaaaaaacaaaaaaacaaaaaaaaaacaaaacagtttttaaaactgattttagaaaaagaagcttaatctaacgttttcaaacacaaggtctcttacaggtatagttccgtgattatgatagctctgtgattataagcaacatccccgccccctctcccccccgcggacccccagctgcctcctgagggtgtggggttattagggtctcaatactttctcaaggggctacactccccatcaggcagcatcccaccagcctgcaccacaggctcccctgggaggacgagggaaacgctgatgagacgctgggcatctctcctctgtggctctaggacatctgtccaggaggctgggcggaggtgggcaggatgtgagaggtggggagtactggctgtgcgtggcaggacagaagcactgtaaagggctctccagccgcagctcagctgcactgcgttccgaggtgaagtcttgcccctgaattttgcaaaatgggaaagtgggcgcttgcccaagggccaggctgcatggattctcacatcagagttctctggccctagaaaggcttagaaaaggcgtaagggaactcataaaggctagcagcatgcggtattttaactttctgcctcggcctctgtggatgcagaaatctgccctacaaaatgctcttcattggttgtctctgtgagagcactgtccccacccaacctgtcacaacggccagaaccatacaccagagacacactggcaggttaggcagtccttctggtgatcctattccattccctcctgctgcggtttctcttggcctgtcctcactggaaaaacagtctccatctcctcaaaatagttgctgactccctgcacccaaggggcctctccatgccttcttaggaagcagctatgaatccattgtccttgtagtttcttccctcctgttctctggttatagctggtcccaggtcagcgtgggaggcacctttgggttcccagtgcccagcactttgtagtctcatcccagattactaacccttcctgatcctggagaggcagggatagtaaataaattgctcttcctaccccatcccccatcccctgacaaaaagtgacggcagccgtactgagtctgtaaggcccaaagtgggtacagacagcctgggctggtaaaagtaggtccttatttacaaggctgcgttaaagttgtactaggcaaacacactgatgtaggaagcacgaggaaaggaagacgttttgatatagtgttactgtgagcctgtcagtagtgggtaccaatcttttgtgacatattgtcatgctgaggtgtgacacctgctgcactcatctgatgtaaaaccatcccagagctggcgagaggatggagctgggtggaaactgctttgcactatcgtttgcttggtgtttgtttttaacgcacaacttgcttgtacagtaaactgtcttctgtactatttaactgtaaaatggaattttgactgatttgttacaataatataactctgagatgtgtggaagga

SEQ ID NO: 34 - DNA, 10832 bp, homo sapiens
attcatttcctgagaactgcagagagccgctgagaggctctgcgtgtgcgtgtgcgcgggtgacgccgtgtgtgtgcgagtgtgtgtgtgtgtgtgcgcgcgcgcgtgtgagagagagaaagggagagagagagggggactctgtgtgagggaaagaaaacaatttctcctgctctgcagcttctgttcaggatcaatgtgactctaagaacaaatggatgaatgaatatccatatgaagagaaaaacaataaagaatatcaacacctttgagaacagaatgttaatgcttgatgggatgccggcagtcagagtcaaaacagagcttttggaatctgaacaagggtctccaaacgtccacaactatcccgatatggaagccgttcccctgttgctaaataatgtgaaaggggagcccccggaggactcgttatctgtagatcacttccaaacacaaactgagccagtggacttgtcaataaacaaagccaggacgtcccctactgccgtttcatcctccccagtttccatgacagcatctgcctcctcaccttcttcaacttcaacctcttcatcgtcttctagtcgtctagcctcatccccaactgttatcacatcagtatcttcagcgtcatcttcgtcaacagtattaactccagggccccttgtggcctctgcatctggtgttggaggccagcagtttttgcacattatccatcccgtaccgccttcaagtcccatgaatttacagtctaacaaactgagtcatgttcaccgcatccccgtggtggtacagtcggtgcctgttgtctacacagctgtaaggtcacctggaaatgtgaacaacactattgtcgtgccgcttttggaggatgggagaggccatggcaaagcacaaatggacccccgaggcctatctcccagacaaagtaaaagtgacagtgatgatgatgacctgccaaatgtgaccttagatagcgttaatgaaactggatctacggccctttccatagccagagcagtacaagaggtacatccgtccccagtatcaagggtccggggcaatcgaatgaataatcaaaagtttccttgttcaatttcaccatttagtattgagagcacaagacgccagagacggtctgaatccccagactccagaaaacggcgtatccacagatgtgattttgagggatgcaacaaagtgtacacaaaaagttctcacctgaaggctcaccggaggacacatacaggagagaaaccttacaagtgtacctgggaaggctgcacctggaagttcgctcgttcagatgaactgacgaggcattaccgcaaacatacgggagtgaagccattcaagtgcgcggactgtgatcgcagcttttcccggtcagatcatttggccctgcaccgccggaggcatatgttggtgtgaggaatgctacctgtccagctgagcgtaagagctggatctcttagcggcacccaattcagcagggctgaatccccttcacagtgttaacacaaaagggcatcaccatcccacgatgtctgaaaccagagcaggaaaaagaaggcacacttcttttggtctgaaggtaacccccatcatgactagacgagaaccgtctttacccgggcctgggagctcagtgacataaatgctgaagagacaggcattgttttccgtgctgtgtcctctgccatttaaagatgtttgtagcttgtacattttctgagctgtcagacattttgttattgataccttaaaggtcacctaccacaaattgatggctagagcaagaccttttaaatttggatgaatctcccatcatcccatcccttaacccctttttacagaaattttagttacaatctgagtaagctagaacacacatccaatctgttaccagcaagcagcatgaaagtagaaacgaagaagcagatggagaggtcccattacctgcaaagaaagggcactcaggcagccctttgcaatcgcgatggcagccactagatatcactctcaacttgtcattctgccatgccctgaagaaaaccaacattgaatctttcatttgtttgtcgttgattgtttttgttttgtttcgattctgttttgttcatctgttcgagcagaggggcagttgaagtctcgtcctggtctctgccctggcatggactggcacagaggtgttctgtagttgaataggaagagcctgtctaaaaaactactgccccacttcaaattgcagtgttctgtcacctaggcatcatctcttcctgcccctagtatttgattacaaggaaccaggggaaaaaaactttcttagacacactggcaccaaggtaagaggtggggctgcccaggcaaagtcagtgaacatgaaaactcagacaaagcagagatggaaataatgcgcctcttgaggagaaaagcaataatgaataaaaggactttcctacaataacttcactgaggactcacgttaccaattttcatacttactaaagggattgtaaaaaacaccccagcattttaggtgtcttggttccatttacagcactgaggtaatctttctgctgtttgttgtcctgcttggttgagtaccacaattaaagattatgctccccttttcttgtttgaaaacagttatttggtgactagaaaggcaagggaggcatagccgggaattaactcttcggttaatgggtcagttctccttggaactgaatcagtggaaagagaatgcttcccatagaaagcccgacatggtgctagtttcctctcttggagagccaaggataagtgactctccaataggttcttctataccgatttcattttctaaactgtatcaggacccaggtgtcctgcattggcatctgcagggatattgaacccactcatcctgtgcagaggatgagaggaaggtgttagttccatcagccccataaaattatgaacctttttgcaactaggaacatatacagtaaacaagcaatacatcaccaacgagcattctttcagaaaaattaccatatttttgtccccaccacaagctgggtttttttccagctttctgtggctgaggggctggtgagattttttgttgttattgttgttgttgctagaattttttatagcttagatataaaaggtctccaacaaagacctttgcaatatattttaattacaaacacttgattttgggaattgcacatattaacctcacaatattactagctcatttttaaatgtctggacattctgaaataatttccggttacagaattaccctttttaggatcatcttctaattcagataatcttattctgatgaagcgagaactatgaacgttgaaacaaggacaatttgtgttggatggtattcaaaaggaattttttaaaacattaagcaacagtgtaatgattttcaggtcaaattatggcttcacaaatatgtgctctatattgtctgccaggtctcaaaaattcatcaagcaatttccctccaacaagggctgcagttctagtgataaatagcatctttagctgcacagtctactactcatcaaccagtgggttttttttagcacctggaaagtttttttgtactatatgcaactcctacttcacattgcctgtgaccattcagaatagaaaaaggtctgttgatgcatgccagtgctggttactagtcagtgacagagaacacagagagggacaaacacagccatgaaccccctggcactagtaccagcacctagttggcaagtagtgggtgcatctctcaagtgaagtgtagatttgtatttagtcctgcttgctctcataaaaagagccagttcttttctctgctgaaagatgtcttgagagtattacaggtagacttggtttttagaattcataaaactgacaggcaggggaatcttcctgagtgacagcttggtttaaatttagcacagaactagatgagttgcacttttctaataacataggatggatttggggaagggaacattttccctcatcctctgaaagtggaaaccagactgagatggtaagttaactgataaaacctgtcagcattccatcattttccccttttgacgtatcagtacttctaagaagggatctaagcacaattaagtgaattgagtccctggtctagcctcaggaactgaaacttctttggggcttgtcttgagacgtggcaaattctccttggtgagttttctaaaagtatttccccatggaaaggagaaagtggtttgggacagaaagaaatggaatgtcttcagtcatggaagagcctccatttcatctcatgtccatttctagcctctttcaaaaagcacagtgcatcatgagcaacagaaaagcagtagaatcctaaaggacataacagttgttccgtaactagaagcattctttgattttaaatttgaagctgttcagtttgcagctggggatatgtgcatggttgggaacttatacacatagagccattaactacttaagcagctgcagtagacctgattttctgtttgcagtatagagattgatttcaagggagttacattatttgaaaagttatgactttcagcctttgaggttttaagaatcccttttcatggtctgatcttaagttgtttgaaaaaagattcacgcttctgcatccacctctgccctcattctgtcaattgtgtgtggactctgagaagcctgcttgataacttgtcttaagttctattaaggcagttagactgagatttctaatgacttcaccataaagattatacttctgcctggcgtcgtttctataaaacactataggacatttgttttcaaaagcagttgagtaacagcaacataaaccacattttttaaaaaaatgagcacttttcattcacttgaaagcttaagtgggaaaaaggaagatgattgtcaggggaaaaacttacaaaatgctacagtgtttacaaatgccagttccaggtgattagaaaggccgttttcattatggttcagatttggaatagcttatgcaaagattgtgttcatttttatacaattctttttaactttaaattgccatctgtgttttaaacataattctgcactttggaatcagttcattagaatgataacatttatatggataaagatgcagtattccttattctgtacttgatttattatacttaccaaagctgcaactgaataaatctggtgaggtgaggccttcaaaatgtattaatacgcctttgttatgttgttatgactgcttagaaatgtagccatgatgttgttctaaagatgtcttaacatttatagtgaggattgacagaaattaagcagtgttaagaataccagcatccatgttacagtctgcgtataagggactgaatgtgaggtaactcttatgaatcataataggcccagaaagccttaatattcatagttcataatccagtctcagattttgctcttcaatgttgcctgtaaacttagcaggtaaatgtattctagtataaaacatcattgaccaaatctcttcctttagtttttctgaggtagtgcatcccattaaaaaagtctcttcaattagatttggctttagtttagaaaggggtttggtaagtcatgtcttccatatgacatttctactcatccccacactgccttccacttaacctgtacagacagccccaacacatgcacatgtgtgtacacaactgtatatgcagcagccagtgggctaacaaatatcagcaaagaacttttttaaaaagaatttgtaataatccgtgctgtctagaaatgtgagttacttaccttattaggaaaccggcttataatactatatatgactctgcctgcattttatagttaagaaatgtacataaaaattgctatattttcacagtttcattataatgtctgtttagtaacccatgtccactgatactttaaatatgctaattactgagcaattgcaatagaaatccagattttcataagaaaatgaaatagaaggatactgcatctttaaaaaatagagtgaagatattttactgtattcgtaacatatgtgatagtggaaaaagtattttcaaactcacaaattttacagaggttgtaaatagtttgatgaagtatgttgggaaagagagcttctagttttcatcacaataaaaagaaaaaacttgttcagatatgccttgtatatcttacacacaatgagtgaaattgccatgggaactctgtccaactttgtcagagcatgctcttcgtgttctgttcaaaatagagtggccacttgtcatggtttagccaggatggtttaaaatataatgaagtcattttaagttgcagacaactgattccaaactagtaatgtcttttcctgaaaagggcaaaacagctgcacaggattttgcttgtactaaagaggagcatcttacaattgttctcttttctcagttatctccctcctcaacagagtgtgaacaaccatacaggtgtttattcttgttttattccagggagactgcttttaggtataactacatgttgacaagattaagtgctgtggatatggccacgtgttaaaggaatctatactcgcaataggaataagctaatgcccattttcagaaataaattaaatgctgaaagtgcctgtcagtaaaccttatgaccaatgagatattcctatacttttacacagcaataattcttaataagactgggttttttcctgcacatgtgaacacacacacacatacacacagagagagtaaatacatacacatagtaaatcaaatctgttcacatgcccacaaaaaacgcttttatagctttcaaatatacctaatcttgtttacattaacatttcttaatttgatctttctaacttgcagaacgttaagaacacaaatctgaacattaagtttatcttcagtgaacactattgcatatggtaattataatttccagataaaaagttgactgtcatttgccccaaacttatgaaacagtatgtatgattcaaaaagttttgctcagttaaaaacaccaatttatttggcgcaaaagactgaaggatatttgtttgcttgttcattttatccactcatatcaaacgcacaaattcatctttacatttcttcattcccctttcccctaatatttcctattatcatcttaagaatcattttggaatgaatattgacagaatcatttctccccaaaaagtctgttgaagtaccactgacacttagtaagcctagcctttaaccatctttgcagttagtcctaaaaattaccagatgcacattcagaagctcacaggttttttcctgtaattactgtactgctaaaagtggagtcatggatagaaatatacaatgagactatatgaatagacatgaaccctggaaattctccaaccctgccatctcagagcacaaattcacccaaacaagctactgaggctatcagtaaaattatactcatggagagtcacacatcgggtaagggcaattttaaaagtaggactgtgattaaacattcaggctttagacttccatgatggactcagaccacaggcctcaaaaaaacatccactgttttctcctcccaatgttagaaagaagaaaagggagacggaaatgtaccaggataagggggtaaaataaccaattaagtgtcaaaggaaagctgctgggttccttaatctcactgaagtcaagtcttctgactgggcttcttaccatgtgttgtctgaaaaaactaaagtaccttgaaaggttacacattcagcaaaccatgaagataatagctattctttattaaacactgtgtgccaagcaatagactaggcaatttttagatacgttacctgcaacctgtacaacatttctacactttatggatgggaaacggagacatgggaagtgtggctgagttgttcatggatgtagaaatagtaaacggcagagtaggaaagtgaaaccgcctatctctgacctggaggtctgcctgtatctttcccactccaccacactgcacgtgggtgtcccgaaaccaccttcccagattcctgactctcagtaattttattatggacaacatgcatgagtagtcatcatatttttcaagtgaaatatcgggacatgatataacacatgacttaacaatggtactgaatatttgaaatcaggcctttcccggaaaatcatgcatgaaggatcattataaacaaacaatagcaaccagttgtctccccgaacttgtcacttttctcataaatgtctggcctggagctccaaaatcatccaaatacttagtagcattttagcctgagtacactttctcagttcctcaactctttgtatacctttccaccaatatagacattctagaatctgcttcagatgcatttgaaattttcacccccatggaactagtgattaatatcagagcccactcttgcagttggtaatggggtggcaatcaaacgttcagatgatgataaaggagagataatggataattctttttcagagttctcacttaacagctctgttgtggaatgttttaaatagtcttataaataatttgtttatagtattgttgttagtttaattgaattttatgtaagaagctgtccaacatcagagaaatgaaattcctcccactttctgtgtagaacaaggtctctgacagtattgattcatggaagtactaatggacttagaaaacattaagagaatgtcatttctcatagtgtttctgtttctgaaaatgaatctcctgaattattatctttctccctgttacttggctggggaaagagatagaagctgtataaacaaattctcttccatgctcaaagcaagtgttccatgtgcacaacctgctgcagactggggcccttctcagttaattgggtttcacaagcaataatttctccacaacaaaaaccacaacttgaagtgagttgaaaagagatcaatagtggaaacagtcgcctcagtactttttctttctggatttcatctctagaaatttgaagtgtttgagacagagtccaccctttgtgcaaggcgagaaccaatgaatggactccttgtgtgaattattgcatcttcttccaaagcaggttcatcaagactttcacagagattcatttttgttgagaagtaagggttaataggaggatagaatttggatccaaatctagtgataaaagtgtccaagcaatcaaaaagtaagatattttagggacataccaacatcttccctttctgctaatttcatgctccaaagatatggcaaaaaaaaaaatcataaaaagtgcttttgccctacttgtgttctagttttcccatggcagaattttgtaattacatccagaatatagtgtatattttgttcctcaaactttattacattggatggatattgttgaactggggcactggtgcctatattcaaggctctttcctatcaacgtgtctgtccacgatttgttgtgtttaaagcttcattttgaaaaatcactgtccccctgtggggtagtgactgtattgttttgttcatgtctatgtgggacacattgcatcacatggcaaaccaactctctgtggatgtgagataagtacttataaaaccagcttgaaaacatcgtcttatgtattatgtcatcctgcatcataatgcaattatgtgtatcataacatgctcatttaaaaaaagagaaaccagcaaattcatgtttgtccatagaagaatgtactcagaactttgtgttgtgaaacgatgagaacagaccacctttaagatacccacctgccacttaaaatgacttagttataattagtagtagtctagacgttgttcttggtgtgtgggggtcaattctaacgtcatgttcttttgaataaatctctcagtcatatttgaaaaaaaaatacatgggaataaagaaaaatatcatctttggccaaatcaagcaggcatcttttttcttttccttgacgtttagctcattatacgtggtgattggatcacgagatctgtccgtgtgaaaatacagaaacatcctttagtttacaaaacagttattctaggcttgaagcctctgaacagcaaattgaatagatgggctgcatctgatttgctttatggatgtaattttacaaaacactcttgggtctctgaccccagggagttaagagtgcccagaggaggtcctacacattaaaggataaagccccccagtgatgctggcaagcaaatgtgttgagttcttaaatcttcatttggttttctgttcaggattttaattgcaaatgaatttatttctccagtttatctaaagacctaatttcccaatagtttcctctgcatttatatactctgtagtgtttaggcaaccctgttataagtttattaatattatgtaagtgttgttcttgtatttatgtatagtgtatgtattgtaaatatactcagagcttttttccttttactgtaaaatggtgatttttttgccctatgataatgtaaagggagaccctcctaatgagattctctcagaggatgattattccagtctattctcagagatttaaatgaacaagtgttattgtttttaatggtgtctcagacatattctgttggtgcattgcttttctgtattcaactttcctatgaattgagctgtgaactgaaatagagtttaaacctttaactgtatgcatttgtataattatctgaatgaaggcatgaaggttaaataaagcattttgtatggaacaaaa

SEQ ID NO: 35 - DNA, 9265 bp, homo sapiens
agttctaacctgctctgcaggaataacggtcctgcctcccgacactcttggcgaggtttttgtacagtttgctccgggagctgtttcttcgcttccacctttttctcccccacacttcgcggcttcttcatgctttttcttctcaccatttctggccaaaactacaaacaagacttcgcagatcgagcctgcgtgctgccgaagcagggcgccgagtccatgcgaactgccatctgatccgctcttatcaatgaagcagccgatcatggcggatggcccccggtgcaagaggcgcaaacaagccaatcccaggaggaaaaacgtggtgaactatgacaatgtagtggacacaggttctgaaacagatgaggaagacaagcttcatattgctgaggatgacggtattgccaaccctctggaccaggagacgagtccagctagtgtgcccaaccatgagtcctccccacacgtgagccaagctctgttgccaagagaggaagaggaagatgaaataagggagggtggagtggaacacccctggcacaacaacgagattctacaagcctctgtagatggtccagaagaaatgaaggaagactatgacactatggggccagaagccacgatccagaccgcaattaacaatggtacagtgaagaatgcaaattgcacatcagattttgaggaatactttgccaaaagaaaactggaggaacgcgatggtcatgcagtcagcatcgaggagtaccttcagcgcagtgacacagccattatttacccagaagcccctgaggagctgtctcgccttggcacgccagaggccaatgggcaagaagaaaatgacctgccacctggaactccagatgcttttgcccaactgctgacctgcccctactgcgaccggggctacaagcgcttgacatcactgaaggagcacatcaagtaccgccacgagaagaatgaagagaacttttcctgccctctctgtagctacacgtttgcctaccgcacccagctcgagcggcatatggtgacacacaagccagggacagatcagcaccaaatgctaacccaaggagcaggtaatcgcaagttcaaatgcacagagtgtggcaaggccttcaaatataaacaccatctgaaagaacacctgcgaattcacagtggtgaaaaaccttacgagtgcccaaactgcaagaaacgtttctcccattctggttcctacagttcgcacatcagcagcaagaaatgtattggtttaatctctgtaaatggccgaatgagaaacaatatcaagacgggttcttcccctaattctgtttcttcttctcctactaattcagccattacccagttaagaaacaagttggagaatggaaaaccacttagtatgtctgaacagacaggcttacttaaaattaaaacagaaccactagacttcaatgactataaagttcttatggctacacacgggtttagtggcactagtccctttatgaatggtgggcttggagccaccagccctttaggagttcatccatctgctcagagtccaatgcagcacttaggtgtagggatggaagcccctttacttgggtttcccaccatgaatagtaatttaagtgaggtacaaaaggttctacagattgtggacaatactgtttccaggcaaaaaatggactgcaaggctgaagaaatttcaaagttgaaaggttatcacatgaaggatccatgctctcaacctgaggaacaaggagttacttctcctaatattccgcctgtcggtcttccggtagtgagtcataatggtgccactaaaagtattattgactatacgttggaaaaagtcaatgaagccaaagcttgcctccagagcttgactactgactcaaggagacagatcagtaatataaagaaagagaagctacgtactttaatagatttggtcactgatgacaaaatgattgagaaccacaacatatccactccattttcatgccagttctgtaaagaaagttttcctggccccatccctttgcatcagcatgaacgttacctttgtaagatgaatgaagagatcaaggcggtcctgcagcctcatgaaaacatagtccccaacaaagccggagtttttgttgataataaagccctcctcttgtcatctgtactttctgagaaaggaatgacaagccccatcaacccatacaaggaccacatgtctgtactcaaagcatactatgctatgaacatggagcccaactccgatgaactgctgaaaatttccattgctgtgggccttcctcaggaatttgtgaaggaatggtttgaacaacgaaaagtctaccagtactcaaattccaggtccccatccctggaaagaagctccaagccgttagctcccaacagtaaccctcccacaaaagactctttattacccaggtctcctgtaaaacctatggactccataacatcaccatctatagcagaactccacaacagtgttacgaattgtgatcctcctctcaggctaacaaaaccttcccattttaccaatattaaaccagttgaaaaattggaccactccaggagtaatactccttctcccttaaatctttcctccacatcttctaaaaactcccacagtagttcatacactccaaacagcttctcttctgaggagctccaggctgagcctttagacttgtcattaccaaaacaaatgaaagaacccaaaagtattatagccacaaagaacaaaacaaaagctagtagcatcagtttagatcataacagtgtttcttcctcatctgaaaactcagatgagcctctgaacttgacttttatcaagaaggaattttcaaattcaaataatctggacaacaaaagcactaacccagtgttcagcatgaacccatttagtgccaaacctttatacacagctcttccacctcaaagcgcatttccccctgctactttcatgccaccagtccagaccagtattcctgggctacgaccatacccaggactggatcagatgagcttcctaccacatatggcctacacctacccaactggagcagctacttttgctgatatgcagcaaaggagaaagtaccagcggaaacaaggatttcagggagaattgcttgatggagcacaagactacatgtcaggcctagatgatatgacagactccgactcctgtctgtctcgcaaaaagatcaagaagacagagagtggcatgtatgcatgtgacttatgtgacaagacattccagaaaagcagttcccttctgcgacataaatacgaacacacaggaaaaagaccacatcagtgtcagatttgtaagaaagcgtttaaacacaagcaccaccttatcgagcactcaaggcttcactcgggcgagaagccctatcagtgtgataaatgtggcaagcgcttctcacactcgggctcgtactcgcagcacatgaatcacaggtattcctactgcaagcgggaggcggaggagcgggaagcggcggagcgcgaggcgcgcgagaaagggcacttggaacccaccgagctgctgatgaaccgggcttacttgcagagcattacccctcaggggtactctgactcggaggagagggagagtatgccgagggatggcgagagcgagaaggagcacgagaaagaaggcgaggatggctacgggaagctgggcagacaggatggcgacgaggagttcgaggaggaagaggaagaaagtgaaaataaaagtatggatacggatcccgaaacgatacgagatgaagaagagactggagatcactccatggacgatagttcggaggatgggaaaatggaaaccaaatcagaccacgaggaagacaatatggaagatggcatgtaataaactactgcattttaagcttcctatttttttttccagtagtattgttacctgcttgaaaacactgctgtgttaagctgttcatgcacgtgcctgacgcttccaggaagctgtagagagggacagaaggggcggttcagccaagacagatgtagacggagttggagctgggtattgttaaaaactgcattatgcaaaaattttgtacagtgttaaggcctaaaaactgtgtggttcagagactaattcctgtgtttaatagcatttatactttaagcacaactagaaaattgtaagaattgcactctacttatgtatcactacaaactttaaaaaactatgtctaatttatattaatacattttaaaaaggtgcccgcactaccatacatcagtatttttattattattattgttattcctttttaatttaatgtgctcgcactacaatgcatcagtattatgattcctctgtactttcctttcgctattcatcaatttcccattttttttttcagcttaagtaaccacacaattttaggcctcaatttttttttttttctgtgaaggaacttgaagtgatgcatgtgtgaatttaagataccgaagtcttaaagtgacctggacgtgaaggaaaaagtaagatgagaaataaagaaagcctttgtaaggtggttttaaaagccttatatgcaaaccttttaatctgtgtttctgcaagtgccatccttgtacagtgttaagagggtaacatgggttacctttgcaccagcttcagtgttaagctcaccctgttctttgaagcacccatgtcagtattagaagaataggcagcagttccttagtttacatatgtttgtgcaattattttctgtacttttttgttcattaattttgtcagtattacaccaaactgtttttgcaacaaaaaaattttttttgcattcatttaattttaggtcaaataacattttatttatgtggctcattttatatttcctaattttatttatttcatactgtagtgtacagtattatagttcttcaatatatagatatattttagtaaaaaaggaacatgacgttgatcatttgggcaaattttacgtaaagagaagagcatttattgtgttttggaacattaattgtgagatgggatttttcaattttattattttatttttgtttttttccaattactggaaattccaaatttgggaacttttgatacgatcttgtgaaaacactgtattttcgactgaaaattccactttcttcatcttgttttttagctaaaaagagggactgttaaatacaatgtatgataccatgacaaaaatctttcctgaattgtctttgtaaaagtattattgaattttcaatttgtaatttcttttgaaaatgaccatgctcgaataaaaatgtagccaaactaagaatgtagttaatgagttctgtacttttagagagttttccttcaatgaccattaacatgtaacatgctttatgcttataataatgctaattatgtttttttcatataattttagtttagcaataattttgactggtaccaataactgttttttaaaattccatacctatgtacagcaattttacagcttttctcaactgatcctgattccagattgtgtatttttatgtgaggttatattattcaaatttagtctatttactttacagacatttctacttttgcattacgagtatttagagattatgtgttaaaaattcacttctctgtccaaggggtctttgtgatttattcaaaaaaaagtctaatttcaaaaagacagctattattcagtgttatttataatatgtaaccttttttaaaggattgggatagtttatctcactttttgaaatgcagacagtagtttaccgtttatctgaaactagaaggcgtgggtgggagaggaaaagctaaaagcaaatgctaacaaaaataaccgtgattttctaagacagtttttcagtttttacaagatgaccctaatattcagaatatgaatgtattcgtaggttttacataatgacttttatcaagaaactagattctgcttcttaaatctaattgccaagtgaagaataacagaaaaaacagattaccttatcaaatttacagctcttgaatatacagaactataatatagtagctgtccatgtattttttctactttagaatcaaagaagaaaagcatcattttgctattaaatttgctaaaattttgagtatgatatttccagttggcaagaacaacatatttatatttattccttagccataataccactttcctaaatttcacaaaagtcattctttgcaacttgaaactcaatagaaagtgtgtatgtgtgtgtgtgtatatatatatatatatacacacacacacatacacagaaaggatgtaatgaagatacagtaatagttgagcagacctttttagaaaaacatgtttttagctctatcttcaaactttctggcagagggggtggggggggcagggggaggagtggcatcaaaatgctatgcctcctgttatccacagcctagagtttttatatttggaaagtttagaaaattctatcctcgtttctccttctttgaatggcacaaataaatacactacataaatttttctggtttgaaaggctctaggcgataactttattaattcaacctgaaaatatcaagccattaaattttgtccgggtagaataaatccctgtggcctcttttaaagcaatgtaggtctctgttgcccatggggcatatctgtgtcccaatccacaagagataggaccaacaaacaatgaatgtgcaacctaactctttctccttggaaagaagaaagtgtgcacgaagtagaggagggtgggcagaccctgccttgcccctcctgttacccccttctctgtcatttgttcctaactccatttcataggcaggctcagaatacctgagtctgaaaatatcaggataacacttgtgaattgtgacaatcactacaatgtcccatatctgaggagttttttttaatgctatttatccgctggacacgattgcacattagggctgcataatcctctaactctagggaaaaataaaaacttttgatttgtcttaagattcttctccaaggtcgcaaacaagaaattcccctccacaaccaagagatgtgcattttagtaacatcagatgtgttcttctgttttatcaactacttactcttcccacacgcttagttctaaatctaacctttcccccctcgaatagggggcaggggaggatgaggaaacactggaacaactgaacacccctgcccattttctccaagagccttttgtattctagcatatctgtgcaatcttttcttttttcttcacatgacactgtaagcttaggcctgaaataactgggaagagagatgcgtatcagaatttctccgcaagagctaaacaaaacatacatcttccttagcatgaattggactgggggcggagtgggagggcttggaggaaaggggaaagaagggactatatttgaataaatatgaataaatgtattagatacttttcacaatcagataacttttaaaaaggtcattttttatctttctaataatgtaagccttaataaaagcaaatcttagtcacaaatttgaggagactgcccaataataagtttacatgtatttgaactgaaaaattgttaaccatgcttttgctccaagatgtgtgaggccattcaggggctgtagggccctggatatacacacaaacaagtgtgtgtatatctggagccccacacattgtaataaacacagctgcatttatttgactatgtgatcccatgtacatgtaaaaacattcaaacaaacacactcagcggatttatttattgtgcaatggggcaattattcaaataaacatgctcaatgcaattatttgaatctcacattgcatgttcatcaatcatagcactaaaaaaagagggggaaaaaacaccaaagaattcacatggggaaaaaatatatatatgaaaaccaccttattatagattttatagggcagctgaggttatggctcccttcttaactgtaactcaactattctgtattcaatgacatttgtttctaatgattaattggttcactcacttgatcatataatagcaaactttataaacctgtattgtgtagagatgtgaaatctctatatttcaagagcagaagagttctttctagacaccttacatcaagggacactggtccaattattatcgcttatataagcactcctataaattctgaaaaattttatacatgcaacaaaacattcctacatttgaagacattaagaaaaatcacaggtgactcatctgatcattctatatattaataaatattatgacatatatgtgaacacatcacaaatcatattggtgtaccaagaggcaatttatgcctctcttaagtatgtactgacataacctaatatactaaaatgggaaggggcttttagtcactgaaatatgcatcgtgtaacaaagatgaagaaaatacatggcttgtgcccatcataaaaaaagattcagactgaaggcttagctttggttttttcaattaaattgttaaactgtgcacagtgatttttttttagaacttgagacatttgtgatgttggctgtttaaatctttgttaccttcgctgtgaattgaaattgtacatatttagtaaatcatgcagacaaaacaaactttttagacaatatttttattggagagttttcttttcctgtatccatgttaaaaaaaaaaaagacctcctttcccaaaataaaaatgtcaatactaaatttaaagaagtataaaggaatgattgcttcctttagagcaaaatatttaaataaacatggagataattggcaacatgttctttttgggctagtaggctgtgtccaattttttgggtctgatgtttcagagggcctctgtttcagggttgaagatgatatattaatctcggaattaaacaaatgctattaaataacagaaataaatccttcagtcttcatttgtatctgaaatgtggaattagtcagagaaatggcaccgagataatatgatgctgaatatgccacaagctaaaactttctctattaaacgagtcgcctactacctataatctagcatgacatgttaaaatgctccaaaatactgaaaatagaaaatgaacactactgttccgatttggtcttgagattaatatgacaaagattctaataattttagtcaaagaaaatgatgagtaaaaaatacatgaagattaaagtgatgacaaatga

SEQ ID NO: 36 - DNA, 2257 bp, homo sapiens
ctctccttgttttgctttcgatctggactgttctcaggcaagccggggagtaacttttagttttgctcctgcgattattcaactgacgggctttcatttccatttcacataccctagcaacacttataccttgcggaattgtattggtagcgtgaaaaaagcacactgagagggcaccatgccggtggaaaggatgcgcatgcgcccgtggctggaggagcagataaactccaacacgatcccggggctcaagtggcttaacaaggaaaagaagatttttcagatcccctggatgcatgcggctagacatgggtgggatgtggaaaaagatgcaccactctttagaaactgggcaatccatacaggaaagcatcaaccaggagtagataaacctgatcccaaaacatggaaggcgaatttcagatgcgccatgaattccttgcctgatattgaagaagtcaaggataaaagcataaagaaaggaaataatgccttcagggtctaccgaatgctgcccctatcagaacggccttctaagaaaggaaagaaaccaaagacagaaaaagaagacaaagttaagcacatcaagcaagaaccagttgagtcatctctggggcttagtaatggagtaagtgatctttctcctgagtatgcggtcctgacttcaactataaaaaatgaagtggatagtacggtgaacatcatagttgtaggacagtcccatctggacagcaacattgagaatcaagagattgtcaccaatccgccagacatttgccaagttgtagaggtgaccactgagagcgacgagcagccggtcagcatgagcgagctctaccctctgcagatctcccccgtgtcttcctatgcagaaagcgaaacgactgatagtgtgcccagcgatgaagagagtgccgaggggcggccacactggcggaagaggaatattgaaggcaaacagtacctcagcaacatggggactcgaggctcctacctgctgcccggcatggcgtccttcgtcacttccaacaaaccggacctccaggtcaccatcaaagaggagagcaatccggtgccttacaacagctcctggcccccttttcaagacctccccctttcttcctccatgaccccagcatccagcagcagtcggccagaccgggagacccgggccagcgtcatcaagaaaacatcggatatcacccaggcccgcgtcaagagctgttaagcctctgactctccgcggtggttgttggggcttcttggctttgttttgttgtttgtttgtattttatttttttctctctgacacctattttagacaaatctaagggaaaaagccttgacaatagaacattgattgctgtgtccaactccagtactggagcttctctttaactcaggactccagcccattggtagacgtgtgtttctagagcctgctggatctcccagggctactcactcaagttcaaggaccaacaagggcagtggaggtgctgcattgcctgcggtcaaggccagcaaggtggagtggatgcctcagaacggacgagataatgtgaactagctggaattttttattcttgtgaatatgtacataggcagcactagcgacattgcagtctgcttctgcaccttatcttaaagcacttacagataggccttcttgtgatcttgctctatctcacagcacactcagcacccccttctctgcccattccccagcctctcttcctatcccatcccatcccatcccatcccatcccatcccatcccgctcttttcctacttttccttccctcaaagcttccattccacatccggaggagaagaaggaaatgaatttctctacagatgtcccattttcagactgctttaaaaaaaatccttctaatctgctatgcttgaatgccacgcggtacaaaggaaaaagtatcatggaaatattatgcaaattcccagatttgaagacaaaaatactctaattctaaccagagcaagcttttttattttttatacaggggaatattttattcaaggtaaaattctaaataaaatataattgttttttatcttttctacagcaaatttataattttaagattccttttcttgtttatcagcagttgttattacatccttgtggcacatttttttttttaattttgtaaaggtgaaaaaagcttttatgagctcatgtagcaatcagattttcctgtggattgataataaatgaatatgatatatagttaaa

SEQ ID NO: 37 - DNA, 3714 bp, homo sapiens
gcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggttgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctgggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcat

SEQ ID NO: 38 - DNA, 6255 bp, homo sapiens
actctaacaagtgactgcgcggcccgcgcccggggcggtgactgcggcaagccccctgggtccccgcgcggcgcatcccagcctgggcgggacgctcggccgcggcgaggcgggcaagcctggcagggcagagggagccccggctccgaggttgctcttcgcacccgaggatcagtcttggccccaaagcgcgacgcacaaatccacataacctgaggaccatggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaagctctttagaggaacataaagagcgctgccacaactacttggaaagcatgggccttccgggcacactgtacccagtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaaagccctcccgcgcccccaccccagaccccgagccaccccaggaaaagcacaaggactgccgccttctcgctcccgccagcagcatagactggactggaccagacaatgttgtgtttggatttgtaactgttttttgttttttgtttgagttggttgattggggtttgatttgcttttgaaaagatttttatttttagaggcagggctgcattgggagcatccagaactgctaccttcctagatgtttccccagaccgctggctgagattccctcacctgtcgcttcctagaatccccttctccaaacgattagtctaaattttcagagagaaatagataaaacacgccacagcctgggaaggagcgtgctctaccctgtgctaagcacggggttcgcgcaccaggtgtctttttccagtccccagaagcagagagcacagcccctgctgtgtgggtctgcaggtgagcagacaggacaggtgtgccgccacccaagtgccaagacacagcagggccaacaacctgtgcccaggccagcttcgagctacatgcatctagggcggagaggctgcacttgtgagagaaaatactatttcaagtcatattctgcgtaggaaaatgaattggttggggaaagtcgtgtctgtcagactgccctgggtggagggagacgccgggctagagcctttgggatcgtcctggattcactggctttgcggaggctgctcagatggcctgagcctcccgaggcttgctgccccgtaggaggagactgtcttcccgtgggcatatctggggagccctgttccccgctttttcactcccatacctttaatggcccccaaaatctgtcactacaatttaaacaccagtcccgaaatttggatcttctttctttttgaatctctcaaacggcaacattcctcagaaaccaaagctttatttcaaatctcttccttccctggctggttccatctagtaccagaggcctcttttcctgaagaaatccaatcctagccctcattttaattatgtacatctgtttgtagccacaagcctgaatttctcagtgttggtaagtttctttacctaccctcactatatattattctcgttttaaaacccataaaggagtgatttagaacagtcattaattttcaactcaatgaaatatgtgaagcccagcatctctgttgctaacacacagagctcacctgtttgaaaccaagctttcaaacatgttgaagctctttactgtaaaggcaagccagcatgtgtgtccacacatacataggatggctggctctgcacctgtaggatattggaatgcacagggcaattgagggactgagccagaccttcggagagtaatgccaccagatcccctaggaaagaggaggcaaatggcactgcaggtgagaaccccgcccatccgtgctatgacatggaggcactgaagcccgaggaaggtgtgtggagattctaatcccaacaagcaagggtctccttcaagattaatgctatcaatcattaaggtcattactctcaaccacctaggcaatgaagaatataccatttcaaatatttacagtacttgtcttcaccaacactgtcccaaggtgaaatgaagcaacagagaggaaattgtacataagtacctcagcatttaatccaaacaggggttcttagtctcagcactatgacattttgggctgactacttatttgttaggcgggagctctcctgtgcattgtaggataattagcagtatccctggtggctacccaatagacgccagtagcaccccgaattgacaacccaaactctccagacatcaccaactgtcccctgcgaggagaaatcactcctgggggagaaccactgacccaaatgaattctaaaccaatcaaatgtctgggaagccctccaagaaaaaaaatagaaaagcacttgaagaatattcccaatattcccggtcagcagtatcaaggctgacttgtgttcatgtggagtcattataaattctataaatcaattattccccttcggtcttaaaaatatatttcctcataaacatttgagttttgttgaaaagatggagtttacaaagataccattcttgagtcatggatttctctgctcacagaagggtgtggcatttggaaacgggaataaacaaaattgctgcaccaatgcactgagtgaaggaagagagacagaggatcaagggctttagacagcactccttcaatatgcaatcacagagaaagatgcgccttatccaagttaatatctctaaggtgagagccttcttagagtcagtttgttgcaaatttcacctactctgttcttttccatccatccccctgagtcagttggttgaagggagttattttttcaagtggaattcaaacaaagctcaaaccagaactgtaaatagtgattgcaggaattcttttctaaactgctttgccctttcctctcactgccttttatagccaatataaatgtctctttgcacaccttttgttgtggttttatattgtaacaccatttttctttgaaactattgtatttaaagtaaggtttcatattatgtcagcaagtaattaacttatgtttaaaaggtggccatatcatgtaccaaaagttgctgaagtttctcttctagctggtaaagtaggagtttgcatgacttcacactttttttgcgtagtttcttctgttgtatgatggcgtgagtgtgtgtcttgggtaccgctgtgtactactgtgtgcctagattccatgcactctcgttgtgtttgaagtaaatattggagaccggagggtaacaggttggcctgttgattacagctagtaatcgctgtgtcttgttccgccccctccctgacaccccagcttcccaggatgtggaaagcctggatctcagctccttgccccatatcccttctgtaatttgtacctaaagagtgtgattatcctaattcaagagtcactaaaactcatcacattatcattgcatatcagcaaagggtaaagtcctagcaccaattgcttcacataccagcatgttccatttccaatttagaattagccacataataaaatcttagaatcttccttgagaaagagctgcctgagatgtagttttgttatatggttccccaccgaccatttttgtgcttttttcttgttttgttttgttttgactgcactgtgagttttgtagtgtcctcttcttgccaaaacaaacgcgagatgaactggacttatgtagacaaatcgtgatgccagtgtatccttcctttcttcagttccagcaataatgaatggtcaacttttttaaaatctagatctctctcattcatttcaatgtatttttactttaagatgaaccaaaattattagacttatttaagatgtacaggcatcagaaaaaagaagcacataatgcttttggtgcgatggcactcactgtgaacatgtgtaaccacatattaatatgcaatattgtttccaatactttctaatacagttttttataatgttgtgtgtggtgattgttcaggtcgaatctgttgtatccagtacagctttaggtcttcagctgcccttctggcgagtacatgcacaggattgtaaatgagaaatgcagtcatatttccagtctgcctctatgatgatgttaaattattgctgtttagctgtgaacaagggatgtaccactggaggaatagagtatccttttgtacacattttgaaatgcttcttctgtagtgatagaacaaataaatgcaacgaatactctgtctgccctatcccgtgaagtccacactggcgtaagagaaggcccagcagagcaggaatctgcctagactttctcccaatgagatcccaatatgagagggagaagagatgggcctcaggacagctgcaataccacttgggaacacatgtggtgtcttgatgtggccagcgcagcagttcagcacaacgtacctcccatctacaacagtgctggacgtgggaattctaagtcccagtcttgagggtgggtggagatggagggcaacaagagatacatttccagttctccactgcagcatgcttcagtcattctgtgagtggccgggcccagggccctcacaatttcactaccttgtcttttacatagtcataagaattatcctcaacatagccttttgacgctgtaaatcttgagtattcatttacccttttctgatctcctggaaacagctgcctgcctgcattgcacttctcttcccgaggagtggggtaaatttaaaagtcaagttatagtttggatgttagtatagaattttgaaattgggaattaaaaatcaggactggggactgggagaccaaaaatttctgatcccatttctgatggatgtgtcacaccttttctgtcaaaataaaatgtcttggaggttatgactccttggtgaaa

SEQ ID NO: 39 - DNA, 5140 bp, homo sapiens
acttctcctttcgccggcgccgagttcctggcgccgctcgcccggcccggcttccgaggggagaggacgggctggcggggctggggacccgcgtctcggcccccggagcggggaccacggagacagaccccggcccggcgaccgagctgggcccgtgagccactcggcctcaggtcgctcctgtggttggtccagcccagaatgcagccttgagcctggcttaggccaccacctactccagctctctccaccccctattttactgcagctcagggggtaggctctaggctccaaagtacctgggtattgtcccttcatcaagaaagccccacagctctggagggctctgataatcccgttgtcagctctctgaaaagacagcatggctattaccctacagcccagtgacctgatctttgagttcgcaagcaacgggatggatgatgatatccaccagctggaagacccctctgtgttcccagctgtgatcgtggagcaggtaccctaccctgatttactgcatctgtactcgggactggagttggacgacgttcacaatggcatcataacagacgggaccttgtgcatgacgcaggatcagatcctggaaggcagttttttgctgacagatgacaatgaggccacctcgcacaccatgtcaaccgcggaagtcttactcaatatggagtctcccagcgatatcctggatgagaagcagatcttcagtacctccgaaatgcttccagactcggaccctgcaccagctgtcactctgcccaactacctgtttcctgcctctgagcccgatgccctgaacagggcgggtgacactagtgaccaggaggggcattctctggaggagaaggcctccagagaggaaagtgccaagaagactgggaaatcaaagaagagaatccggaagaccaagggcaaccgaagtacctcacctgtcactgaccccagcatccccattaggaagaaatcaaaggatggcaaaggcagcaccatctatctgtgggagttcctcctggctcttctgcaagacagaaacacctgtcccaagtacatcaagtggacccagcgagagaaaggcatcttcaaactggtggactccaaagctgtgtccaagctgtgggggaagcagaaaaacaagcctgacatgaactatgagacaatggggcgggcactaagatactactaccaaagaggcatactggccaaagtggaagggcagaggctggtgtaccagtttaaggagatgcccaaggacctggtggtcattgaagatgaggatgagagcagcgaagccacagcagccccacctcaggcctccacggcctctgtggcctctgccagtaccacccggcgaaccagctccagggtctcatccagatctgccccccagggcaagggcagctcttcttgggagaagccaaaaattcagcatgtcggtctccagccatctgcgagtctggaattgggaccgtcgctagacgaggagatccccactacctccaccatgctcgtctctccagcagagggccaggtcaagctcaccaaagctgtgagtgcatcttcagtgcccagcaacatccacctaggagtggcccccgtggggtcgggctcggccctgaccctgcagacgatcccactgaccacggtgctgaccaatgggcctcctgccagtactactgctcccactcagctcgttctccagagtgttccagcggcctctactttcaaggacaccttcactttgcaggcctctttccccctgaacgccagtttccaagacagccaggtggcagccccaggggctccactgattctcagtggcctcccccaacttctggctggggccaaccgtccgaccaacccggcgccacccacggtcacaggggctggaccagcagggcccagctctcagccccctgggactgtcattgctgccttcatcaggacttctggcactacagcagcccctagggtcaaggaggggccactgaggtcctcctcctatgttcagggtatggtgacgggggcccccatggaggggctgctggttcctgaagagaccctgagggagctcctgagagatcaggctcatcttcagccacttccaacccaggtggtttccaggggttcccacaatccgagccttctgggcaaccagactttgtctcctcccagccgccccactgttgggctgaccccagtggctgaacttgagctctcctcaggctcagggtccctgctgatggctgagcctagtgtgaccacatctgggagccttctgacaagatcccccaccccagcccctttctccccattcaaccctacttccctcattaagatggagccccatgacatataagcaaaggggtcagggcaagtgtgacccaccaggcaaaattgagcagcattttcatagggaccgacttcagtagcacacctgcccctgcatttcagtgggatgtcaatacacttgaccccaagtcccccggccctgcctggtgtcactgtggccaaacagtgcccagcttaagcatccctggcatcagactatggccttcaagagcactagggcatatgcttttggcagcataacgggctgacttggtgatggagggaaaaagccttgagccaggcagaagtttgtggccagggtttgtgcagcagctttgtgagaagagcccttctacctggctctatctcactggctgcattccctacacagggaatttactaccctatatgtgaatatccctgtatgtacttgtgtgtacttgttggtctgtatcttagtttctttggggaggacagggctgtagctgtgaggtcttgtctccaagggtgtgtgtatgtctccgtggatcagccacagggatagggattttgtttttaagggaaagcattctctaattccctttgttcatgccgagattcagttgctctgagactatggggtacaagtttgatcctccgaatctggagatgttgtagagctggaacgagtgcagagtaggaacgctttgatgcgcatgcacattggggaagatgcgctcctcagggacacaaaggccgagtggggtaaaaccacgaagggagggaagggaagtcagctctgggagcagccctcactggctggaccaaggtactcttcctggagtttgccgtgttagcaaccacagtcaccttgcagtcaggctggaatcttgggccaccccaaagtgctttgctgaaggatttagacggggatgaagtgccctccagcctcagagctagccacaaagcccccagagctgaattcattgagtatttgtgcctagggcttgggctgtttgtgtgataccggccccccgccagacaatagcctttgctgacaccccagcctacttccccgatcctgggctccctcttgattactttttgacattttccagctgtcaggcatcactgcggctagtccggcagcgacctagatggggtccacccccattcctgctcaagcatgggcacctaccacatggtttctgctgctcagcctgcctgcaactcacctcgaaggcggaccagcctgcctctgtgatgactgcagcagacctccttgggtgtaccaatgcccctcatctcccactttcacacctaaccctgactccttcaccaagaagacgggagtcggcagccaggagttcccgtggcacctctctctcttcgtggctccctgcttcccccttccctctttccgaggaagggtcaacctattctctctcaaagccaacccctaggccaattgcctggatctcctcccctctcccttctttaaacgagcttgcctccctcctgccaagtttgagggcaaggctaagaaatgtcagccacggaaacaactctattatctggtgactttgggtaatgtgaatcagtgcctgaggacctttgctgtgtccttggtacagaaccatccacttgacctaactacctcccctggccgcgctctcgctcttctcttctttgttaagccaacaactatcaccctctcctactcttcttctccctgccccctggagggcactgtgtttggttgtgcaaatgtatttactatgcgtgtttccagcagttggcattaaagtgcctttttctaataaaatcagtttattatgacagtttcctgatggttgaaagtaagcatcttgataaagggtcaccattaaaaaaaaattttgcataaaggtgctgcatgggttggggtagccccgcccccacctgaaaactggtttctgccacccctactccaactccatggaaactcattgctggaaggtcatcaatgacctcatggtgaaatcaaatgtcttcttcacagttctcgggcccccgtgagcccacactagctgggctctcctgcatcccccatcaccctttccggggctggttcttcacctaccacttccaacgtggctgttcaagaatctcatccattttgggctcattttggctcctcggagatgggtcctaaatctagagctccagtcccaacctttctcttaagctcctggctcacatttccagcaatctgctgaacatttccatgtgggtgcttgtcagctccttaaagatagcccctctatcaacaatgtttttgtttgttcgtttgtttttgagtcagagtcttgctctgtcggctggagtgcagtggtgcaatctcggttcactgcaacctctgcctcccaggctcaagtgattctcctgcctcagcctcctgagtagctgggagtacaggcacatgccaccatgcccagctaatttttgtattttcagtagagccagggtttcaccatttcggccaggctggtctcgaactcctggcctcaggtgatccacccaccttggcctcccaaagtgctgggattacaggcgtgagcaaccacggctggccaacaatggatttttaaattatttccacgctcacaaaaaacctttccaagatgacgggttgatgggtgcagcaaaccaccatggcacatgtatacctatgtaacaaacctgcacgttctgcacatgtatcccagaacttaaagtataataataataataataataataataataataataataaaaatcaagcaaacaaaaa

SEQ ID NO: 40 - DNA, 8494 bp, homo sapiens
agcagagaggagttgagggcgatgagagcgggtactgcgaactgccgggcgatgctgtcgctgccgccgtgatacggagagcaacagttccccagcaacacccctccccgacacaggcacacaccccccgacaggcacgcacacccaccccacagtgcccggctcggctgcgcctcctctattggcccaggaagcccacccagccccgccacgcagagcccagaaggaaagaaagcctcatgcctgagccgaggggagcaccatggatctgacaaaaatgggcatgatccagctgcagaaccctagccaccccacggggctactgtgcaaggccaaccagatgcggctggccgggactttgtgcgatgtggtcatcatggtggacagccaggagttccacgcccaccggacggtgctggcctgcaccagcaagatgtttgagatcctcttccaccgcaatagtcaacactatactttggacttcctctcgccaaagaccttccagcagattctggagtatgcatatacagccacgctgcaagccaaggcggaggacctggatgacctgctgtatgcggccgagatcctggagatcgagtacctggaggaacagtgcctgaagatgctggagaccatccaggcctcagacgacaatgacacggaggccaccatggccgatggcggggccgaggaagaagaggaccgcaaggctcggtacctcaagaacatcttcatctcgaagcattccagcgaggagagtgggtatgccagtgtggctggacagagcctccctgggcccatggtggaccagagcccttcagtctccacttcatttggtctttcagccatgagtcccaccaaggctgcagtggacagtttgatgaccataggacagtctctcctgcagggaactcttcagccacctgcagggcccgaggagccaactctggctgggggtgggcggcaccctggggtggctgaggtgaagacggagatgatgcaggtggatgaggtgcccagccaggacagccctggggcagccgagtccagcatctcaggagggatgggggacaaggttgaggaaagaggcaaagaggggcctgggaccccgactcgaagcagcgtcatcaccagtgctagggagctacactatgggcgagaggagagtgccgagcaggtgccacccccagctgaggctggccaggcccccactggccgacctgagcacccagcacccccgcctgagaagcatctgggcatctactccgtgttgcccaaccacaaggctgacgctgtattgagcatgccgtcttccgtgacctctggcctccacgtgcagcctgccctggctgtctccatggacttcagcacctatggggggctgctgccccagggcttcatccagagggagctgttcagcaagctgggggagctggctgtgggcatgaagtcagagagccggaccatcggagagcagtgcagcgtgtgtggggtcgagcttcctgataacgaggctgtggagcagcacaggaagctgcacagtgggatgaagacgtacgggtgcgagctctgcgggaagcggttcctggatagtttgcggctgagaatgcacttactggctcattcagcgggtgccaaagcctttgtctgtgatcagtgcggtgcacagttttcgaaggaggatgccctggagacacacaggcagacccatactggcactgacatggccgtcttctgtctgctgtgtgggaagcgcttccaggcgcagagcgcactgcagcagcacatggaggtccacgcgggcgtgcgcagctacatctgcagtgagtgcaaccgcaccttccccagccacacggctctcaaacgccacctgcgctcacatacaggcgaccacccctacgagtgtgagttctgtggcagctgcttccgggatgagagcacactcaagagccacaaacgcatccacacgggtgagaaaccctacgagtgcaatggctgtggcaagaagttcagcctcaagcatcagctggagacgcactatagggtgcacacaggtgagaagccctttgagtgtaagctctgccaccagcgctcccgggactactcggccatgatcaagcacctgagaacgcacaacggcgcctcgccctaccagtgcaccatctgcacagagtactgccccagcctctcctccatgcagaagcacatgaagggccacaagcccgaggagatcccgcccgactggaggatagagaagacgtacctctacctgtgctatgtgtgaagggaggcccgcggcggtggagccgagcggggagccaggaaagaagagttggagtgagatgaaggaaggactatgacaaataaaaaaggaaaagaaaaaaaaaaacagaaggaaaaggaaacctggtagctttttggccttggattctctctgggctccagatgacctggatgccaagccactgcccctcctctgggggcctccaccctcctctcccggctggaggtttgcctgattttctgggatggggttgggtattttgttcactcaaatggtggcacatttttctccttccttcacccagaccttcttttatgtgtccagaaatggaggctagaaagcaggtgagaggtcctctggtcagagccacacggtctgtgccggccttcctccaccaagacccccaggagatgaagggagggaggaggtgagccagaagggcgctcccctgctgatgggtctgggctgggtcactagctgctcaaaatggcagctctagtttgctggcggctgactggggaggagaagggctctgtttctcttcccaccactcagcccacctgctgctttcctctgctttgccacatctgggtgtcccccggtggtctctgagagcctcaggacccctgcccctcctccagctctttgttcccagcctccccttgactcctgtcctccaagtcccaggggagtcccagcccctcagggacctggcggtgtctccatccttctccggttccctagtgggatagctgcctctgacttcagggtccctcctaccctcagccttctgagaacacaaaacaaatccaataccccaccctagtccagcaccatcgtgagcaagattcctgctgcgctgcttccaaaatggaaaagcatcacaactaaatacctaatatcctaccaaacaaaccacagtccctacgtagccctacttcagtcccaggagaaggaatccatgaatgggctgaggttccgagggtaggttgtggatgtctttctctagaggggtcccacacctggaaaagaggacacccagcccgtgggatgagggaagctgggcaagggagaaagttgttgagtcacactggggaccccagatgtagtgagctcagcaagaaaagccaggtccagagagagtcgcacagaattcctgactcacagcgcccctcggtccacagtggagaggaggaggaggaggagcagaaggagggggaggagctgcaggaggaggaggatttgaaagcgctttggccttgtgttatgttttgcttcttttctgtgtcccctgttagcacattgtacttgaattacctcagttttttgtgacctcatgagctttcttaacctctgtatctctttttcggttggggcttggaggaatggggagggtgttcttttctgtttcttgtgtaaattaatagttgtttttatagacttcagctggtcaagaccctccccgtgcccctggagaccagcctcccagctggcaggaactgtgcaggaggagttgatactcaggatctgaatgtgagggccgaggagggcgaagagcgtgggtggggaggggatgttgctttggggtctcatggtccccagagggcactagggagtcacttttggctccgctggtgcatgaagtcacctgtggccaccatccgttccgcctaaaacactctggagagaatcccacctttcttacaatttgctgggccattgaggtttgagggcctgtgtttggatccttgcaactgtgtggtctcacctctgtgcgtgcattctcattgccagaattggtctattttcacggctggtctttggggaggggggcgggatccagcctttgttttgttgtgttggggtgggggtgttgtttttctaaaagctacctcttatgtttgtccagttgttcttttgttcctttcccgcctcccattctgctgctcttcctccctcttttccccaacctcccccgaccctcctgaattttggaaagcacatttgcaatattcgtgtttgctttgggacggaccctccgtttcatctcatgctttatgtgtaagagttttttattattattttttctttcctttctctctctgagaaagttgtggctgcgttttgatcttaggttttacaaagtggtttagggaagcggttttggggagaaggatcacgaggaatgtagggaagccggagggatgggtgctgctgcgacgacccccccgtccctcggccccagccctcctgccctccccttcaatctcatccaccaaatctgaaggccttaaaattgtgtgttgggaggatgtgaattgggaggacggtgtcactagactgtggattagggatggtaaagtagggaggatgctattttgcaactatagtaacgacttagtgttttggaaaggaaaagaagttaaacttgaaatacgtgactagaacagttgtcatgtttataatgtgaaaagggtgaaatcatttgggggaggggccgtctgtaagaaatcattatgcactatggcttcctcctctggtctgggaagaagcggggactggctggccctcaggggatctgtaaatcccagaaaacagtatttttaacagcaagatgtcattcaacattggtggggaaggaagagaaaaaaatcaaactattccatagaactagctggccccctcactcccatgcccttcccactcagcctgggcccctccccgctccattcataaaagctgagagggttgagctaatcttcacaaattgtaatatttttgtagtatctgttagttccttcgtcagttctgcagaaccttgccctttccttttgtaatgtgaataggaagacaaaagacaaaaaaaaatccaccaccaccaaaatatccctttgtacatgtatgtgcgtgtgcgcgtgtgctttgtgtgtgtggttgtgtgttaaatcatgcagtattgtcgtaatctggtgttgcagcaatggatggtactaaatcagcacctggatgccccacccaaccccgtgggccctgcagaccccagtagggaggtatggggagagctcaggggagtgtggtttctgagggctactgtctggggacacctctgaacttactgtaccttcctctccccatgaagacacctgaatagagtctaacatgcctcttctccaacttcctacctacaacaacagaacagttctaatgttgcacggcctagtggccagggggcaagctaagaggctgtctggaggctttatatgtgtctggagttaaggggagaggaggagggtagacaggggtctctccccaggtgggatctgaatatctgtcctcccctcttcttcatgccacctgactccttcggccccctggctgcctttagctgtggtactgctgacaaccctgcttgctactgccttatccagcacagtgaaaaacttctccagcctggcaaggccacgttggttaatagtccctttcccatgtccagctcctacaaatatgtcccttaatgcatttggtgacatttacacctcatgtgctctttccctattcactccttcactcattcaaagcattaaaatcctatgtatatataggatagacaaatatatagatatatagatatatatatatatagcaagagattgatataaaatagtaaatatcattgctgctttgggctgctttggaggaggaggccatgaatatggggaaggcagatctggggtgcaggggtaggtagggaggctgggggacccagtgattcagtacaatccaagggatgcaacgcgggcttgtttaatctttgtgcctgaacagtttttccatgttgagaaaaaaaagaaaaaaaaactgctgcaatttttcacaagtgtatggtacctttctggatgctattggtacaattgctgtggatggaagagggccattgagcttacctccctataggggagagagcagaggtggggcagcctttcgactctgtccatgacggctggcagggtcagggtagtttctgggcctgcttcccggcaagccgagctagggtgaaaactgggggcgcaccaggatgtgagacagaaaagcagaagatgagactctgttcattcacttttcctaggcccatcctgtggtcatctttccccctcccatcatacctcctccttcctggagcctctgccggcttggctgtaatggtggcacttacctggatatttcagtgggaggatgaaaggcgagactcaccctacgcggtgggacagatggggagaggaaaaaggcagagatggccaggagaggggtgcaggacaaaccagagaggttgggtcaggggaaaagggtggggagaaagaggggtgcaggccctgcaggccggttagccagcagctgcggcctccccgggcccttggcatccaacttcgcagacagggtaccagcctcctggtgtgtatcataggatttgttcacatagtgttatgcatgatcttcgtaaggttaagaagccgtggtggtgcaccatgacatccaacccgtatatataaagataaatatatatatatatgtatgtaaattatagcactgagggccctgctgccctgctggaccaagcaaaactaagccttttggtttgggtattatgtttcgttttgttatttgtttgtttttgtggcttgtcttatgtcgtgatagcacaagtgccagtcggattgctctgtattacagaatagtgtttttaattcatcaatgttctagttaatgtctacctcagcacctcctcttagcctaattttaggaggttgcccaattttgtttcttcaattttactggttacttttttgtacaaatcaatctctttctctctttctctcctccccacctctcacccttgccctctccatctccctctcccgccctcccctcctccctctggctccccgtctcatttctgtccactccattctctctccctctctcctgcctcctgctgccccctccccagcccacttccccgagttgtgcttgccgctccttatctgttctagttccgaagcagtttcactcgaagttgtgcagtcctggttgcagctttccgcatctgccttcgtttcgtgtagattgacgcgtttctttgtaatttcagtgtttctgacaagatttaaaaaaaaaaaaaaaggaaaaaaaaagaaaaaatgaatttactgctgcaggtttttttctctctccatgtgtcactaagtgaagtttgtgccttctatagcaaagagaatattttttacatcctactaacagtagatttttttgtagtgaacattttttgtatttttatttataagtctcataagaaaaatagcaatgttcagttgtataccttgaatctgcagttagaagagaataaagttaattcagttgtctctcgcttgaagcgtcccacctttttattgaagtacttggttttgttttgttttttttttttgttttttttgccttttcttgtgtggggaacatggtagaggaattaagatttttgtgtggggactgggaaaaagaaagctctgtattacacatatttagacttatctatgtgtcacttttatgccacatttacaaggcacagatgcactgaataacatttttctaatactttttagttaaaaaatgttaaatacgttaatatcgtatgatccttggtacaatgtgctaggtaggcacttgttcacttattcagcaagaccaccatgtaccagttactgttgtcggcgctgggaattacagcaaaggacaaacagacaaaccctggccgccctggggctaccatgctagcgggccgctgagtcggaatccgtggctgtgttaggataacttgctcaggattgcacggctggtgagcagcagtcagaactccatccacctctcctgataccaggccaggttcctttactccagagttctcaaagcaggggtctcacgtcttctttagtctttagagagacattttcaactgactccttagtggctaagcaagcttcagaaatttctcaacaagaagcttccacacaatcaaaggtccctgcctgcgtctgtagcagccaagagctgttaagaaaggagattggagcaggtggccttgtgcaggggaggggatgggtgggaggtggccgtgcaagctctgcattgtcatggacggaataaaggatttccacaaca

SEQ ID NO: 41 - DNA, 3470 bp, homo sapiens
actgggtcaagcacagccctgagcggccgcgtgtccgaggcccaggtgccctctagagccctgtagttcctgcccctctctgcccctctcggctcctgctgttccgccgctgtcgtccgaaccatcccaacccccagtccacccagacagcgcccgagctaggggagggaacggtctgggagtcggcagctggcgccagggcggccggaggatgccgaggggccggagccgggagggcccgaggccgaggcgcactctacccccagctcctaccctgtaagccccgccagcctccggacgtgctgtccctgggcccgtcgccctcggggctcccgccggaactccttcactctcagaggccgagtccctcccctccccacggctgcgtgtggccgttgccgtctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcctgccagcccggctcggcccagcaccccctctcctggagggcgcccagcagcctgccagcagttactgtgaatgttccccaccgctgagaggctgcctccgcacctgaccgctgcccaggtggggtttcctgcatggacagttgtttggagaacaacaaggacaactttatgtagagaaaaggaggggacgggacagacgaaggcaaccatttttagaaggaaaaaggattaggcaaaaataatttattttgctcttgtttctaacaaggacttggagacttggtggtctgagctgtcccaagtcctccggttcttcctcgggattggcgggtccacttgccagggctctgggggcagatttgtggggacctcagcctgcaccctcttctcctctggcttccctctctgaaatagccgaactccaggctgggctgagccaaagccagagtggccacggcccagggagggtgagctggtgcctgctttgacgggccaggccctggagggcagagacaatcacgggcggtcctgcacagattcccaggccagggctgggtcacaggaaggaaacaacattttcttgaaaggggaaacgtctcccagatcgctcccttggctttgaggccgaagctgctgtgactgtgtccccttactgagcgcaagccacagcctgtcttgtcaggtggaccctgtaaatacatcctttttctgctaacccttcaaccccctcgcctcctactctgagacaaaagaaaaaatattaaaaaaatgcataggcttaactcgctgatgagttaattgttttatttttaaactctttttgggtccagttgattgtacgtagccacaggagccctgctatgaaaggaataaaacctacacacaaggttggagctttgcaattctttttggaaaagagctgggatcccacagccctagtatgaaagctgggggtggggaggggcctttgctgcccttggtttctgggggctggttggcatttgctggcctggcagggggtgaaggcaggagttgggggcaggtcaggaccaggacccagggagaggctgtgtccctgctggggtctcaggtccagctttactgtggctgtctggatccttcccaaggtacagctgtatataaacgtgtcccgagcttagattctgtatgcggtgacggcggggtgtggtggcctgtgaggggcccctggcccaggaggaggattgtgctgatgtagtgaccaagtgcaatatgggcgggcagtcgctgcagggagcaccacggccagaagtaacttattttgtactagtgtccgcataagaaaaagaatcggcagtattttctgtttttatgttttatttggcttgttttattttggattagtgaactaagttattgttaattatgtacaacatttatatattgtctgtaaaaaatgtatgctatcctcttattcctttaaagtgagtactgttaagaataataaaatactttttgtgaa

SEQ ID NO: 42 - DNA, 3678 bp, homo sapiens
aatctacaggagccattttaacagctaaaacttgtcggattgctttttattttcaagctcaaaagacgatagagaaagaatacttgaaggccaagaagcttgagagaagaaaaatttcagaaaaattgtctcaatttgactagaatatcaatgaaccaggaaaactgaagcaccttccctaaagaaaacttgggtatacaattactccacagacagagctgagggttttttacccaaatcagtcactggattttgctgcctgatacgtgaatcttcttggaatttttctcatgtggatctaaggggaatgctttattatggctgctgttgtccaacagaacgacctagtatttgaatttgctagtaacgtcatggaggatgaacgacagcttggtgatccagctatttttcctgccgtaattgtggaacatgttcctggtgctgatattctcaatagttatgccggtctagcctgtgtggaagagcccaatgacatgattactgagagttcactggatgttgctgaagaagaaatcatagacgatgatgatgatgacatcacccttacagttgaagcttcttgtcatgacggggatgaaacaattgaaactattgaggctgctgaggcactcctcaatatggattcccctggccctatgctggatgaaaaacgaataaataataatatatttagttcacctgaagatgacatggttgttgccccagtcacccatgtgtccgtcacattagatgggattcctgaagtgatggaaacacagcaggtgcaagaaaaatatgcagactcaccgggagcctcatcaccagaacagcctaagaggaaaaaaggaagaaaaactaaaccaccacgaccagattccccagccactacgccaaatatatctgtgaagaagaaaaacaaagatggaaagggaaacacaatttatctttgggagtttttactggcactgctccaggacaaggctacttgtcctaaatacatcaagtggacccagcgagagaaaggcatttttaaattggtggattctaaagcagtgtccaggttgtgggggaagcacaaaaacaaacctgatatgaattatgagaccatgggaagagcactcaggtactattaccaaaggggtattctggcaaaagtggaaggtcagcgcttggtgtatcagtttaaagaaatgccaaaagatcttatatatataaatgatgaggatccaagttccagcatagagtcttcagatccatcgctatcttcatcagccacttcaaataggaatcaaaccagccggtcgagagtatcttcaagtccaggggtaaaaggaggagccactacagttctaaaaccagggaattctaaagctgcaaaacccaaagatcctgtggaagttgcacaaccatcagaagttttgaggacagtgcagcccacgcagtctccatatcctacccagctcttccggactgttcatgtagtacagccagtacaggctgtcccagagggagaagcagctagaaccagtaccatgcaggatgaaacattaaattcttccgttcagagtattaggactatacaggctccaacccaagttccagtggttgtgtctcctaggaatcagcagttgcatacagtaacactccaaacagtgccactcacaacagttatagccagcacagatccatcagcaggtactggatctcagaagtttattttacaagccattccatcatcacagcccatgacagtactgaaagaaaatgtcatgctgcagtcacaaaaggcgggctctcctccttcaattgtcttgggccctgcccaggttcagcaggtccttactagcaatgttcagaccatttgcaatggaaccgtcagtgtggcttcctctccatccttcagtgctactgcacctgtggtgaccttttctcctcgcagttcacagctggttgctcacccacctggcactgtaatcacttcagttatcaaaactcaagaaacaaaaactcttacacaggaagtagagaaaaaggaatctgaagatcatttgaaagagaacactgagaaaacggagcagcagccacagccttatgtgatggtagtgtccagttccaatggatttacttctcaggtagctatgaaacaaaacgaactgctggaacccaactctttttagttaatataccaaagcttatgaataattgtttgttaattgaacattttcaattatatgcagactgactgattctaagataaattctaaggaggtttctaattttgtaattgttaaaaatagagttaattttgactttgttagatgagggaggaaaactcaactgtttctctttgttatctaaatgtttcagaattcaatcgtgaaggaacaggcattttacactatgaagacattcttttgagatttttatttcagttgctatatcataagcatttttaaagtttcttttctaattttacattgtattagattttctgattcttttgtaaatacagaacttaaatagaaggcaacaggaaatttatataggaactattttcattccacttgtgtaagttaagtcttgactctttcaaatgcaaaaaacctattttatgctttgttaaaattatggtgtcacttagattgactttagttgactgcactatataatatagaactatgaatatgtagaataacatgaaaaattggaggtgctggtggtatggctgaccctgtttcagaagcaggatagtataaaagcatcagcctaagaatggcactcccactaactagctatgtaatcttgacctctttgggctttagttcctctcataaaaggaagagatgtattggattagactagatgatcaccactttctcttctagttctaatttttttaattctaatacctatattttcaagttatgtcaattaaatcattatcaggttatttcctaatgtaagaatagctaaaatgttgcagagaaataagtgacccaacaaaatttattcatctgttatgggtaagatctgccataaattcttcctaaataatttgtttactaactctttaggccactgtgctttgcggtccattagtaaacttgtgttgctaagtgctaaacagaatactgctattttgagagagtcaagactctttcttaagggccaagaaagcaacttgagccttgggctaatctggctgagtagtcagttataaaagcataattgctttatattttggatcattttttactgggggcggacttggggggggttgcatacaaagataacatatatatccaactttctgaaatgaaatgtttttagattactttttcaactgtaaataatgtacatttaatgtcacaagaaaaaaatgtcttctgcaaattttctagtataacagaaatttttgtagatgaaaaaaatcattatgtttagaggtctaatgctatgttttcatattacagagtgaatttgtatttaaacaaaaatttaaattttggaatcctctaaacatttttgtatctttaattggtttattattaaataaatcatataaaaattctcagtgtctgttttcaggcaaaagtttcttaaagaataagtgtgcagagaatattactagaacatcagcattacttaatgtttataaataaatttcattagtcagaa

(19) United States
(12) Patent Application Publication
(10) Pub. No.: US 2024/0018200 A1
(43) Pub. Date: Jan. 18, 2024

(54) COMPOSITIONS, CONSTRUCTS AND VECTORS FOR CELL REPROGRAMMING

(71) Applicant: Asgard Therapeutics AB, Lund (SE)

(72) Inventors: Inês Maria Nascimento Caiado, Vialonga (PT); Ilia Kurochkin, Lund (SE); Gladys Telliam Dushime, Malmö (SE); Ewa Sitnicka Quinn, Malmö (SE); Carlos Filipe Ribeiro Lemos Pereira, Moledo (PT)

(21) Appl. No.: 18/256,775

(22) PCT Filed: Dec. 17, 2021
(86) PCT No.: PCT/EP2021/086519

§ 371 Date: Jun. 9, 2023

(30) Foreign Application Priority Data
Dec. 17, 2020 (EP) 20215068.6

(51) Int. Cl. C07K 14/47; C12N 15/86; C12N 5/0783

(52) U.S. Cl. CPC C07K 14/4702; C12N 15/86; C12N 5/0646; C12N 2830/48; C12N 2310/20; C12N 2506/1307; C12N 2501/26; C12N 2501/60; C12N 2501/2315; C12N 2501/2307; C12N 2501/2303; C12N 2501/125

(57) ABSTRACT

The present disclosure relates to compositions, vectors, constructs, cells and methods for the reprogramming of cells into natural killer (NK) cells or progenitors. In particular, it relates to a combination of transcription factors for the reprogramming of cells.

### TECHNICAL FIELD

The present invention relates to the generation of autologous natural killer (NK) cells and progenitors by direct reprogramming using a combination of transcription factors. Furthermore, the present disclosure relates to the development of methods for making innate lymphocyte progenitors and mature cells with cytotoxic capacity from differentiated, multipotent or pluripotent stem cells by introducing and expressing isolated/synthetic transcription factors. In particular, the disclosure provides methods for obtaining autologous innate lymphocytes, particularly NK cells, by direct cell reprogramming using combinations of specific transcription factors.

### BACKGROUND

Cellular reprogramming is the process of changing the transcriptional and epigenetic networks of one cell state to that of a different cell type. Mouse and human somatic cells have been reprogrammed into induced pluripotent stem cells (iPSC) by the expression of defined transcription factors (TFs) (Takahashi et al., 2006). Additionally, somatic cells can be directly reprogrammed towards alternative somatic cell types. By using TFs with the ability to specify the identity of a target-cell, somatic cells have been directly converted into other mature cell fates, such as myocytes, neurons and hepatocytes (Pereira et al., 2012). In the hematopoietic system, direct reprogramming has been used to generate hematopoietic progenitors (Pereira et al., 2013, Gomes et al., 2018) and dendritic cells (Rosa et al., 2018) from mouse and human fibroblasts. Direct reprogramming is emerging as a powerful approach to study cell identity and commitment. Intrinsic lineage choice is stochastic and depends on the activation of lineage-specifying TFs that then lock in cell fate through cross-antagonistic interaction with alternative lineage-specifying factors (Graf and Enver, 2009). Despite of the recent success of lymphocytes in cancer immunotherapy, lymphoid cell generation by direct cell reprogramming has not been reported.

NK cells represent a distinct group of innate lymphoid cells (ILCs) controlling viral infections and cancer. Even though some NK cell generation is detected in the thymus, the bone marrow is the main site where NK cell progenitors and mature NK cells are produced in the adult (Vivier et al., 2011). NK cells are present in different tissues, such as peripheral blood, spleen and lymph nodes, as well as the liver, which is an active site for hematopoiesis and NK cell production during fetal life (Renoux et al., 2015). Several transcription factors are important for NK cell generation and function; however, many of them are also implicated in the regulation of other blood lineages. ETS1 expression is initiated in lymphoid progenitors and has been implicated in both early lymphoid commitment as well as NK terminal differentiation Together with NFIL3, ETS1 regulates NK cell transcriptional network, activating ID2 and EOMES expression. TBET and EOMES are both essential for NK cell functional properties, such as IFN-gamma production. Even though TBET is expressed by other ILCs, EOMES is restricted to NK cell lineage, and ectopic expression of EOMES in TBET⁺ cells induce NK-cell-like features. Despite the importance of these individual TFs in NK biology, the key combination that instruct NK identity in other cell-types is unknown. The TFs RORα, SMAD7, FOS, JUN and NFATC2, have been suggested to have such a role based on computational predictions (WO 2019/177936). Overall, the successful generation of NK cells by direct cell reprogramming has not been reported.

Unlike T cells, NK lymphocytes can directly recognize and eliminate target cells without requiring antigen-specification. Instead, NK cell killing depends on a balance of signals from activating and inhibitory receptors at the NK cell surface (Noriko et al., 2020). Because they are inhibited by Major Histocompatibility Complex (MHC) class I molecules, NK lymphocytes eliminate tumor cells that downregulate MHC-I at surface without reacting with healthy tissues, supporting their clinical utility. Pre-clinical data have extensively demonstrated antitumor activity of NK cells, providing evidence of their ability to eliminate MHC-I negative cancer cells that are not targeted by T cells, increase immune cell infiltration (Bottcher et al., 2018, Barry et al., 2018) and importantly to prevent metastasis by eliminating circulating tumor cells (Lopez-Soto et al., 2017). Furthermore, the cytokine secretion profile of NK cells differs from T lymphocytes, which produce proinflammatory cytokines associated with the onset of cytokine release syndrome. Hence, NK-based immunotherapies have a safer profile than adoptive T cell strategies (Lopez-Soto et al., 2017). Clinical studies with adoptive transfer of NK cells isolated from peripheral blood or differentiated from umbilical cord hematopoietic progenitors have shown to be safe and demonstrate efficacy against hematopoietic cancers (Bachanova et al., 2014, Passweg et al., 2004). However, primary NK cells are difficult to obtain from peripheral blood or umbilical cord blood in large numbers. The NK cell line NK92 has also been tested in clinical trials with minimal side effects, although the need to irradiate these cells limits NK92 cells persistence after injection (Tam et al., 2003). In solid tumors, the immune suppressive microenvironment inhibits NK cell infiltration and activation. However, several strategies have been developed to harness NK cells to maximize their infiltration and antitumor activity in solid cancers. Engineering NK cells with chimeric antigen receptors (CAR) is being developed to direct NK lymphocytes towards tumor cells with specific antigens, increasing NK cell antitumoral activity. In preclinical studies, allogenic iPSC-derived CAR NK cells demonstrated the ability to prevent tumor progression and to promote sustained long-term survival in an ovarian xenograft model (Ye Li et al., 2018). Taken together, these studies demonstrate that primary and iPSCs-derived allogenic NK cells are safe and causing minimal graft-versus-host disease. Nevertheless, protocols to differentiate NK cells from iPSCs are complex and time-consuming, requiring addition of multiple cytokine combinations and co-culture with feeder layers. These limitations challenge the fast manufacturing and timely delivery of autologous iPSCs-NK cells, and although allogenic NK cells have proven to be safe, they can be rejected by the recipient's immune system, showing limited persistence (WO 2019/177936). MHC-I gene editing has been proposed as an alternative approach; however, MHC-I negative cells can be rejected by host NK cells and NK cells require cell-intrinsic MHC-I to maintain their function (Boudreau et al., 2016).

Therefore, novel approaches to allow the straightforward generation of NK cells that can be simultaneously genetically engineered are needed and hold compelling value for cancer immunotherapy.

### SUMMARY

The inventors of the present invention have developed a novel method of generating NK cells, which is fast and reproducible. In particular, the method involves generating autologous NK cells, which are have a high persistency in vivo, making them suitable for use in immunotherapy. The invention also relates to a combination of transcription factors, which have been shown to be enriched in NK cells, that can be used to reprogram cells into NK cells.

In one aspect, the present invention provides at least one polynucleotide encoding a combination of at least three different transcription factors selected from the group consisting of: ETS1, TBET, NFIL3, EOMES, ID2, GATA3, ZFP105, IKZF3, ETS1, TOX, RUNX3, KLF12, ZEB2, IRF2, STAT5, IKZF1, ELF4, ZBTB16, GATA2 and ELF1.

In another aspect, the present invention relates to at least one polynucleotide encoding a combination of at least three different transcription factors selected from the group consisting of: ETS1, TBET, NFIL3 and EOMES.

In one aspect, the present invention regards a construct or vector comprising the at least one polynucleotide as described herein.

In one aspect, the present invention provides a cell comprising the at least one polynucleotide, and/or the construct or vector as described herein.

In one aspect, the present invention relates to a pharmaceutical composition comprising the at least one polynucleotide, the construct or vector and/or the cell as described herein.

In one aspect, the present invention provides a method for reprogramming or inducing any cell into a natural killer cell or progenitor, the method comprising the following steps:
a. transducing a cell with the at least one polynucleotide, or the construct or vector as described herein;
b. culturing and expanding the transduced cell in a cell media; and
c. obtaining a reprogrammed cell.

In one aspect, the present invention provides an induced natural killer cell obtained by the method as described herein.

### DESCRIPTION OF DRAWINGS

FIG. 1. Schematic representation of the applications of directly reprogrammed NK cells. Human somatic cells, including fibroblasts, pluripotent stem cells, hematopoietic stem cells and other cell types isolated from patients are reprogrammed into autologous NK cells that can be applied for personalized immunotherapy. This direct reprogramming approach allows for gene editing to be done at the same time as transduction with TFs instructors of NK cell identity to improve NK cell activity or targeting. Induced NK cells may be expanded in vitro and employed towards the initiation of cytotoxic immune responses against cancer and viral infections.

FIG. 2. Identification of 19 candidate transcription factors to program NK cells. Heatmap showing gene expression profiles of the selected 19 TFs across multiple mouse tissues and cell types (GeneAtlas MOE430). Candidate TFs are highly enriched in NK cells when compared with more than 75 other tissues or cell types.

FIG. 3. Combined enforced expression of 19 transcription factors activate a NK-specific reporter. (A) Experimental strategy to screen NK-inducing TFs. NCR1-tdTomato double transgenic mouse embryonic fibroblasts (MEFS) were co-transduced with lentiviral particles encoding candidate TFs and M2rtTA and cultured in the presence of Dox for 12 days. tdTomato expression was monitored by Fluorescence Microscopy. (B) Gene Expression profile of NCR1 across several mouse tissues and cells (GeneAtlas MOE430). (C) Fluorescence Microscopy pictures of MEFS transduced with M2rtTA only (Left) or co-transduced with M2rtTA and all 19 candidate TFs (Right), 6 days after adding Dox. The entire well of a 6-well plate was acquired in an automated Zeiss CD7. A tdTomato positive colony generated with the combined expression of all TFs is highlighted at higher magnification at day 6. Scale bar=200 μm. (D) Number of tdTomato cells per colony between day 3 and day 12. Mean+SEM of thirteen colonies is shown.

FIG. 4. Number of tdTomato positive colonies increase with a restricted pool of transcription factors. (A) MEFs were transduced with control M2rtTA or co-transduced with pools of TFs (TF pool A-D). Quantification of tdTomato positive colonies per TF combination at day 6 (Left) and day 12 (Right). Mean+SD of two independent experiments are shown. p<0.05. (B) Fluorescence Microscopy pictures of MEFs transduced with M2rtTA (left) or co-transduced with M2rtTA and ETS1, NFIL3, ID2, TBET and EOMES (right), 6 days (top) and 12 days (bottom) after adding DOX.

FIG. 5. tdTomato positive colonies expand in culture. (A) Fluorescence Microscopy pictures of tdTomato positive colonies after transduction with ETS1, NFIL3, ID2, TBET and EOMES, 6 (Top) and 12 days (Bottom) after adding DOX. (B) Number of tdTomato cells per colony between day 3 and day 12. Mean+SEM of ten colonies is shown. (C) Flow cytometry plots and (D) quantification of tdTomato positive cells at day 12 after adding DOX. Mean±SD of two independent experiments.

FIG. 6. ETS1, NFIL3, TBET and EOMES combination increase reprogramming efficiency. MEFs were transduced with control M2rtTA, co-transduced with ETS1, NFIL3, ID2, TBET and EOMES or additional combinations where one transcription factor was individually removed from the 5 TF pool. (A) Number of tdTomato positive colonies quantified by immunofluorescence and (B) percentage of tdTomato positive cells quantified by flow cytometry at day 12 of reprogramming (Mean±SD; n=2).

FIG. 7. ETS1, NFIL3, TBET and EOMES are required and sufficient to efficiently activate NCR1-reporter. MEFs were transduced with control M2rtTA, co-transduced with ETS1, NFIL3, TBET and EOMES or additional combinations where one transcription factor was individually removed from the 4 TF pool. (A) Number of tdTomato positive colonies quantified by immunofluorescence and (B) percentage of tdTomato positive cells quantified by flow cytometry at day 12 of reprogramming (Mean±SD; n=2).

FIG. 8. The combined expression of ETS1, NFIL3, TBET and EOMES is enriched in NK cells. Gene expression enrichment score for the combination of ETS1, NFIL3, TBET and EOMES among 96 mouse tissues and cell-types. Gene expression data (GeneAtlas MOE430) log transformed and normalized to a 0-1 range for each gene followed by a search for highest average expression for ETS1+NFIL3+TBET+EOMES.

FIG. 9. RORα, SMAD7, FOS, JUN and NFATC2 combination does not activate NCR1-tdTomato reporter. The transcription factor combination of the present invention (ETS1, NFIL3, TBET and EOMES) was compared with a combination disclosed in Vivier et al., 2011 (RORα, SMAD7, FOS and JUN). The ability of the combinations to activate the NCR1-tdTomato reporter was analyzed. (A) Number of tdTomato positive colonies after transduction with RORα, SMAD7, FOS, JUN and NFATC2 combination or ETS1, NFIL3, TBET and EOMES combination at day 6 (Left) and 12 (Right) of reprogramming. M2rtTA-transduced MEFs were included as controls. (B) Gene expression enrichment score for the combination of RORα, SMAD7, FOS, JUN and NFATC2 among 96 mouse tissues and cell-types. Gene expression data (GeneAtlas MOE430) log-transformed and normalized to a 0-1 range for each gene with a followed by a search for highest average expression for RORα, SMAD7, FOS, JUN and NFATC2.

FIG. 10. Combined expression of ETS1, NFIL3, TBET and EOMES induces global gene expression changes. NCR1-tdT+MEFS were FACS sorted at day 3, 6 and 12 of reprogramming and profiled by single-cell RNA sequencing (RNA-seq) with 10× Genomics. Untransduced MEFs were included as control. t-SNE visualization of MEFS and reprogrammed cells at day 3, 6 and 12 (left). Expression of the mouse NK-specific marker Itga2 (right). The arrow indicates reprogramming trajectory.

FIG. 11. Fibroblast-associated genes are downregulated during NK reprogramming. t-SNE plots showing gene expression heat maps for the fibroblast-associated genes Col1a2, Lox and Fbnl2 in MEFS and reprogrammed cells at day 3, 6 and 12.

FIG. 12. ETS1, NFIL3, TBET and EOMES transcription factors induce natural killer transcriptional network. Quantification in violin plots of endogenous expression of 19 candidate transcription factors (TFs) (left) and Ets1, Nfil3, Tbx21 (encoding Tbet) and Eomes (right) in MEFS and during the reprogramming process.

FIG. 13. An Immature NK cell program is specified by enforced expression of ETS1, NFIL3, TBET and EOMES. (A) t-SNE plot of single-cell transcriptomes showing immature (left) and mature (right) NK gene signatures (Bezman et al. 2012) on NCR1-tdT+MEFs, 3, 6 and 12 days after transduction with the 4 transcription factors. (B) Whisker box plot showing expression distribution of immature and mature NK gene signatures in single-cells during NK reprogramming. (C) t-SNE gene expression heat maps for the NK-progenitor genes Cd38, Cd34 and Mme (encoding CD10) during reprogramming.

FIG. 14. ETS1, NFIL3, TBET and EOMES induce the expression of NK chemokines and cytokine receptors. Quantification in box plots ccl5, Ifih1 and II15ra in MEFS, day 3, 6 and 12 of reprograming.

FIG. 15. Schematic representation of experimental protocol to induce NK-like cells from human cells. Human embryonic fibroblasts (HEF) were co-transduced with lentiviral particles encoding M2rtTA (UbC-M2rtTA) and Ets1 (tetO-Ets1), Nfil3 (tetO-Nfil3), Tbet (tetO-Tbet) and Eomes (tetO-Eomes). Alternatively, a constitutive lentiviral driven expression with the SFFV promoter (SFFV-Ets1, SFFV-Nfil3, SFFV-Tbet and SFFV-Eomes) was tested. After overnight incubation with lentiviral particles, media was replaced with fresh growth media. For FUW-TetO transduced cells, growth media was supplemented with Doxycycline (Dox). A cytokine cocktail including stem cell factor (SCF), FMS-like tyrosine kinase 3 ligand (FLT3L), Interleukin-3 (IL-3), IL-7 and IL-15 was added to culture when indicated. NK reprogramming was assessed by flow cytometry, 6 and 12 days after transduction.

FIG. 16. Enforced expression of ETS1, NFIL3, TBET and EOMES in human fibroblasts generate CD34-positive cells. (A) Representative flow cytometry plots of human embryonic fibroblasts (HEFs), 6 and 12 days after transduction with the 4 transcription factors individually cloned into inducible (FUW-TetO) or constitutive (SFFV) lentiviral vectors. FUW-M2rtTA and an empty vector (SFFV-MCS) were used as controls. (B) Quantification of CD34+ cells induced with the 4 TFs with the two lentiviral expression systems at day 6 and 12 (Mean+SD, n=2).

FIG. 17. Cytokine signaling and enforced expression of transcription factors increase reprogramming efficiency. (A) Representative flow cytometry plots of human embryonic fibroblasts (HEFs), 6 and 12 days after transduction with the 4 TFs, with or without addition of the cytokines SCF, FLT3L, IL-3, IL-7 and IL-15. SFFV-MCS was used as control. (B) Quantification of CD34+ cells induced with 4 TFs in the presence or absence of the cytokine cocktail (Mean+SD, n=2).

FIG. 18. Combined expression of ETS1, NFIL3, TBET and EOMES induce human CD56-positive cells. (A) Representative flow cytometry plots of human embryonic fibroblasts (HEFs), 6 days after transduction with the 4 TFs. An empty vector (SFFV-MCS) was used as control (B) Quantification of CD56+ cells induced with 4 TFs (Mean+SD, n=2).

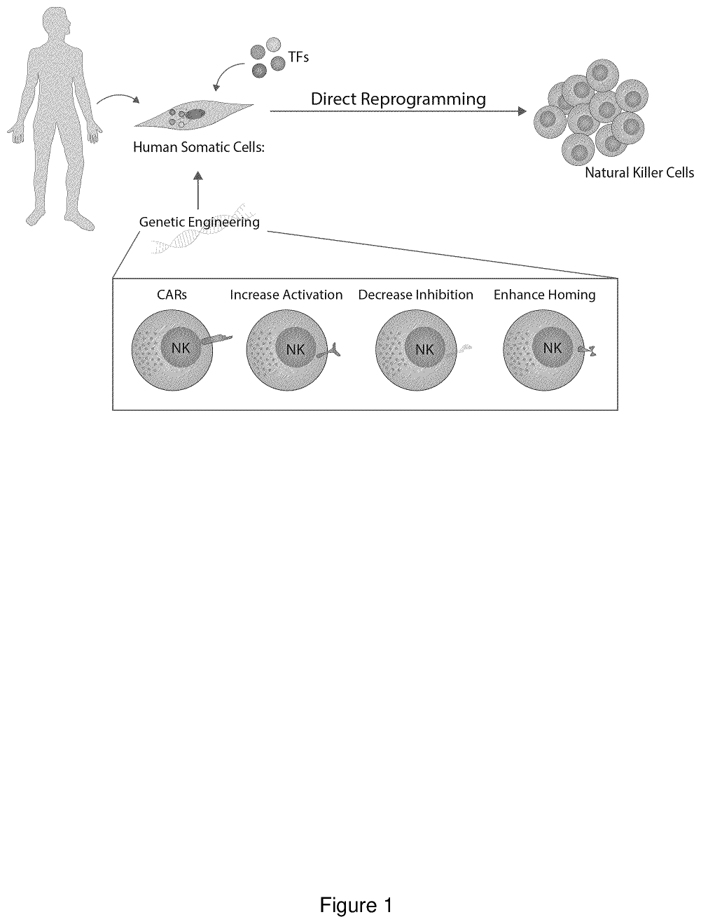

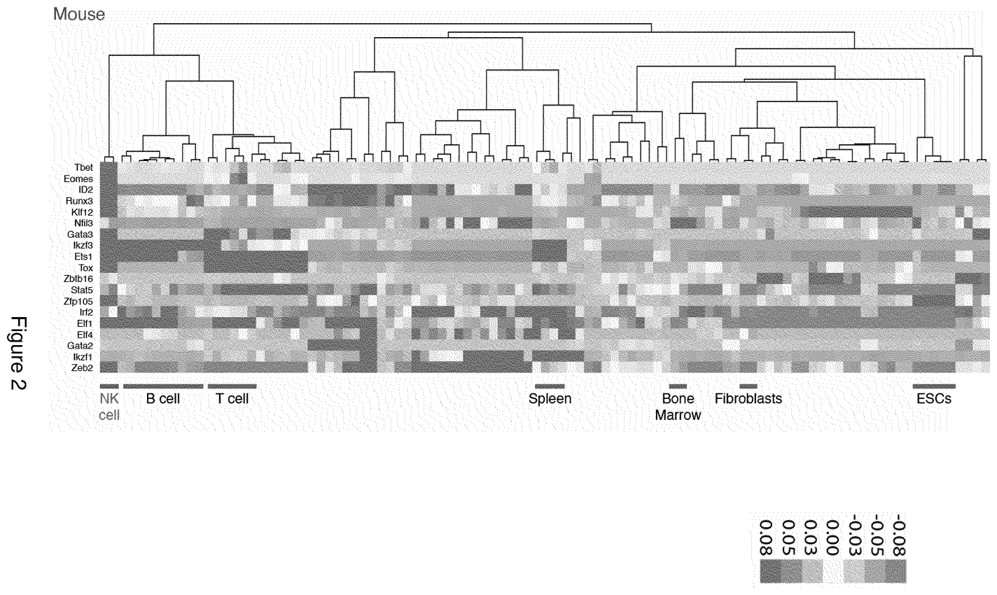

### DETAILED DESCRIPTION

The inventors of the present disclosure have identified a number of transcription factors capable of inducing a natural killer (NK) cell fate. They have also developed a novel method of reprogramming cells into NK cells using at least three transcription factors. NK cells represent a distinct group of innate lymphoid cells (ILCs) controlling viral infections and cancer.

In one aspect, the present invention provides at least one polynucleotide encoding a combination of at least three different transcription factors selected from the group consisting of: ETS1, TBET, NFIL3, EOMES, ID2, GATA3, ZFP105, IKZF3, ETS1, TOX, RUNX3, KLF12, ZEB2, IRF2, STAT5, IKZF1, ELF4, ZBTB16, GATA2 and ELF1.

As used herein “polynucleotide” refers to a nucleic acid molecule and includes genomic DNA, cDNA, RNA, mRNA, mixed polymers, recombinant nucleic acids, fragments and variants thereof, and the like.

As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

In another aspect, the present invention relates to at least one polynucleotide encoding a combination of at least three different transcription factors selected from the group consisting of: ETS1, TBET, NFIL3 and EOMES.

In one embodiment of the present invention, the combination of at least three transcription factors is a combination of the four transcription factors ETS1, TBET, NFIL3 and EOMES.

In one embodiment of the present invention, the combination further comprises one or more transcription factors selected from the group consisting of: ID2, GATA3, ZFP105, IKZF3, TOX, RUNX3, KLF12, ZEB2, IRF2, STAT5, IKZF1, ELF4, ZBTB16, GATA2 and ELF1.

In one embodiment, the combination of at least three transcription factors, of the at least one polynucleotide according to any one of the preceding claims, comprises an amino acid sequence selected from the group consisting of: SEQ. ID. NO: 1 to SEQ. ID. NO: 6. In another embodiment, the combination of at least three transcription factors, of the at least one polynucleotide according to any one of the preceding claims, are encoded by a polynucleotide sequence selected from the group consisting of: SEQ. ID. NO: 22 to SEQ. ID. NO: 27, or a sequence with at least 80%, such as at least 85%, such as at least 90%, such as at least 95% sequence identity thereof.

In one embodiment, the transcription factor ETS1, of the at least one polynucleotide according to any one of the preceding items, comprises the amino acid sequence SEQ. ID. NO: 1 or a biologically active variant thereof. In another embodiment, the transcription factor TBET comprises the amino acid sequence SEQ. ID. NO: 2, or a biologically active variant thereof. In one embodiment, the transcription factor NFIL3 comprises the amino acid sequence SEQ. ID. NO: 3, or a biologically active variant thereof. In one embodiment, the transcription factor EOMES comprises the amino acid sequence SEQ. ID. NO: 4, or a biologically active variant thereof. In one embodiment, the biologically active variant of EOMES comprises a sequence selected from the group consisting of: SEQ. ID. NOs: 5 to 6.

As used herein “biologically active variant” includes any derivative or variant of a molecule having substantially the same functional and/or biological properties of said molecule, such as binding properties, and/or the same structural basis, such as a peptidic backbone or a basic polymeric unit. It also refers to a molecule that exhibits the functional features as the transcription factors disclosed herein in a test, such as those disclosed in the present invention, where the test is conducted by a person skilled in the art. The test may identify transcription factors capable of activating the NCR1-tdTomato reporter.

In one embodiment, the polynucleotide encoding SEQ. ID. NO: 1 is SEQ. ID. NO: 22, or a sequence with at least 80%, such as at least 85%, such as at least 90%, such as at least 95% sequence identity thereof.

In one embodiment, the polynucleotide encoding SEQ. ID. NO: 2 is SEQ. ID. NO: 23, or a sequence with at least 80%, such as at least 85%, such as at least 90%, such as at least 95% sequence identity thereof.

In one embodiment, the polynucleotide encoding SEQ. ID. NO: 3 is SEQ. ID. NO: 24, or a sequence with at least 80%, such as at least 85%, such as at least 90%, such as at least 95% sequence identity thereof.

In one embodiment, the polynucleotide encoding SEQ. ID. NO:s 4 to 6 is selected from the group consisting of: SEQ. ID. NO: 25, SEQ. ID. NO: 26, and SEQ. ID. NO: 27, or a sequence with at least 80%, such as at least 85%, such as at least 90%, such as at least 95% sequence identity thereof.

In another embodiment, the combination of at least three transcription factors of the at least one polynucleotide as described herein, is: ETS1, TBET, NFIL3 and EOMES.

In another embodiment, the combination of at least three transcription factors of the at least one polynucleotide as described herein, is: ETS1, TBET and NFIL3.

In another embodiment, the combination of at least three transcription factors of the at least one polynucleotide as described herein, is: ETS1, TBET and EOMES.

In another embodiment, the combination of at least three transcription factors of the at least one polynucleotide as described herein, is: ETS1, NFIL3 and EOMES.

In another embodiment, the combination of at least three transcription factors of the at least one polynucleotide as described herein, is: TBET, NFIL3 and EOMES.

In one aspect, the present invention regards a construct or vector comprising the at least one polynucleotide as described herein.

As used herein the singular forms “a”, “an” and “the” include plural aspects unless the context clearly dictates otherwise. Thus, for example, reference to “a vector” includes a single vector, as well as two or more vector; reference to “a polynucleotide” includes one polynucleotide, as well as two or more polynucleotide; and so forth.

“Vector” as used herein means a nucleic acid sequence containing an origin of replication. A vector can be a vector, bacteriophage, bacteria, artificial chromosome or yeast artificial chromosome. A vector can be a DNA or RNA vector. A vector can be a self-replicating extrachromosomal vector, and a DNA plasmid.

In one embodiment, the vector is a viral vector. In one embodiment, the viral vector is selected from the group consisting of: lentiviral vector, retroviral vector, adenoviral vector, herpes viral vector, pox viral vector, flaviviral vector, rabdoviral vector, paramyxoviral vector, adeno-associated viral vector, reoviral vector, papillomaviral vector, picornaviral vector, caliciviral vector, hepadnaviral vector, togaviral vector, coronaviral vector, hepatitis viral vector, orthomyxoviral vector, bunyaviral vector and filoviral vector.

“Lentiviral vectors” or “member of the family of lentiviruses” as described herein are virus particles that contain a lentivirus-derived viral genome, lack the self-renewal ability, and have the ability to introduce a nucleic acid molecule into a host. Specifically, these vectors have a lentiviral backbone. The phrase “has a lentiviral backbone” means that the nucleic acid molecule included in the virus particles constituting the vectors is based on a lentiviral genome.

In one embodiment, the vector is an oncolytic vector. Oncolytic viruses are defined herein to generally refer to viruses that kill tumor or cancer cells more often than they kill normal cells.

In one embodiment, the construct or vector is synthetic mRNA, naked alphavirus RNA replicon or naked flavivirus RNA replicon.

In one embodiment, the construct or vector is a plasmid. The term “plasmid” is used herein interchangeably with the term “plasmid DNA” and encompasses the various plasmid forms i.e. open circular (oc), also known as nicked plasmid DNA and supercoiled (ccc) plasmid DNA.

In one embodiment, the vector further comprises a post-translational regulatory element (PRE) sequence. In one embodiment, the PRE sequence is selected from the group consisting of: a Woodchuck PRE (WPRE) or a hepatitis B virus (HBV) PRE (HPRE).

In one embodiment, the vector can further comprise other nucleic acid elements for the regulation, expression, stabilization of the construct or of other vector genetic elements, for example, promoters, enhancers, TATA-box, ribosome binding sites, IRES, as known to one of ordinary skill in the art.

In an embodiment the vectors can further comprise an internal ribosome entry site (IRES). An IRES can act as the sole ribosome binding site, or can serve as one of multiple ribosome binding sites of an mRNA. An mRNA containing more than one functional ribosome binding site can encode several peptides or polypeptides, such as the NK-inducing factors described herein, that are translated independently by the ribosomes (“multicistronic mRNA”). When nucleic acids are provided with an IRES, further optionally provided is a second translatable region. Examples of IRES sequences that can be used according to the disclosure include without limitation, those from picornaviruses (e.g. FMDV), pest viruses (CFFV), polioviruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SW) or cricket paralysis viruses (CrPV).

In one embodiment, the vector further comprises a promoter sequence controlling the transcription of the at least one polynucleotide as described herein. In one embodiment, the promoter sequence is selected from the group consisting of: Spleen Focus-Forming Virus (SFFV) promoter, cytomegalovirus (CMV) promoter, phosphoglycerate kinase (PGK) promoter, myelin basic protein (MBP) promoter, glial fibrillary acidic protein (GFAP) promoter, modified MoMuLV LTR containing myeloproliferative sarcoma virus enhancer (MNDU3), ubiquitin C promoter, EF-1 alpha promoter, or murine stem cell virus (MSCV) promoter.

In one embodiment, the viral vector comprises a tetracycline response element (TRE) and a minimal CMV promoter controlling the expression of TF coding region (pFUW-TetO). The vector may be used in combination with a vector containing the reverse tetracycline transactivator (rtTA) under the control of a constitutively active human ubiquitin C promoter (FUW-M2rtTA).

In one embodiment, the vector comprises a tetracycline response element (TRE) controlling the expression of TF coding regions in the same construct as the rtTA transactivator under the control of the constitutively active PGK promoter.

In one embodiment, the construct or vector is a CRISPR/CAS activation system. A person skilled in the art will understand that this system is able to activate endogenous transcription factors, enabling the use of a plasmid without transcription factors.

In one embodiment, the at least three transcription factors of the at least one polynucleotide is in the following sequential order from 5′ to 3′: ETS1, TBET, NFIL3 and EOMES.

In one embodiment, the at least three transcription factors of the at least one polynucleotide is in the following sequential order from 5′ to 3′: TBET, ETS1, NFIL3 and EOMES.

In one embodiment, the at least three transcription factors of the at least one polynucleotide is in the following sequential order from 5′ to 3′: NFIL3, ETS1, TBET and EOMES.

In one embodiment, the at least three transcription factors of the at least one polynucleotide is in the following sequential order from 5′ to 3′: ETS1, NFIL3, TBET and EOMES.

In one embodiment, the at least three transcription factors of the at least one polynucleotide is in the following sequential order from 5′ to 3′: TBET, NFIL3, ETS1 and EOMES.

In one embodiment, the at least three transcription factors of the at least one polynucleotide is in the following sequential order from 5′ to 3′: NFIL3, TBET, ETS1 and EOMES.

In one embodiment, the at least three transcription factors of the at least one polynucleotide is in the following sequential order from 5′ to 3′: NFIL3, TBET, EOMES and ETS1.

In one embodiment, the at least three transcription factors of the at least one polynucleotide is in the following sequential order from 5′ to 3′: TBET, NFIL3, EOMES and ETS1.

In one embodiment, the at least three transcription factors of the at least one polynucleotide is in the following sequential order from 5′ to 3′: EOMES, NFIL3, TBET and ETS1.

In one embodiment, the at least three transcription factors of the at least one polynucleotide is in the following sequential order from 5′ to 3′: NFIL3, EOMES, TBET and ETS1.

In one embodiment, the at least three transcription factors of the at least one polynucleotide is in the following sequential order from 5′ to 3′: TBET, EOMES, NFIL3 and ETS1.

In one embodiment, the at least three transcription factors of the at least one polynucleotide is in the following sequential order from 5′ to 3′: EOMES, TBET, NFIL3 and ETS1.

In one embodiment, the at least three transcription factors of the at least one polynucleotide is in the following sequential order from 5′ to 3′: EOMES, ETS1, NFIL3 and TBET.

In one embodiment, the at least three transcription factors of the at least one polynucleotide is in the following sequential order from 5′ to 3′: ETS1, EOMES, NFIL3 and TBET.

In one embodiment, the at least three transcription factors of the at least one polynucleotide is in the following sequential order from 5′ to 3′: NFIL3, EOMES, ETS1 and TBET.

In one embodiment, the at least three transcription factors of the at least one polynucleotide is in the following sequential order from 5′ to 3′: EOMES, NFIL3, ETS1 and TBET.

In one embodiment, the at least three transcription factors of the at least one polynucleotide is in the following sequential order from 5′ to 3′: ETS1, NFIL3, EOMES and TBET.

In one embodiment, the at least three transcription factors of the at least one polynucleotide is in the following sequential order from 5′ to 3′: NFIL3, ETS1, EOMES and TBET.

In one embodiment, the at least three transcription factors of the at least one polynucleotide is in the following sequential order from 5′ to 3′: TBET, ETS1, EOMES and NFIL3.

In one embodiment, the at least three transcription factors of the at least one polynucleotide is in the following sequential order from 5′ to 3′: ETS1, TBET, EOMES and NFIL3.

In one embodiment, the at least three transcription factors of the at least one polynucleotide is in the following sequential order from 5′ to 3′: EOMES, TBET, ETS1 and NFIL3.

In one embodiment, the at least three transcription factors of the at least one polynucleotide is in the following sequential order from 5′ to 3′: TBET, EOMES, ETS1 and NFIL3.

In one embodiment, the at least three transcription factors of the at least one polynucleotide is in the following sequential order from 5′ to 3′: ETS1, EOMES, TBET and NFIL3.

In one embodiment, the at least three transcription factors of the at least one polynucleotide is in the following sequential order from 5′ to 3′: EOMES, ETS1, TBET and NFIL3.

In one embodiment, the at least three transcription factors of the at least one polynucleotide is in the following sequential order from 5′ to 3′: ETS1, TBET and NFIL3.

In one embodiment, the at least three transcription factors of the at least one polynucleotide is in the following sequential order from 5′ to 3′: TBET, ETS1 and NFIL3.

In one embodiment, the at least three transcription factors of the at least one polynucleotide is in the following sequential order from 5′ to 3′: NFIL3, ETS1 and TBET.

In one embodiment, the at least three transcription factors of the at least one polynucleotide is in the following sequential order from 5′ to 3′: ETS1, NFIL3 and TBET.

In one embodiment, the at least three transcription factors of the at least one polynucleotide is in the following sequential order from 5′ to 3′: TBET, NFIL3 and ETS1.

In one embodiment, the at least three transcription factors of the at least one polynucleotide is in the following sequential order from 5′ to 3′: NFIL3, TBET and ETS1.

In one embodiment, the at least three transcription factors of the at least one polynucleotide is in the following sequential order from 5′ to 3′: ETS1, TBET and EOMES.

In one embodiment, the at least three transcription factors of the at least one polynucleotide is in the following sequential order from 5′ to 3′: TBET, ETS1 and EOMES.

In one embodiment, the at least three transcription factors of the at least one polynucleotide is in the following sequential order from 5′ to 3′: EOMES, ETS1 and TBET.

In one embodiment, the at least three transcription factors of the at least one polynucleotide is in the following sequential order from 5′ to 3′: ETS1, EOMES and TBET.

In one embodiment, the at least three transcription factors of the at least one polynucleotide is in the following sequential order from 5′ to 3′: TBET, EOMES and ETS1.

In one embodiment, the at least three transcription factors of the at least one polynucleotide is in the following sequential order from 5′ to 3′: EOMES, TBET and ETS1.

In one embodiment, the at least three transcription factors of the at least one polynucleotide is in the following sequential order from 5′ to 3′: ETS1, NFIL3 and EOMES.

In one embodiment, the at least three transcription factors of the at least one polynucleotide is in the following sequential order from 5′ to 3′: NFIL3, ETS1 and EOMES.

In one embodiment, the at least three transcription factors of the at least one polynucleotide is in the following sequential order from 5′ to 3′: EOMES, ETS1 and NFIL3.

In one embodiment, the at least three transcription factors of the at least one polynucleotide is in the following sequential order from 5′ to 3′: ETS1, EOMES and NFIL3.

In one embodiment, the at least three transcription factors of the at least one polynucleotide is in the following sequential order from 5′ to 3′: NFIL3, EOMES and ETS1.

In one embodiment, the at least three transcription factors of the at least one polynucleotide is in the following sequential order from 5′ to 3′: EOMES, NFIL3 and ETS1.

In one embodiment, the at least three transcription factors of the at least one polynucleotide is in the following sequential order from 5′ to 3′: TBET, NFIL3 and EOMES.

In one embodiment, the at least three transcription factors of the at least one polynucleotide is in the following sequential order from 5′ to 3′: NFIL3, TBET and EOMES.

In one embodiment, the at least three transcription factors of the at least one polynucleotide is in the following sequential order from 5′ to 3′: EOMES, TBET and NFIL3.

In one embodiment, the at least three transcription factors of the at least one polynucleotide is in the following sequential order from 5′ to 3′: TBET, EOMES and NFIL3.

In one embodiment, the at least three transcription factors of the at least one polynucleotide is in the following sequential order from 5′ to 3′: NFIL3, EOMES and TBET.

In one embodiment, the at least three transcription factors of the at least one polynucleotide is in the following sequential order from 5′ to 3′: EOMES, NFIL3 and TBET.

In one aspect, the present invention provides a cell comprising the at least one polynucleotide, and/or the construct or vector as described herein.

In one aspect, the present invention relates to a pharmaceutical composition comprising the at least one polynucleotide, the construct or vector and/or the cell as described herein. In one embodiment, the pharmaceutical composition further comprises a suitable carrier. These compositions may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, plasticizers, crystallization inhibitors, wetting agents, bulk filling agents, solubilizers, bioavailability enhancers, solvents, pH-adjusting agents and combinations thereof.

In one aspect, the present invention provides a method for reprogramming or inducing any cell into a natural killer cell or progenitor, the method comprising the following steps:
a. transducing a cell with the at least one polynucleotide, or the construct or vector as described herein;
b. culturing and expanding the transduced cell in a cell media; and
c. obtaining a reprogrammed cell.

In one embodiment, the induced or reprogrammed progenitor is CD34, CD38 and/or CD10 positive. A person skilled in the art will appreciate that these are immature cell markers. The reprogrammed cells of the present invention may transition from progenitors to mature cells in culture. A person skilled in the art will appreciate that this makes them easier to expand in culture. The mature cell state may be characterised by the lack of expression of CD34 and CD10, and an expression of CD8, CD38 and DX5 (encoded by the ITGA2 gene).

As used herein, “+”, “⁺” and “positive” are used interchangeably.

In one embodiment, the method further comprises culturing the transduced cell in a media comprising one or more cytokine(s). In another embodiment, the one or more cytokine(s) are selected from the group consisting of: SCF, FLT3L, IL-3, IL-7 and IL-15

In one embodiment of the present invention, the cell is a mammalian cell. In one embodiment, the cell is a human cell. In one embodiment, the cell is selected from the group consisting of: a stem cell, a differentiated cell, a cancer cell or a tumor cell.

In one embodiment, the stem cell is selected from the group consisting of: a mesenchymal stem cell, a pluripotent stem cell and a hematopoietic stem cell.

In some embodiments of the present invention, the somatic cell is a fibroblast cell.

Essentially any primary somatic cell type can be used for producing induced NK cells or reprogramming somatic cells to induced NK cells according to the presently described compositions, constructs, vectors, cells and methods. Some non-limiting examples of primary somatic cells useful in the various aspects and embodiments of the methods described herein include, but are not limited to: fibroblasts, epithelial cells, endothelial cells, neuronal cells, adipose cells, cardiac cells, skeletal muscle cells, hematopoietic or immune cells, hepatic cells, splenic cells, lung cells, circulating blood cells, gastrointestinal cells, renal cells, bone marrow cells, and pancreatic cells, as well as stem cells from which those cells are derived. The cell can be a primary cell isolated from any somatic tissue including, but not limited to: spleen, bone marrow, blood, brain, liver, lung, gut, stomach, intestine, fat, muscle, uterus, skin, spleen, endocrine organ and bone. The term “somatic cell” further encompasses, in some embodiments, primary cells grown in culture, provided that the somatic cells are not immortalized. Where the cell is maintained under in vitro conditions, conventional tissue culture conditions and methods can be used, and are known to those of skill in the art. Isolation and culture methods for various primary somatic cells are well within the abilities of one skilled in the art.

In some embodiments, the somatic cell can be a hematopoietic lineage cell.

In some embodiments of the present invention, a somatic cell to be reprogrammed or made into an induced NK cell is a cell of hematopoietic origin. As used herein, the terms “hematopoietic-derived cell,” “hematopoietic-derived differentiated cell,” “hematopoietic lineage cell,” and “cell of hematopoietic origin” refer to cells derived or differentiated from a multipotent hematopoietic stem cell (HSC). Accordingly, hematopoietic lineage cells for use with the compositions, constructs, vectors, cells and methods described herein include multipotent, oligopotent, and lineage-restricted hematopoietic progenitor cells, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, and lymphocytes (e.g., T-lymphocytes, which carry T-cell receptors (TCRs), B-lymphocytes or B cells, which express immunoglobulin and produce antibodies, NK cells, NKT cells, and innate lymphocytes). As used herein, the term “hematopoietic progenitor cells” refer to multipotent, oligopotent, and lineage-restricted hematopoietic cells capable of differentiating into two or more cell types of the hematopoietic system, including, but not limited to, granulocytes, monocytes, erythrocytes, megakaryocytes, and lymphocytes B-cells and T-cells. Hematopoietic progenitor cells encompass multi-potent progenitor cells (MPPs), common myeloid progenitor cells (CMPs), common lymphoid progenitor cells (CLPs), granulocyte-monocyte progenitor cells (GMPs), and pre-megakaryocyte-erythrocyte progenitor cell. Lineage-restricted hematopoietic progenitor cells include megakaryocyte-erythrocyte progenitor cells (MEP), ProB cells, PreB cells, PreProB cells, ProT cells, double-negative T cells, pro-NK cells, pre-granulocyte/macrophage cells, granulocyte/macrophage progenitor (GMP) cells, and pro-mast cells (ProMCs).

Cells of hematopoietic origin for use in the compositions, constructs, vectors, cells and methods described herein can be obtained from any source known to comprise these cells, such as fetal tissues, umbilical cord blood, bone marrow, peripheral blood, mobilized peripheral blood, spleen, liver, thymus, lymph, etc. Cells obtained from these sources can be expanded ex vivo using any method acceptable to those skilled in the art prior to use in with the compositions, constructs, vectors, cells and methods for making the induced NK cells described herein. For example, cells can be sorted, fractionated, treated to remove specific cell types, or otherwise manipulated to obtain a population of cells for use in the methods described herein using any procedure acceptable to those skilled in the art.

In one embodiment of the present invention, the transduced cell is cultured during at least 2 days, such as at least 5 days, such as at least 8 days, such as at least 10 days, such as at least 12 days.

In one embodiment of the present invention, the reprogrammed cell is NCR1 positive.

In one embodiment of the present invention, the reprogrammed cell is CD34 positive.

In one embodiment of the present invention, the reprogrammed cell is CD56 positive.

In one aspect, the present invention provides an induced natural killer cell or progenitor obtained by the method as described herein.

In one embodiment of the present invention, the cell, the reprogrammed and/or induced cell is NCR1 positive. In another embodiment of the present invention, the cell, the reprogrammed and/or induced cell is CD34 positive. In another embodiment of the present invention, the cell, the reprogrammed and/or induced cell is CD56 positive.

In one embodiment, the present invention relates to the at least one polynucleotide, the construct or vector, the cell, and/or the induced natural killer cell as described herein, for use in medicine.

In one embodiment, the present invention relates to the at least one polynucleotide, the construct or vector, the cell, and/or the induced natural killer cell as described herein, for use in the treatment of cancer.

In one embodiment, the present invention relates to the at least one polynucleotide, the construct or vector, the cell, and/or the induced natural killer cell as described herein, for use in immunotherapy.

### EXAMPLES

### Example 1. Identifying Candidate Transcription Factors for NK Cell Reprogramming

Method

To identify candidate transcription factors (TF) to induce NK cell identity and their function as cytotoxic innate lymphocytes, we used three complementary approaches: (i) a developed predictive computational tool, GPSMatch; (ii) literature mining; and (iii) analysis of available gene expression datasets (GeneAtlas MOE430).

Results

We identified 19 NK-inducing candidate TFs that are enriched in NK cells when compared with other tissues and cell types, including T and B lymphocytes (FIG. 2).

Conclusions

19 candidate TFs were identified to instruct NK cell identity in other cell types.

### Example 2. Screening NK-Inducing Transcription Factors

Method

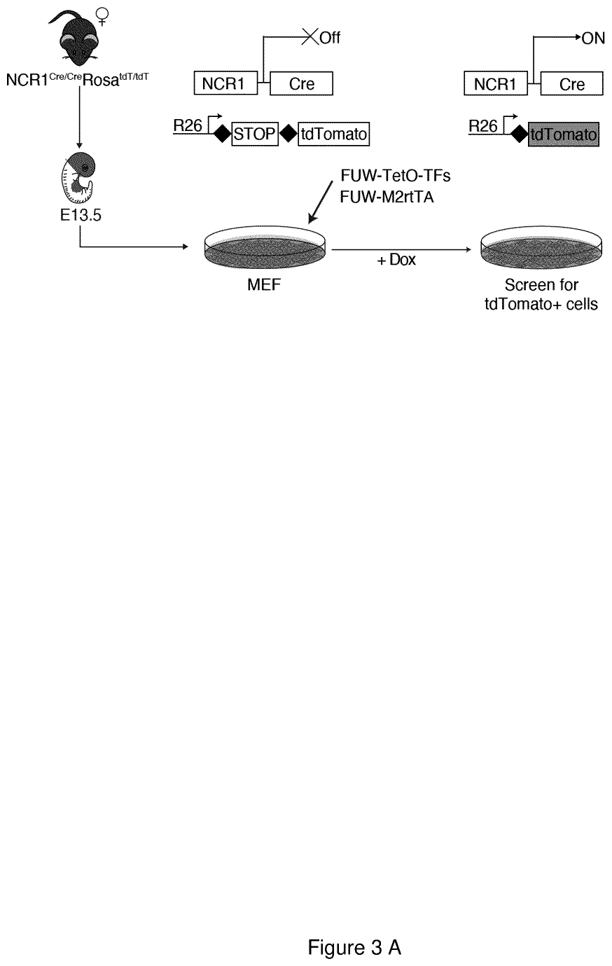

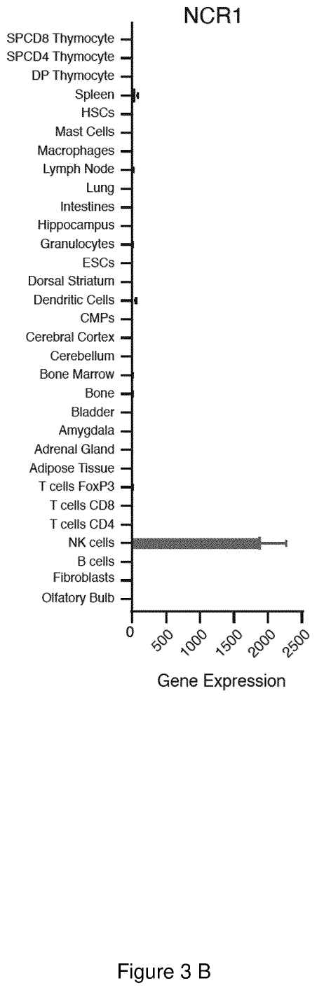

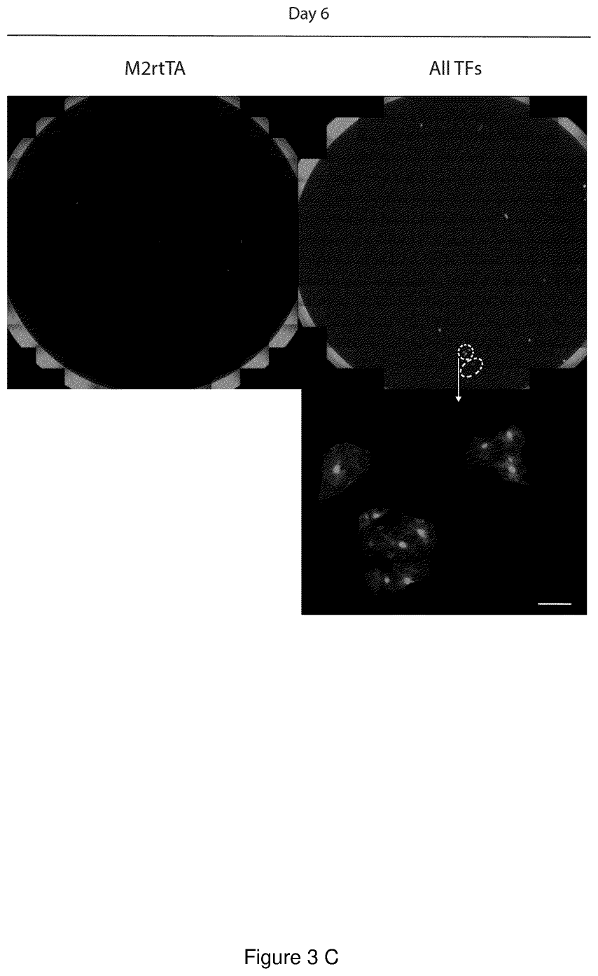

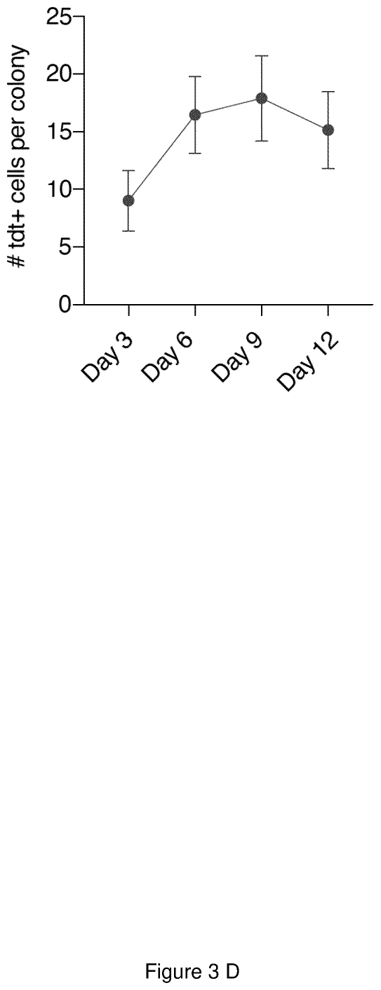

To screen for NK-inducing factors, 19 candidate TFs were expressed in primary cultures of mouse embryonic fibroblasts (MEFs) harboring a NK-specific reporter system (Ncr1-Cre X R26-stop-tdT mouse, hereafter called NCR1-tdTomato) (FIG. 3A). NCR1 is expressed specifically in NK cell lineages (FIG. 3B).

Isolation of MEFs

Ncr1Cre/Cre (Eckelhart el al., 2011) animals were crossed with Rosa26-stopflox-tdTomato reporter mice to generate double homozygous Ncr1Cre/Cre RosatdTomato/tdTomato (NCR1-tdTomato) mice. All animals were housed under controlled temperature (23±2° C.), subject to a fixed 12-h light/dark cycle, with free access to food and water.

Primary cultures of MEFs were isolated from E13.5 embryos of Ncr1-cre mice. Head, fetal liver and all internal organs were removed, and the remaining tissue was mechanically dissociated. Dissected tissue was enzymatic digested using 0.12% trypsin/0.1 mM Ethylenediaminetetraacetic acid (EDTA) solution (3 mL per embryo), and incubation at 37° C. for 15 min. Additional 3 mL of same solution per embryo were added, followed by another 15 min incubation period. A single cell suspension was obtained and plated in 0.1% gelatin-coated 10-cm tissue culture dishes in growth media. Cells were grown for 2-3 days until confluence, dissociated with Tryple Express and frozen in Fetal Bovine Serum (FBS) 10% dimethyl sulfoxide (DMSO). Before plating for lentiviral transduction, MEFs were sorted to remove residual CD45⁺ and tdTomato⁺ cells that could represent cells with hematopoietic potential. MEFs used for screening and in the following experiments were tdTomato⁻ CD45⁻ with purity above 99% and expanded up to 4 passages.

Transduction

Before plating for lentiviral transduction, MEFs were sorted to remove residual CD45+ and tdTomato positive cells that could represent cells with hematopoietic potential. NCR1-tdTomato MEFs were maintained in growth medium [Dulbecco's modified eagle medium (DMEM) supplemented with 10% (v/v) FBS, 2 mM L-Glutamine and antibiotics (10 μg/ml Penicillin and Streptomycin)]. All cells were maintained at 37° C. and 5% (v/v) CO2. NCR1-tdTomato MEFs were seeded at a density of 40,000 cells per well on 0.1% gelatin coated 6-well plates. Cells were incubated overnight with a ratio of 1:1 FUW-TetO-TFs and FUW-M2rtTA lentiviral particles in growth media supplemented with 8 μg/mL polybrene. Cells were transduced twice in consecutive days and after overnight incubation, media was replaced with fresh growth media. After the second transduction, growth media was supplemented with Doxycycline (1 μg/mL)—day 0. Media was changed every 2-3 days for the duration of the cultures. Emerging tdTomato positive cells were analyzed 3-12 days post-transduction by microscopy. The entire well of a 6-well plate was acquired in an automated Zeiss CD7. For Flow Cytometry analysis, transduced NCR1-tdTomato MEFs were dissociated with TrypLE Express, resuspended in PBS 5% FBS and kept at 4° C. prior analysis in BD LSRFortessa (BD Biosciences).

Results

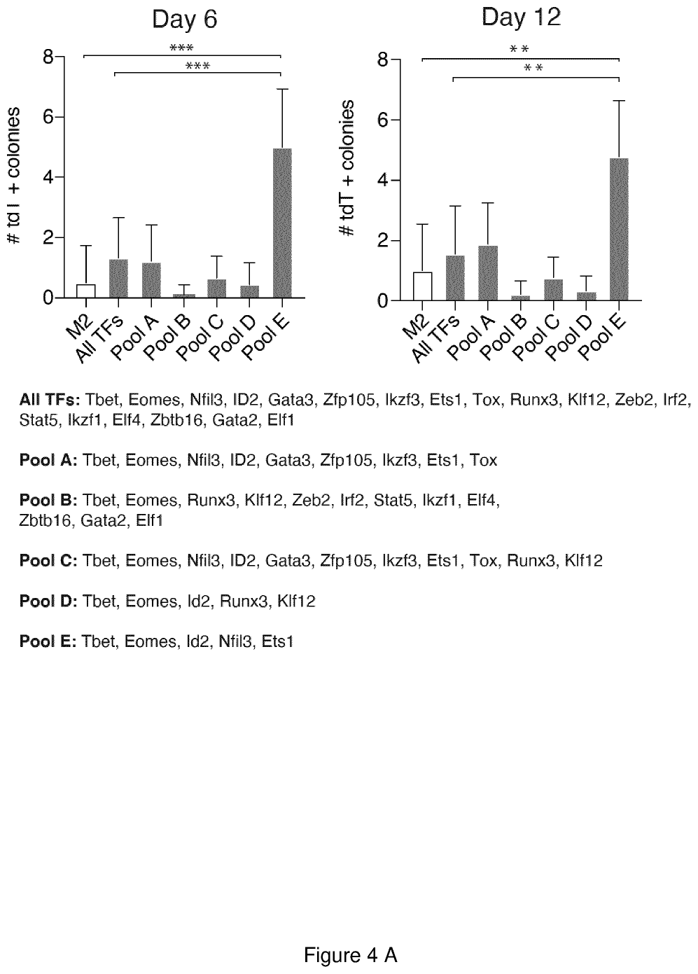

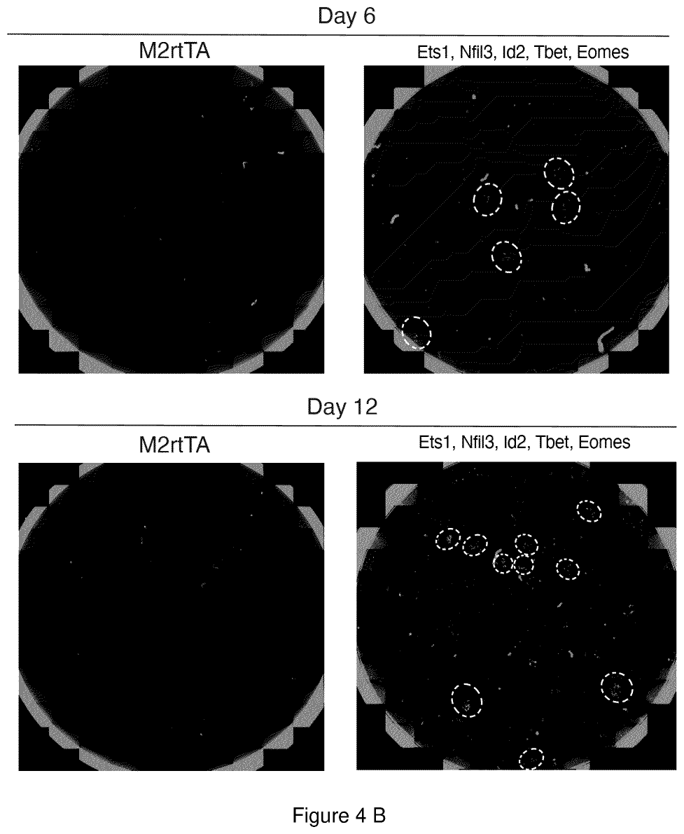

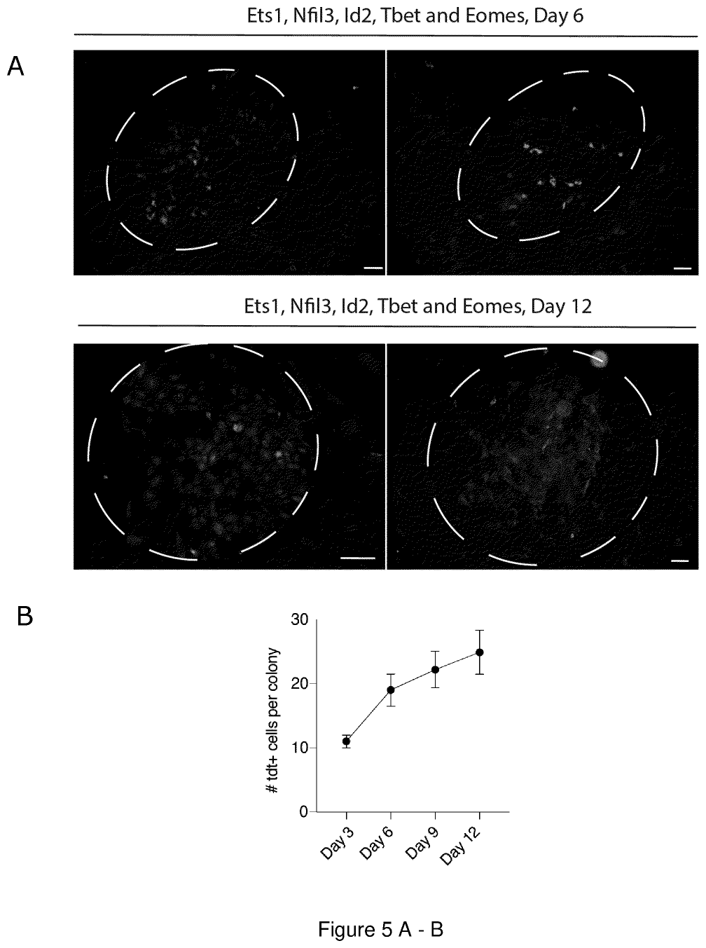

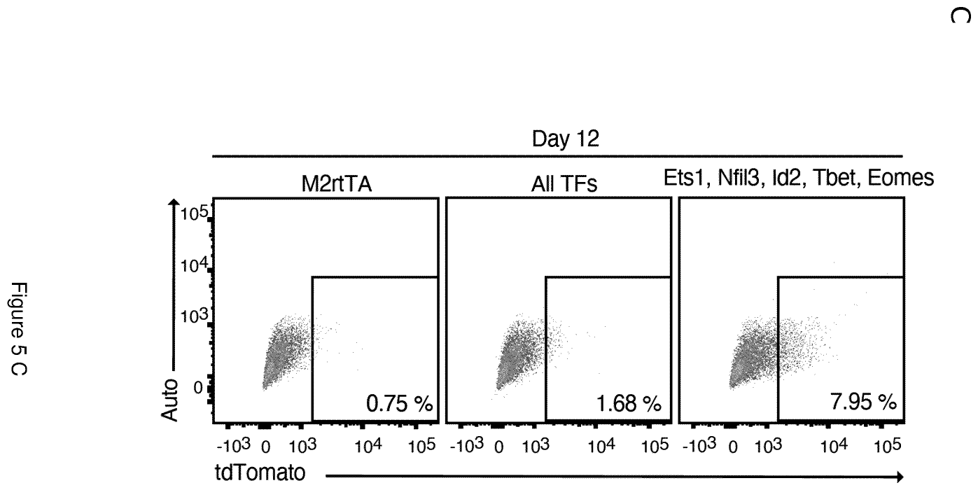

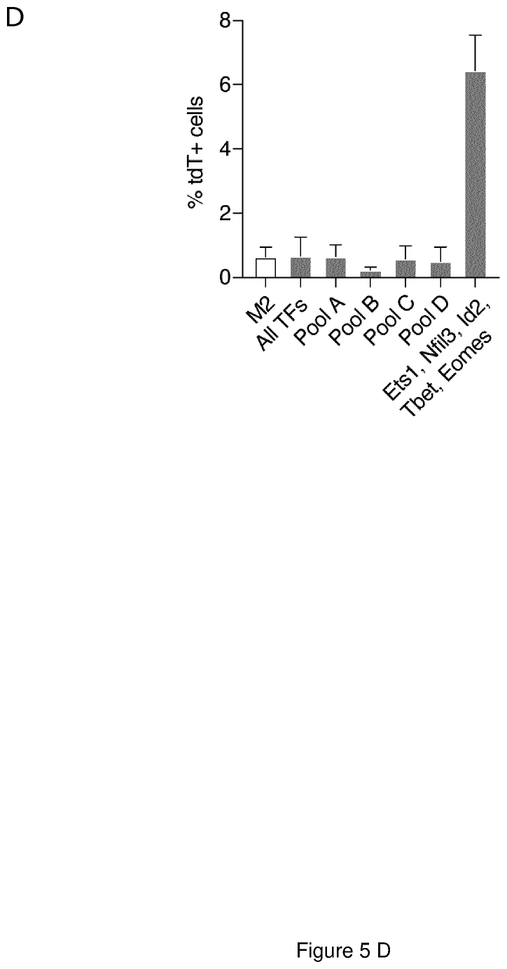

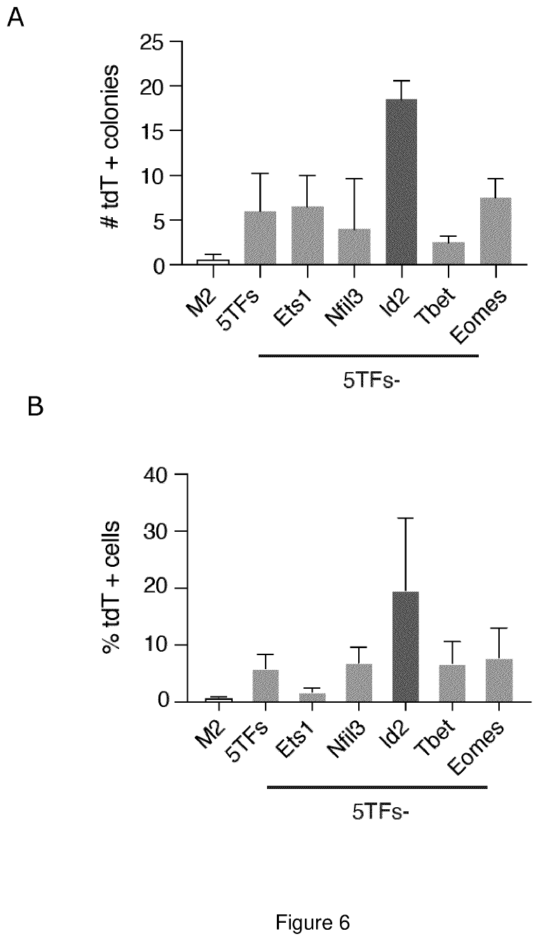

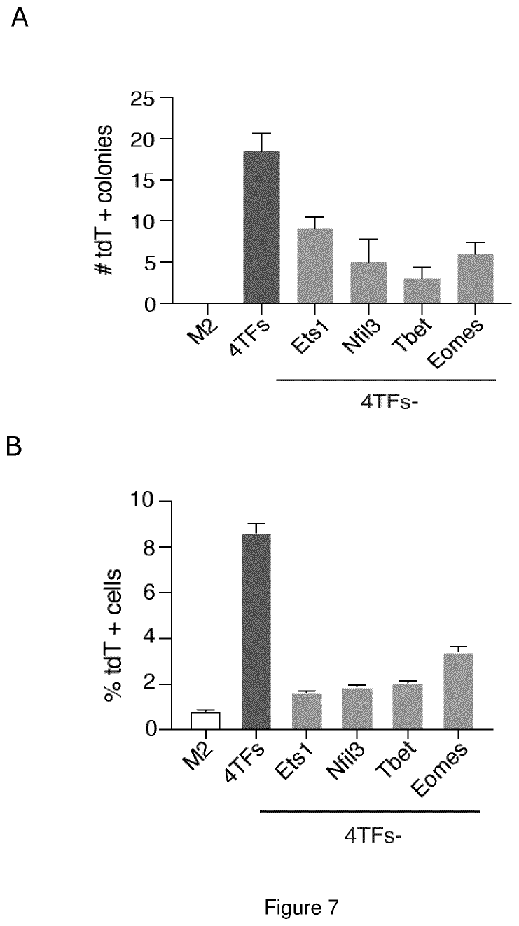

Expression of all 19 candidate TFs induce tdTomato-positive (tdT+) colonies that emerge asynchronously between day 3 and day 6, and expand in culture (FIGS. 3 C and D). The number of tdT+ colonies increased after overexpression of smaller pools of transcription factors, especially with a combination of 5 candidate TFs composed by ETS1, Nfi3, ID2, EOMES and TBET (FIG. 4). tdT+ colonies induced by the 5 TFs pool continue expanding in culture between day 3 and day 12 of reprogramming (FIG. 5). Additionally, this TF pool induces 6% of tdT+ cells, suggesting that the minimal combination required to induce NCR1-tdT activation is contained within this pool (Figure C and D). To define the minimal combination, we removed one TF individually from the TF pool and showed that ID2 was dispensable for the reprogramming process. The discovered combination of 4 TFs increase the number of tdT+ colonies (FIG. 6A) and induces ˜20% tdT+ cells at day 12 of reprogramming (FIG. 6 B). Another round of TF exclusion was conducted, which showed that a combination of three TFs are sufficient to increase the number of tdT+ colonies and percentage of tdT+ cells compared to control.

Conclusions

Screening with NCR1-tdTomato reporter allows identification of NK-TF instructors. These results indicated that a combination of three TFs selected from ETS1, NFIL3, EOMES and TBET are required and sufficient to activate the NCR1-reporter and induce expandable NCR1-positive colonies. A combination of four TFs (ETS1, NFIL3, TBET and EOMES) yielded the largest increase of the NCR1-reporter. NK reprogramming is fast (starting at day 3) and efficient (˜20% by day 12).

### Example 3. ETS1, NFIL3, TBET and EOMES are Enriched in NK Cells

Method

Gene expression data (GeneAtlas MOE430) was log-transformed and normalized to a 0-1 range for each gene followed by a search for the highest average expression for ETS1+NFIL3+TBET⁺EOMES.

Results

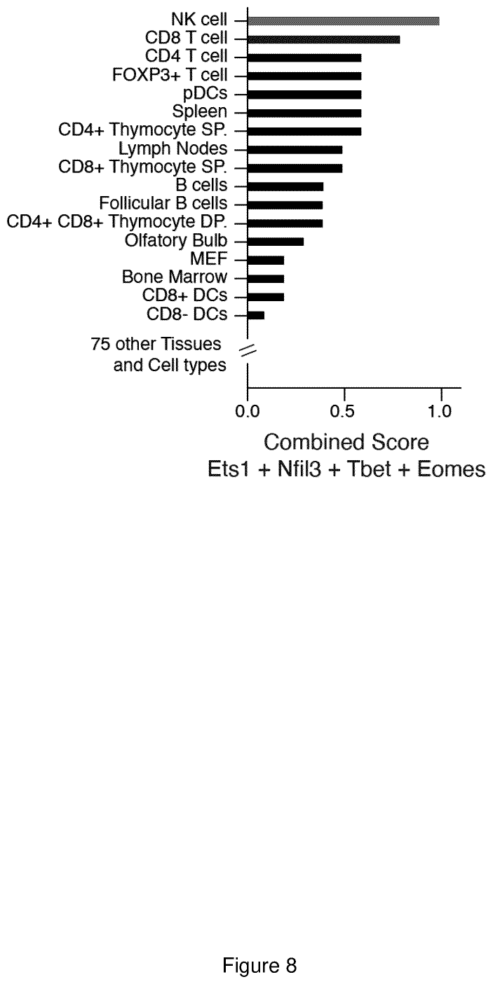

Combined expression of ETS1, NFIL3, TBET and EOMES is highly associated with NK cells (FIG. 8).

Conclusions

These results validate the identification of ETS1, NFIL3, TBET and EOMES as the combination to induce NK cell identity.

### Example 4. RORα, SMAD7, FOS, JUN and NFATC2 Combination does not Activate NCR1-tdTomato Reporter

Method

Screening with the NCR1-tdTomato reporter was conducted as described in Example 2, and the gene expression data was analyzed as described in Example 3.

Results

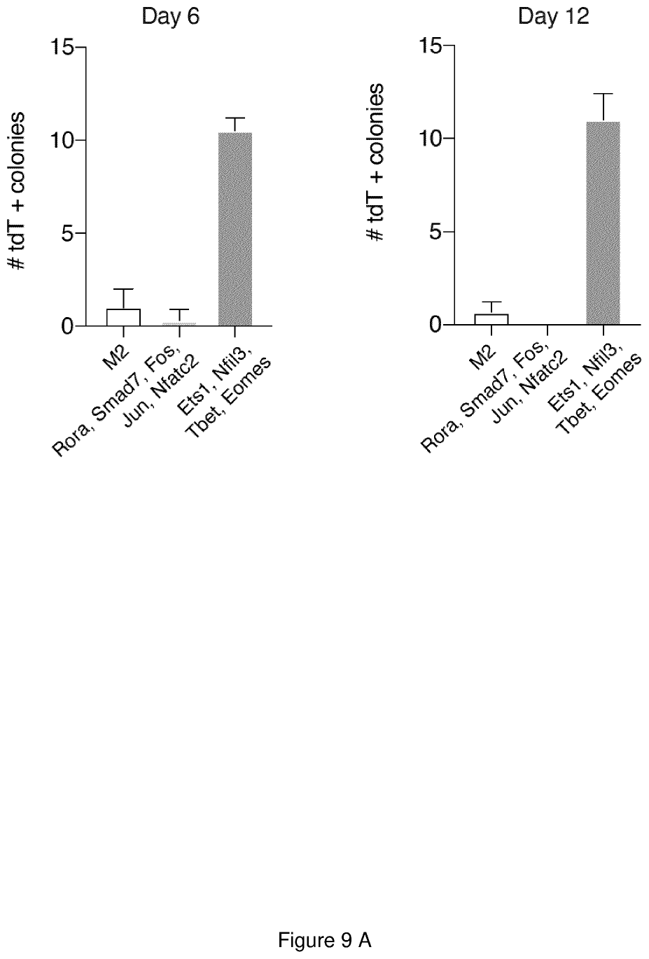

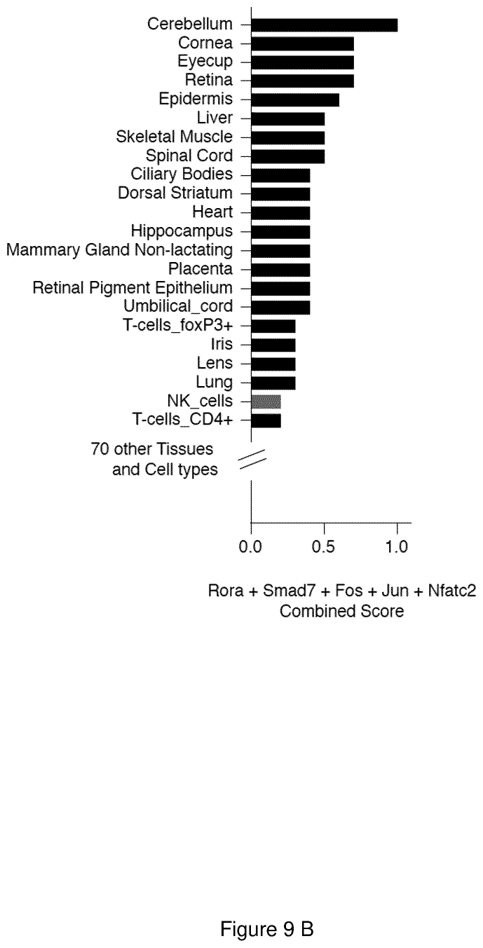

Expression of RORα, SMAD7, FOS, JUN and NFATC2 does not activate NCR1 reporter at day 6 or day 12 of reprogramming (FIG. 9A). Furthermore, gene expression analysis shows that combined expression of RORα+SMAD7+FOS+JUN+NFATC2 is not enriched in NK cells among other tissues and cell types (FIG. 9 B).

Conclusions

These results show that RORα, SMAD7, FOS, JUN and NFATC2 are not enriched in NK cells and do not activate the NCR1 reporter.

### Example 5. ETS1, NFIL3, TBET and EOMES Induce Global Transcriptional Changes to the NK Cell Lineage

Method

Untransduced MEFs and MEFs transduced with ETS1, NFIL3, TBET and EOMES were dissociated using TrypLE Express, re-suspended in PBS with 5% and FACS sorted at day 3 (tdT+), day 6 (tdT+), and day 12 (tdT+). Purified MEFs (5000 cells), tdT+d3 (5000 cells), tdT+d6 (5000 cells), tdT+d12 (5000 cells) were loaded on a 10× Chromium (10× Genomics) according to manufacturer's protocol. Single-cell RNA-Seq libraries were prepared using Chromium Single Cell 3′ v3.1 Reagent Kit (10× Genomics) according to manufacturer's protocol. Single cells were isolated into droplets together with gel beads coated with unique primers bearing 10× unique molecular identifiers (UMI) and poly(dT) sequences. Reverse transcription reactions were performed to generate barcoded full-length cDNA. Indexed sequencing libraries were constructed using the reagents from the Chromium Single Cell 3′ v3.1 Reagent Kit (10× Genomics). Library quantification and quality assessment was determined using Qubit and Agilant TapeStation. Indexed libraries were sequenced on an Illumina NovaSeq 6000 S2 100 FlowCell v1. Coverage of approximately 100,000 reads per single cell was obtained for tdT+ day 3, day 6, and day 12 samples and 25,000 reads per single cell for MEFS sample.

Results

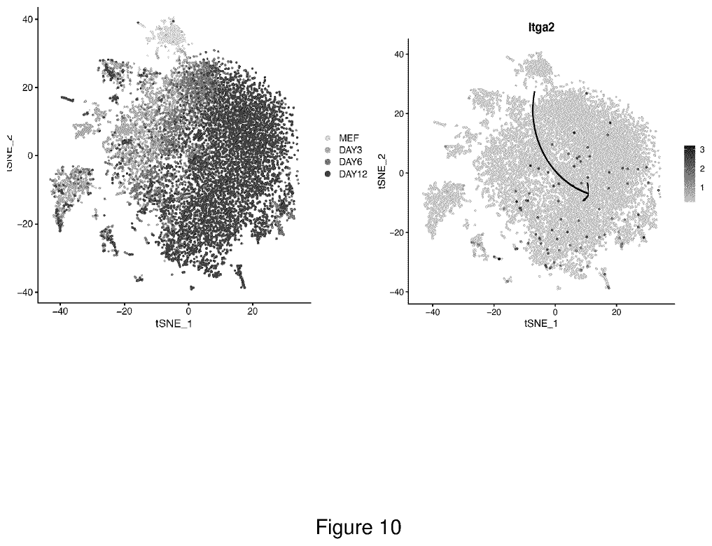

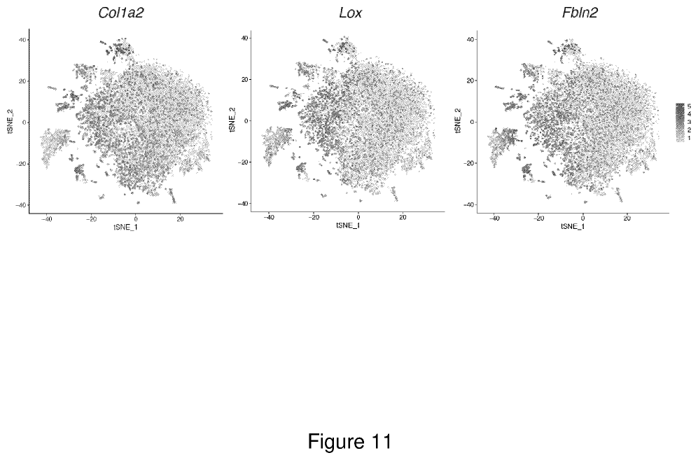

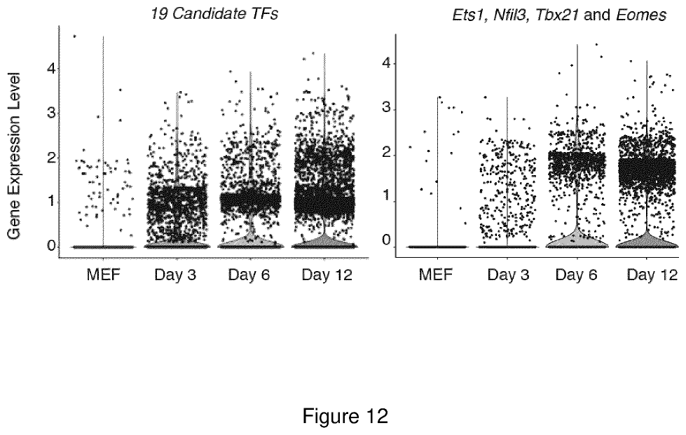

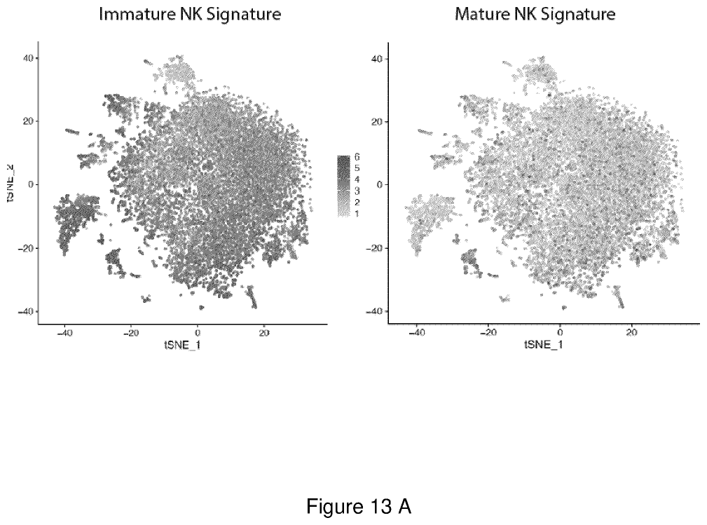

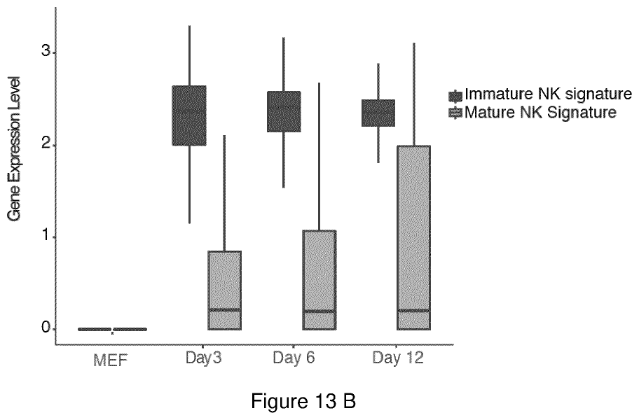

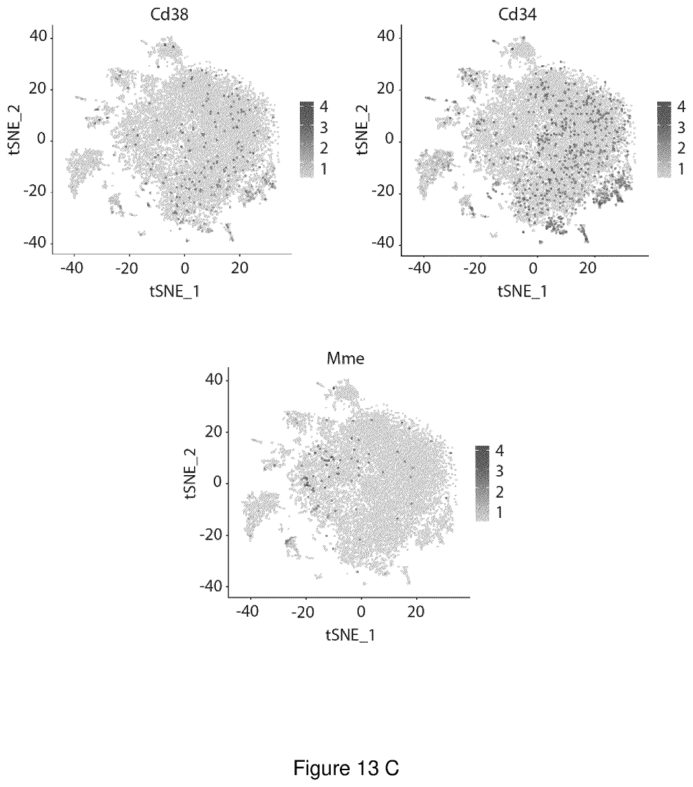

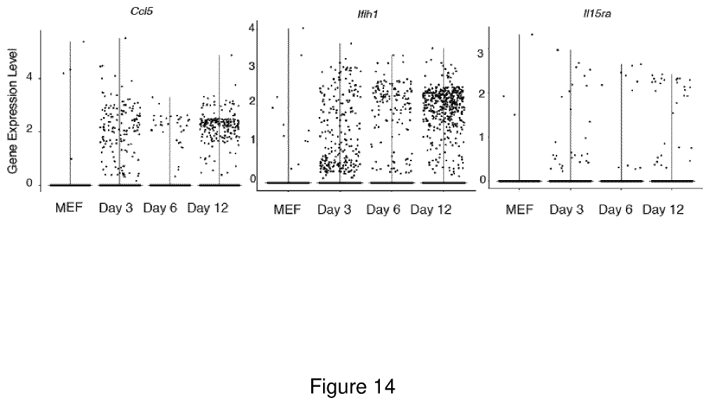

To determine whether the 4 TFs were inducing a NK cell identity in the MEFS, tdT+ cells at day 3, 6 and 12 of reprogramming were profiled by single cell RNA-sequencing (scRNA-seq) with the 10× genomics platform. TdT+ cells showed global transcriptional changes starting as early as day 3 and progressively at day 6 and 12 when fibroblast-specific genes were silenced (FIGS. 10A and 11). At day 12 mature NK genes such as Itga2 were also detected (FIG. 10 B). Combined expression of the 4 TFs imposed a NK-like transcriptional network, inducing the expression of NK-specific 19 candidate TFs at day 3 and progressively increasing until day 12 of reprogramming (FIG. 12). Endogenous expression of Ets1, Nfil3, Tbx21 (encoding Tbet) and Eomes was also activated during reprogramming. Additionally, scRNA-seq analysis revealed that the 4 TFs elicit global transcriptome reprogramming towards NK cell progenitor identity, inducing upregulation of NK progenitor genes including CD38, CD34 and MME (encoding for CD10) (FIG. 13 C). Induced NK cells generated with 4 TFs were highly enriched in an immature NK cell signature (Bezman et al. 2012) from the early stages of reprogramming. Accordingly, mature NK gene expression signature progressively increases at later reprogramming stages (FIGS. 13 A and B). Importantly, we detected an increased expression of genes associated with NK function including ccl5, Ifih1 and H15ra (FIG. 14).

Conclusions

These data demonstrated that ETS1, NFIL3, TBET and EOMES instruct the reprogramming of fibroblasts towards the NK cell lineage and impose a progenitor NK identity.

### Example 6. Enforced Expression of ETS1, NFIL3, TBET and EOMES Generate Human CD34 and CD56 Positive Cells

Method

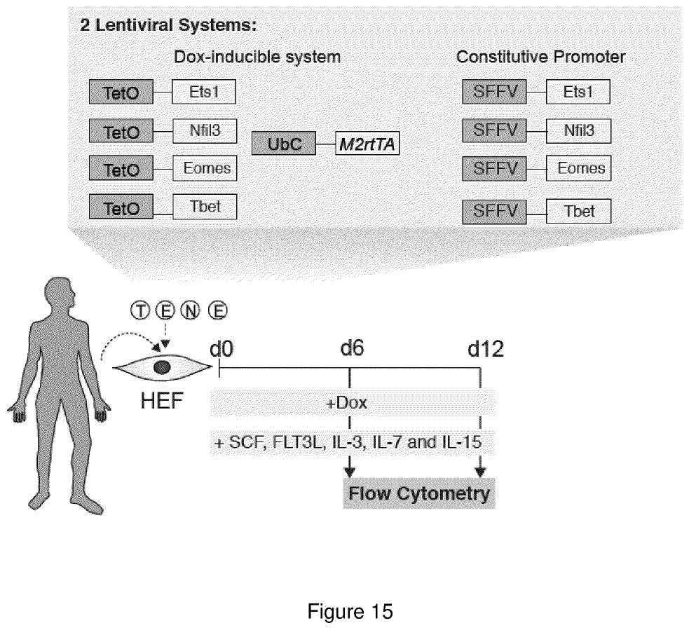

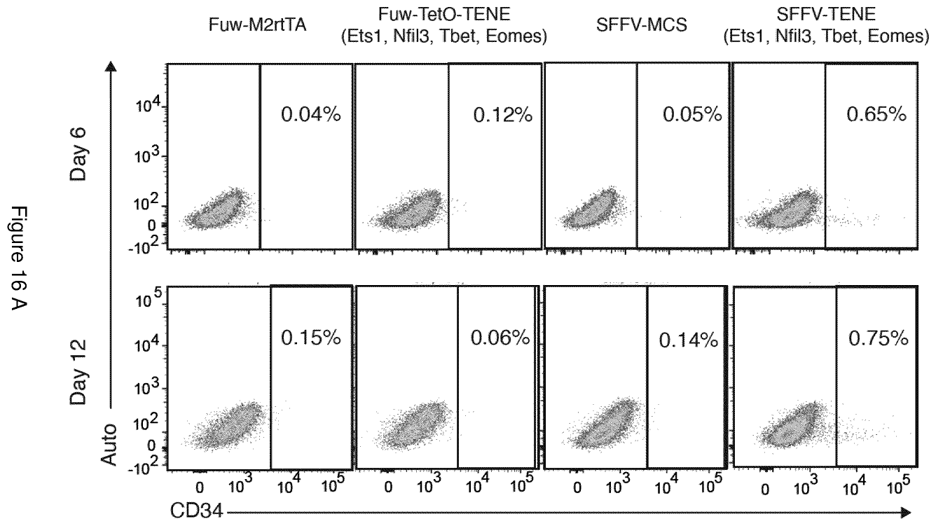

Human Embryonic Fibroblasts (HEFs) were seeded at a density of 30,000 cells per well on 0.1% gelatin coated 6-well plates. Cells were incubated overnight with a ratio of 1:1 FUW-TetO-TFs and FUW-M2rtTA lentiviral particles. Alternatively, cells were incubated with SFFV-MCS or SFFV-TFs lentiviral particles at the same multiplicity of infection (MOI). Lentiviral particles were added to growth media supplemented with 8 μg/mL polybrene. After overnight incubation, media was replaced with fresh growth media. For FUW-TetO transduced cells, growth media was supplemented with Doxycycline (1 μg/mL). Media was changed every 2-3 days for the duration of the cultures. Cytokine cocktail including stem cell factor (SCF) (10 ug/mL), FMS-like tyrosine kinase 3 ligand (FLT3L) (10 ug/mL), Interleukin-3 (IL-3) (5 ug/mL), IL-7 (5 ug/mL) and IL-15-(10 ug/mL) was added to culture when indicated. NK reprogramming was assessed 6 and 12 days after transduction by flow cytometry (FIG. 15). For flow cytometry, transduced HEFs were dissociated with TrypLE Express, resuspended in PBS 5% FBS and stained with mouse anti-human CD34 antibody or anti-human CD56. Mouse serum at 1% was used to prevent unspecific antibody binding. Cells were kept at 4° C. prior analysis in BD LSRFortessa (BD Biosciences).

Results

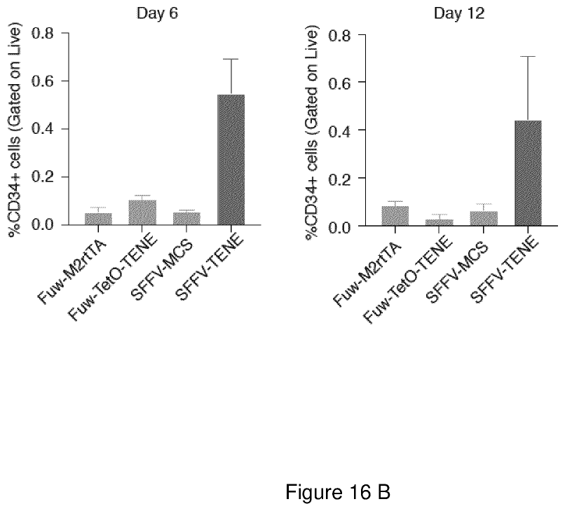

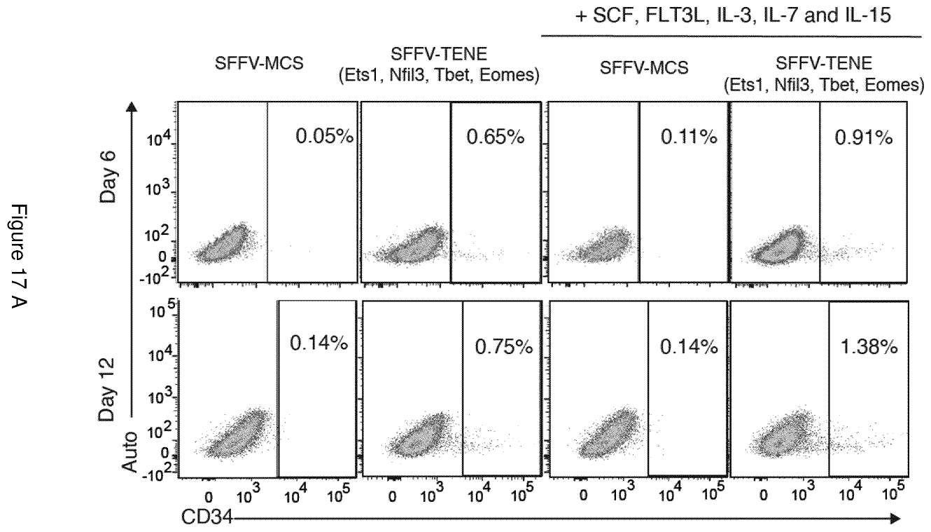

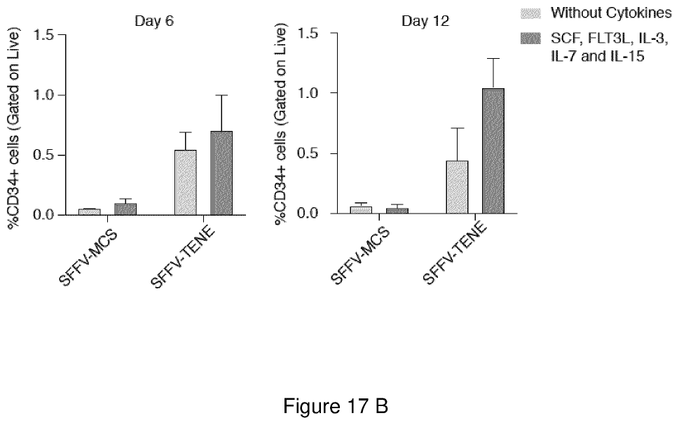

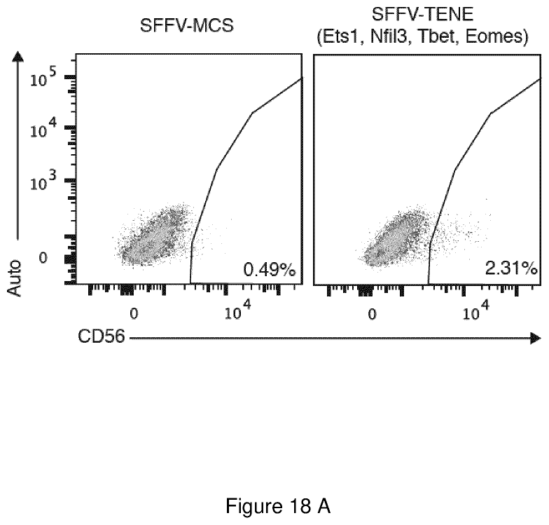

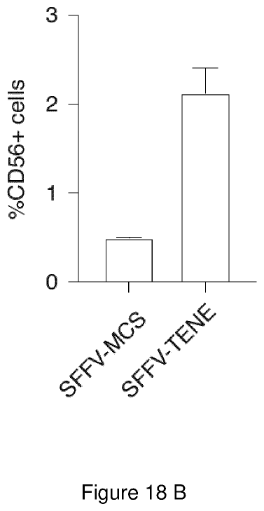

Combined expression of ETS1, NFIL3, TBET and Eomes cloned individually into pSFFV induced a rare population of human CD34 positive (CD34+) cells at day 6 and 12 of reprogramming (FIG. 16). CD34+ cells were not observed in empty vector (MCS) control. Culture in the presence of a NK-differentiation and activation cytokine cocktail increased reprogramming efficiency 1.5-fold at day 6 and approximately 2-fold at day 12 of reprogramming (FIG. 17). Additionally, combined expression of the 4 TFs induced human CD56 positive (CD56+) cells, 6 days after transduction (FIG. 18)

Conclusions

These results suggest that 4 TFs-ETS1, NFIL3, TBET and EOMES are sufficient to induce a combination of human CD34+NK progenitor identity and CD56+ mature NK cells. Additionally, these data indicates that constitutive lentiviral vector SFFV enables reprogramming from human cells at higher efficiencies than inducible FUW-TetO system. Addition of SCF, FLT3L, IL-3, IL-7 and IL-15 improve NK reprogramming process.

### Example 7. NK-Based Immunotherapies can Target Liquid and Solid Cancers

The cells produced by the methods described herein can be used to treat or alleviate several cancers and tumors including hematologic cancers such as leukemia, lymphoma as in NCT01807611, chronic myeloid leukemia, multiple myeloma (NCT00720785, NCT02955550), non-Hodgkin's lymphoma, diffuse Large B-Cell Lymphoma, Follicular Lymphoma, Mantle Cell Lymphoma, B-Lymphoid Malignancies, Acute Lymphocytic Leukemia (NCT03019640, NCT03579927, NCT03056339), Acute Myeloid Leukemia (NCT02782546, NCT03081780, NCT01787474), Acute Myeloid Leukemia in Children (NCT03068819), Plasma Cell Leukemia (NCT01729091), Acute Erythroid Leukemia, Acute Megakaryoblastic Leukemia, Chronic Myelomonocytic Leukemia, Myelodysplastic Syndrome (NCT01823198). The cells produced by the methods described herein can be used to treat or alleviate solid cancers, such as Neuroblastoma, Ewing Sarcoma, Rhabdomyosarcoma (NCT01857934, NCT02100891), Pancreatic cancer, Colon/rectal cancer, Non-Small-Cell Lung cancer Gastric Cancer, Head and Neck cancer, Squamous Cell Carcinoma, Breast Cancer, Hepatocellular Carcinoma, Renal Cell Carcinoma, Melanoma, Urothelial Carcinoma, Cervical Cancer, Merkel Cell Carcinoma (NCT00720785, NCT03319459, NCT03841110), Ovarian Carcinoma, Fallopian Tube Carcinoma, Primary Peritoneal Carcinoma (NCT03539406), Medulloblastoma, Ependymoma (NCT02271711), Malignant Bone Neoplasm, Lip and Oral Cavity Carcinoma, Endocrine Neoplasm, Male Reproductive System Neoplasm, Mesothelioma, Oral Neoplasm, Pharyngeal Neoplasm, Bone Neoplasm, Thyroid Gland Neoplasm (NCT03420963).

Sequence Overview

TABLE 1
Sequence overview.

| SEQ. ID. NO. | Sequence description | Sequence type |
|---|---|---|
| SEQ. ID. NO. 1 | ETS1 | Protein |
| SEQ. ID. NO. 2 | TBET | Protein |
| SEQ. ID. NO. 3 | NFIL3 | Protein |
| SEQ. ID. NO. 4 | EOMES variant 1 | Protein |
| SEQ. ID. NO. 5 | EOMES variant 2 | Protein |
| SEQ. ID. NO. 6 | EOMES variant 3 | Protein |
| SEQ. ID. NO. 7 | ID2 | Protein |
| SEQ. ID. NO. 8 | GATA3 | Protein |
| SEQ. ID. NO. 9 | ZFP105 | Protein |
| SEQ. ID. NO. 10 | IKZF3 | Protein |
| SEQ. ID. NO. 11 | TOX | Protein |
| SEQ. ID. NO. 12 | RUNX3 | Protein |
| SEQ. ID. NO. 13 | KLF12 | Protein |
| SEQ. ID. NO. 14 | ZEB2 | Protein |
| SEQ. ID. NO. 15 | IRF2 | Protein |
| SEQ. ID. NO. 16 | STAT5 | Protein |
| SEQ. ID. NO. 17 | IKZF1 | Protein |
| SEQ. ID. NO. 18 | ELF4 | Protein |
| SEQ. ID. NO. 19 | ZBTB16 | Protein |
| SEQ. ID. NO. 20 | GATA2 | Protein |
| SEQ. ID. NO. 21 | ELF1 | Protein |
| SEQ. ID. NO. 22 | ETS1 | DNA |
| SEQ. ID. NO. 23 | TBET | DNA |
| SEQ. ID. NO. 24 | NFIL3 | DNA |
| SEQ. ID. NO. 25 | EOMES variant 1 | DNA |
| SEQ. ID. NO. 26 | EOMES variant 2 | DNA |
| SEQ. ID. NO. 27 | EOMES variant 3 | DNA |
| SEQ. ID. NO. 28 | ID2 | DNA |
| SEQ. ID. NO. 29 | GATA3 | DNA |
| SEQ. ID. NO. 30 | ZFP105 | DNA |
| SEQ. ID. NO. 31 | IKZF3 | DNA |
| SEQ. ID. NO. 32 | TOX | DNA |
| SEQ. ID. NO. 33 | RUNX3 | DNA |
| SEQ. ID. NO. 34 | KLF12 | DNA |
| SEQ. ID. NO. 35 | ZEB2 | DNA |
| SEQ. ID. NO. 36 | IRF2 | DNA |
| SEQ. ID. NO. 37 | STAT5 | DNA |
| SEQ. ID. NO. 38 | IKZF1 | DNA |
| SEQ. ID. NO. 39 | ELF4 | DNA |
| SEQ. ID. NO. 40 | ZBTB16 | DNA |
| SEQ. ID. NO. 41 | GATA2 | DNA |
| SEQ. ID. NO. 42 | ELF1 | DNA |

### REFERENCES

Bachanova, V. et al. Clearance of acute myeloid leukemia by haploidentical natural killer cells is improved using IL-2 diphtheria toxin fusion protein. Blood 123, 3855-3863 (2014).
Barry, K. C. et al. A natural killer-dendritic cell axis defines checkpoint therapy-responsive tumor microenvironments. Nat. Med. 24, 1178-1191 (2018).
Bottcher, J. P. et al. NK cells stimulate recruitment of cDC1 into the tumor microenvironment promoting cancer immune control. Cell 172, 1022-1037 (2018).
Boudreau J E, et al., Cell-extrinsic MHC class I molecule engagement augments human NK cell education programmed by cell-intrinsic MHC class I. Immunity 45, 280-291 (2016).
Bezman N A, et al., Molecular definition of the identity and activation of natural killer cells. Nature Immunology 13, 1000-1009 (2012).
E. Vivier et al., Innate or Adaptive Immunity? The Example of Natural Killer Cells. Science 331, 44-49 (2011).
Eckelhart, E., et al., A novel Ncr1-Cre mouse reveals the essential role of STAT5 for NK-cell survival and development. Blood 117, 1565-1573 (2011).
Firas, J., Polo, J., Gough, J., Hayashizaki, Y., Rackham, O., Cell Reprogramming, Patent WO2019177936A1, 2017
Gomes A M, et al. Cooperative Transcription Factor Induction Mediates Hemogenic Reprogramming. Cell Rep 25, 2821-2835.e7 (2018)
Graf T., Enver T., Forcing Cells to Change Lineages, Nature 462, 587-94 (2009)
Lopez-Soto, A., et al., Control of metastasis by NK cells. Cancer Cell 32, 135-154 (2017).
Noriko Shimasaki et al. NK cells for cancer immunotherapy. Nat Rev Drug Discov 19, 200-218 (2020).
Passweg, J. R. et al. Purified donor NK-lymphocyte infusion to consolidate engraftment after haploidentical stem cell transplantation. Leukemia 18, 1835-1838 (2004).
Pereira C F, et al., Reprogramming cell fates: Insights from combinatorial approaches. Ann N Y Acad Sci 1266 7-17 (2012).
Pereira C F, et al., Induction of a hemogenic program in mouse fibroblasts. Cell Stem Cell 13,205-18 (2013).
Renoux, V. M. et al., Identification of a human natural killer cell lineage-restricted progenitor in fetal and adult tissues. Immunity 43, 394-407 (2015).
Rosa F F, et al., Direct reprogramming of fibroblasts into antigen-presenting dendritic cells. Sci Immunol 3(30):4292 (2018)
Takahashi K, Yamanaka S. Induction of Pluripotent Stem Cells from Mouse Embryonic and Adult Fibroblast Cultures by Defined Factors. Cell 126(4):663-76 (2006).
Tam, Y. K., et al., Ex vivo expansion of the highly cytotoxic human natural killer-92 cell-line under current good manufacturing practice conditions for clinical adoptive cellular immunotherapy. Cytotherapy 5, 259-272 (2003).
Ye Li et al., Human iPSC-Derived Natural Killer Cells Engineered with Chimeric Antigen Receptors Enhance Antitumor Activity. Cell Stem Cell 23, 181-192 (2018).

1. At least one polynucleotide encoding a combination of at least three different transcription factors selected from the group consisting of: ETS1, TBET, NFIL3 and EOMES.

2. The at least one polynucleotide according to claim 1, wherein the combination of at least three transcription factors is a combination of the four transcription factors ETS1, TBET, NFIL3 and EOMES.

3. The at least one polynucleotide according to any one of the preceding claims, wherein the combination of at least three transcription factors comprises an amino acid sequence selected from the group consisting of: SEQ. ID. NO: 1 to SEQ. ID. NO: 6.

4. The at least one polynucleotide according to any one of the preceding claims, wherein the combination of at least three transcription factors are encoded by a polynucleotide sequence selected from the group consisting of: SEQ. ID. NO: 22 to SEQ. ID. NO: 27, or a sequence with at least 80%, such as at least 85%, such as at least 90%, such as at least 95% sequence identity thereof.

5. The at least one polynucleotide according to any one of the preceding claims, wherein the transcription factor ETS1 comprises the amino acid sequence SEQ. ID. NO: 1 or a biologically active variant thereof.

6. The at least one polynucleotide according to claim 5, wherein the polynucleotide encoding SEQ. ID. NO: 1 is SEQ. ID. NO: 22, or a sequence with at least 80%, such as at least 85%, such as at least 90%, such as at least 95% sequence identity thereof.

7. The at least one polynucleotide according to any one of the preceding claims, wherein the transcription factor TBET comprises the amino acid sequence SEQ. ID. NO: 2, or a biologically active variant thereof.

8. The at least one polynucleotide according to claim 7, wherein the polynucleotide encoding SEQ. ID. NO: 2 is SEQ. ID. NO: 23, or a sequence with at least 80%, such as at least 85%, such as at least 90%, such as at least 95% sequence identity thereof.

9. The at least one polynucleotide according to any one of the preceding claims, wherein the transcription factor NFIL3 comprises the amino acid sequence SEQ. ID. NO: 3, or a biologically active variant thereof.

10. The at least one polynucleotide according to claim 9, wherein the polynucleotide encoding SEQ. ID. NO: 3 is SEQ. ID. NO: 24, or a sequence with at least 80%, such as at least 85%, such as at least 90%, such as at least 95% sequence identity thereof.

11. The at least one polynucleotide according to any one of the preceding claims, wherein the transcription factor EOMES comprises the amino acid sequence SEQ. ID. NO: 4, or a biologically active variant thereof.

12. The at least one polynucleotide according to claim 11, wherein the biologically active variant comprises a sequence selected from the group consisting of: SEQ. ID. NOs: 5 to 6.

13. The at least one polynucleotide according to any one of claims 11 to 12, wherein the polynucleotide encoding SEQ. ID. NOs: 4 to 6 is selected from the group consisting of: SEQ. ID. NO: 25, SEQ. ID. NO: 26 and SEQ. ID. NO: 27, or a sequence with at least 80%, such as at least 85%, such as at least 90%, such as at least 95% sequence identity thereof.

14. The at least one polynucleotide according to any one of the preceding claims, wherein the combination of at least three transcription factors is selected from the group consisting of:
ETS1, TBET, NFIL3 and EOMES;
ETS1, TBET and NFIL3;
ETS1, TBET and EOMES;
ETS1, NFIL3 and EOMES; and
TBET, NFIL3 and EOMES.

15. The at least one polynucleotide according to any one of the preceding claims, wherein the combination further comprises one or more transcription factors selected from the group consisting of: ID2, GATA3, ZFP105, IKZF3, TOX, RUNX3, KLF12, ZEB2, IRF2, STAT5, IKZF1, ELF4, ZBTB16, GATA2 and ELF1.

16. A construct or vector comprising the at least one polynucleotide according to any one of claims 1 to 15.

17. The construct or vector according to claim 16, wherein the vector is a viral vector.

18. The construct or vector according to claim 17, wherein the vector is selected from the group consisting of: lentiviral vector, retroviral vector, adenoviral vector, herpes viral vector, pox viral vector, flaviviral vector, rabdoviral vector, paramyxoviral vector, adeno-associated viral vector, reoviral vector, papillomaviral vector, picornaviral vector, caliciviral vector, hepadnaviral vector, togaviral vector, coronaviral vector, hepatitis viral vector, orthomyxoviral vector, bunyaviral vector and filoviral vector.

19. The construct or vector according to claim 17, wherein the viral vector is a member of the family of lentiviruses.

20. The construct or vector according to any one of claims 16 to 19, wherein the vector is an oncolytic vector.

21. The construct or vector according to any one of claims 16 to 20, wherein the construct or vector is synthetic mRNA, naked alphavirus RNA replicon or naked flavivirus RNA replicon.

22. The construct or vector according to any one of claims 16 to 20, wherein the vector further comprises a post-translational regulatory element (PRE) sequence.

23. The construct or vector according to claim 22, wherein the PRE sequence is selected from the group consisting of: a Woodchuck PRE (WPRE) or a hepatitis B virus (HBV) PRE (HPRE).

24. The construct or vector according to any one of claims 16 to 23, wherein the vector further comprises a promoter sequence controlling the transcription of the at least one polynucleotide according to any one of claims 1 to 15.

25. The construct or vector according to claim 24, wherein the promoter sequence is selected from the group consisting of: Spleen Focus-Forming Virus (SFFV) promoter, cytomegalovirus (CMV) promoter, phosphoglycerate kinase (PGK) promoter, myelin basic protein (MBP) promoter, glial fibrillary acidic protein (GFAP) promoter, modified MoMuLV LTR containing myeloproliferative sarcoma virus enhancer (MNDU3), ubiquitin C promoter, EF-1 alpha promoter, or murine stem cell virus (MSCV) promoter.

26. The construct or vector according to claim 16, wherein the construct or vector is a plasmid.

27. The construct of vector according to any one of claims 19 to 26, wherein the at least three transcription factors of the at least one polynucleotide is, in the following sequential order from 5′ to 3′, selected from the group consisting of:
ETS1, TBET, NFIL3 and EOMES;
TBET, ETS1, NFIL3 and EOMES;
NFIL3, ETS1, TBET and EOMES;
ETS1, NFIL3, TBET and EOMES;
TBET, NFIL3, ETS1 and EOMES;
NFIL3, TBET, ETS1 and EOMES;
NFIL3, TBET, EOMES and ETS1;
TBET, NFIL3, EOMES and ETS1;
EOMES, NFIL3, TBET and ETS1;
NFIL3, EOMES, TBET and ETS1;
TBET, EOMES, NFIL3 and ETS1;
EOMES, TBET, NFIL3 and ETS1;
EOMES, ETS1, NFIL3 and TBET;
ETS1, EOMES, NFIL3 and TBET;
NFIL3, EOMES, ETS1 and TBET;
EOMES, NFIL3, ETS1 and TBET;
ETS1, NFIL3, EOMES and TBET;
NFIL3, ETS1, EOMES and TBET;
TBET, ETS1, EOMES and NFIL3;
ETS1, TBET, EOMES and NFIL3;
EOMES, TBET, ETS1 and NFIL3;
TBET, EOMES, ETS1 and NFIL3;
ETS1, EOMES, TBET and NFIL3;
EOMES, ETS1, TBET and NFIL3;
ETS1, TBET and NFIL3;
TBET, ETS1 and NFIL3;
NFIL3, ETS1 and TBET;
ETS1, NFIL3 and TBET;
TBET, NFIL3 and ETS1;
NFIL3, TBET and ETS1;
ETS1, TBET and EOMES;
TBET, ETS1 and EOMES;
EOMES, ETS1 and TBET;
ETS1, EOMES and TBET;
TBET, EOMES and ETS1;
EOMES, TBET and ETS1;
ETS1, NFIL3 and EOMES;
NFIL3, ETS1 and EOMES;
EOMES, ETS1 and NFIL3;
ETS1, EOMES and NFIL3;
NFIL3, EOMES and ETS1;
EOMES, NFIL3 and ETS1;
TBET, NFIL3 and EOMES;
NFIL3, TBET and EOMES;
EOMES, TBET and NFIL3;
TBET, EOMES and NFIL3;
NFIL3, EOMES and TBET;
and EOMES, NFIL3 and TBET.

28. The construct or vector according to any one of claims 16 to 27, wherein the construct or vector is a CRISPR/CAS activation system.

29. A cell comprising the at least one polynucleotide according to any one of claims 1 to 15, and/or the construct or vector according to any one of claims 16 to 28.

30. The cell according to claim 29, wherein the cell is a mammalian cell.

31. The cell according to any one of claims 29 to 30, wherein the cell is a human cell.

32. The cell according to any one of claims 29 to 31, wherein the cell is selected from the group consisting of: a stem cell, a differentiated cell, a cancer cell or a tumor cell.

33. The cell according to claim 32, wherein the stem cell is selected from the group consisting of: a pluripotent stem cell and a multipotent stem cell, such as a mesenchymal stem cell, and hematopoietic stem cell.

34. The cell according to claim 32, wherein the differentiated cell is any somatic cell.

35. The cell according to claim 34, wherein the cell is selected from the group consisting of: a fibroblast or a hematopoietic cell such as a monocyte or a mast cell.

36. The cell according to any one of claims 29 to 35, wherein the cell is NCR1 positive.

37. The cell according to any one of claims 29 to 36 wherein the cell is CD34 positive.

38. The cell according to any one of claims 29 to 37 wherein the cell is CD56 positive.

39. A pharmaceutical composition comprising the at least one polynucleotide according to any one of claims 1 to 15, the construct or vector according to any one of claims 16 to 28, and/or the cell according to any one of claims 29 to 38.

40. The pharmaceutical composition according to claim 39, wherein the composition further comprises a suitable carrier.

41. A method for reprogramming or inducing a cell into a natural killer cell or progenitor, the method comprising the following steps:
a. transducing a cell with the at least one polynucleotide according to any one of claims 1 to 15, or the construct or vector according to any one of claims 16 to 28, or the pharmaceutical composition according to any one of claims 39 to 40;
b. culturing and expanding the transduced cell in a cell media; and
c. obtaining a reprogrammed cell.

42. The method according to claim 41, wherein the method further comprises culturing the transduced cell in a media comprising one or more cytokine(s).

43. The method according to claim 42 wherein the one or more cytokine(s) are selected from the group consisting of: SCF, FLT3L, IL-3, IL-7 and IL-15

44. The method according to any one of claims 41 to 43, wherein the cell is a mammalian cell.

45. The method according to any one of claims 41 to 44, wherein the cell is a human cell.

46. The method according to any one of claims 41 to 45, wherein the cell is selected from the group consisting of: a stem cell, a differentiated cell and a cancer cell.

47. The method according to claim 46, wherein the stem cell is selected from the group consisting of: a mesenchymal stem cell, a pluripotent stem cell and hematopoietic stem cell.

48. The method according to claim 46, wherein the differentiated cell is selected from the group consisting of: a fibroblast and a monocyte.

49. The method according to any one of claims 41 to 48, wherein the transduced cell is cultured during at least 2 days, such as at least 5 days, such as at least 8 days, such as at least 10 days, such as at least 12 days.

50. The method according to any one of claims 41 to 49, wherein the reprogrammed cell is NCR1 positive.

51. The method according to any one of claims 41 to 50, wherein the reprogrammed cell is CD34 positive.

52. The method according to any one of claims 41 to 51, wherein the reprogrammed cell is CD56 positive.

53. An induced natural killer cell obtained by the method according to any one of claims 41 to 52.

54. The induced natural killer cell according to claim 53, wherein the induced cell is NCR1 positive.

55. The induced natural killer cell according to any one of claims 53 to 54, wherein the induced cell is CD34 positive.

56. The induced natural killer cell according to any one of claims 53 to 55, wherein the induced cell is CD56 positive.

57. The at least one polynucleotide according to any one of claims 1 to 15, the construct or vector according to any one of claims 16 to 28, the cell according to any one of claims 29 to 38, the pharmaceutical composition according to any one of claims 39 to 40, and/or the induced natural killer cell according to any one of claims 53 to 56, for use in medicine.

58. The at least one polynucleotide according to any one of claims 1 to 15, the construct or vector according to any one of claims 16 to 28, the cell according to any one of claims 29 to 38, the pharmaceutical composition according to any one of claims 39 to 40, and/or the induced natural killer cell according to any one of claims 53 to 56, for use in the treatment of cancer.

59. The at least one polynucleotide according to any one of claims 1 to 15, the construct or vector according to any one of claims 16 to 28, the cell according to any one of claims 29 to 38, the composition according to any one of claims 39 to 40, and/or the induced natural killer cell according to any one of claims 53 to 56, for use in immunotherapy.
